(19) United States
(12) Patent Application Publication
(10) Pub. No.: US 2023/0190714 A1
(43) Pub. Date: Jun. 22, 2023

(54) PRIMARY METHODS AND PROCESSES FOR LIFE EXTENSION IN MODERN-DAY HUMANS

(71) Applicant: Richard Postrel, Miami Beach, FL (US)

(72) Inventor: Richard Postrel, Miami Beach, FL (US)

(21) Appl. No.: 16/602,663

(22) PCT Filed: Mar. 18, 2018
(86) PCT No.: PCT/US18/23046

§ 371 Date: Nov. 15, 2019

Related U.S. Application Data
(60) Provisional application No. 62/504,850, filed on May 11, 2017.

(51) Int. Cl. A61K 31/436; A61K 31/198; A61K 31/353; A61K 45/06

(52) U.S. Cl. CPC A61K 31/436; A61K 31/198; A61K 31/353; A61K 45/06

(57) ABSTRACT

This invention explains how to improve and/or extend human life by optimizing metabolic processes. This patent teaches how to reestablish or correct pathways that have been altered either by biochemical stress or by genetic mutation. The body's energetic mitochondrial machinery is programmed for optimization at birth. As events are encountered throughout its lifecycle the cells respond to these stresses by altering their metabolic configurations to meet the immediate demands. Each of these successive adaptive biochemical reactions cumulatively magnifies previous compensatory switches from the original optimal metabolic pathways and diminishes the individual's quality of life and lifespan. As we age these opportunistic adjustments continue to compound and further reduce metabolic efficiency to levels that significantly compromise health and longevity. Modern technology, including molecular biology and micro or nano electronics, is applied to assess the multiple impaired metabolic pathways in an individual and to employ biologic interventions and tools that eliminate those diversions and/or correct genetic and/or epigenetic mutations.

### BACKGROUND

This invention explains how to improve and/or extend human life by optimizing metabolic processes. Life on earth has adapted over the eons from metabolism in an oxygen free environment to lifeforms highly dependent on the oxygen produced by plants, from essentially aquatic to successful life on land, etc. One requirement for survival is that life must respond to its changing environments, temperature, food availability, disease, etc. As individuals encounter different externalities their metabolisms are programmed to adapt metabolic pathways responsive to these changes. The adapted state thereby becomes the new “normal”, the start point for responses to subsequent externalities. With each adaptation, the individual diverges from the preprogrammed ideal state operative when they entered the changing earth. The adapted metabolism is a characteristic of aging. This patent teaches how to reestablish or correct pathways that have been altered either by biochemical stress or by genetic mutation. The body's energetic mitochondrial machinery is programmed for optimization at birth. As events are encountered throughout its lifecycle the cells respond to these stresses by altering their metabolic configurations to meet the immediate demands. Each of these successive adaptive biochemical reactions cumulatively magnifies previous compensatory switches from the original optimal metabolic pathways and diminishes the individual's quality of life and lifespan. As we age these opportunistic adjustments continue to compound and further reduce metabolic efficiency to levels that significantly compromise health and longevity. Modern technology, including molecular biology and micro or nano electronics, is applied to assess the multiple impaired metabolic pathways in an individual and to employ biologic interventions and tools that eliminate those diversions and/or correct genetic and/or epigenetic mutations.

We now know that life on the planet earth existed 4.1 to 4.3 billion years ago in what is now Australia. As the planet has matured life forms have adapted and become more diverse and complex. Evidence of primitive cells, dates back almost 4 billion years with archaea and bacteria diverging a half billion years later. Life on earth has an extended history. Though none of these primitive organisms are known to exist today, we humans and all life forms on earth survive as their progeny.

The earth has experienced massive change. The conditions present at the dawn of life would be toxic to most of today's life forms—oxygen was not present in the atmosphere until life on earth had existed for over a billion years. Similarly, the conditions present today would not support the early life forms—today's temperatures and the presence of a strong oxidizing agent (oxygen) would make their survivals impossible. History is defined by constant change, cause and effect, stress and strain, action and reaction. Life must adapt.

In the grand cycle of life each cell or organism must adapt or be replaced by “progeny” better aligned with its current immediate environment. A part of the adaptation process invokes a sort of experimentation process as the progeny experience a changed set of conditions. As larger organisms came into existence, the cells making up those organisms adapted and differentiated to the complex beings we have today. Humans on earth are relatively young having been present only 5-10 million years, but even in this short time the requirements for humans to survive have immensely changed. Humans learned control fire removing many restrictions on human life; they could expand to colder climates, they could eat a larger variety of foods, they could spend less life obtaining, processing and chewing food, they could remain active after sundown.

The earth even in its recent and minimal (less than 0.1% of the time life has been here) humanocene (period of humans walking the earth)¹, has seen significant changes. In the most recent 5 million years, the first two million years were relatively ice free. About three million years ago Greenland and the Artic became glaciated as average earth temperatures plummeted. About 2.5 million years ago ice ages cycled between glacial and interglacial periods on about 40,000 year cycles before switching to about 100,000 year cycles about one million years ago. It wasn't until the most recent glacial retreat, about 12,000 years ago that large brained humans formed farming cultures. For each glacial and interglacial period many generations of our ancestral hominids adapted their activities and movements to optimally manage species survival. ¹ Humanocene—period of human/hominid development in the developing earth (4-5 million years) as contrasted with Anthropocene—the period where humans significantly modify their environment.

For a species to survive, individuals of that species must continuously adapt to their changed environments so they can survive to reproduce more individuals of the species. For individuals of a species to survive, cells of the individuals must grow and adapt to provide sufficient structure and support to maintain the organism. Simply packing and moving to a location similar to that being vacated—possibly during a change in seasons, change in glacial cycle or change in temperatures or food supplies requires significant physical effort to minimize stress on the individual and minimize required intracellular or genomic changes. But with each change the body adapts. Frequent moves will build and maintain these required muscles. Muscle cells will adapt to provide creatine kinase and supportive mitochondrial oxidative metabolism. Daughter cells will maintain and continue the adaptations to support the whole organism's adaptations. But even in this “recent” period organisms faced many changes in climate stress, including, but not limited to: ultra violet stress, temperature stress, periods of drought/dehydration food stress, chemical stress transient and more permanent adaptations were formed. Adaptations made in response to one set of conditions force further adaptations to compensate for the legacy adaptations. One obvious example is the bipedal adaptations that is maintained despite some conditions that might favor more stability. But overall the bipedal characteristic resulted in survival advantage. And the organism then locomoted to environments where the bipedal characteristic was more favored. Similar considerations apply to individual cells and even sub-components of cells. Once one adaptation is made, future adaptations are driven to support survival of the adapted status.

Humans learned to plant and harvest their own food and to breed animals for work and food. Humans could expend their life's capital on other pursuits, such as science, understanding their surroundings. We humans specialized various organs and cells in the organs for different tasks. For example, our liver, processes most foods taken into our bodies, our circulatory system delivers needed chemical components throughout our entire bodies, our brain coordinates activities of the various organs and through memory has allowed us to learn. Our kidneys adapt to rid the body of various wastes. The liver adapts to process deliverables from the intestines and circulation. And with our brains, we have increased our understanding of our planet, life on our planet and ourselves, including the various cells and their specializations necessary to support life as we know it.

During this specialization of various cell types and their contributions to the survival of the greater organism the cells of the organism must change as the organism grows and matures. Sometimes after a cell has completed its specialized tasks, removal of that cell makes the organism better off. Cell death is built into our growth and development. For example, cells of our baby teeth experience death as adult teeth develop for the grown-up jaw.

The cells of our bodies have been adapting over time to optimize survival for the conditions the organism experiences and provides for its cells internally. But these adaptations are responsive to change and therefore lag in time behind the changes encountered, the cells we have today may have been optimal for a previous time, but our times are constantly in flux. One advantage we have now, in part due to our specialized brain, is an improved understanding of our human bodies: including our genetic material, many proteins that coordinate chemical reactions within cells and ways to steer change (or in some cases entirely change) certain functions through manipulating various parts and/or components of cells to up-regulate, down-regulate, restart, refocus or eliminate one or more or our biochemical reactions.

We now can understand that our bodies' and cells of our bodies' changing life needs may, under our current conditions on earth, unnecessarily shorten most human lifespans. The increasing understanding of our bodies and outside physical, chemical and biological effects on our health and lifespans has allowed us to increase life expectancy from about 32 years for a person born in 1900 to over 70 years for a person born today. But increased longevity has come at a cost where many individuals spend perhaps their last decade in a state of declined physical and mental abilities. We can now use our developed and developing knowledge to intervene early in the processes leading to decline and maintain healthful life for an extended lifespan.

Humans are vertebrate animals of class Mammalia. This class has developed multiple features that are not present in other animal or plant classes. Other classes though have developed their own distinct survival mechanisms and therefore can be considered on a complexity par with humans. Selection over life's eons has resulted in interaction of thousands of features and variations of features in the diverse organisms, but many features are common to all. For example, all live organisms, and even viruses, use nucleic acid to instruct life's processes, including reproduction; most organisms have a membrane to encase their life form from its environment; catalysts (proteins) interact with cell components and environment to sustain continuity over time.

Darwinian Theory spoke to survival of the fittest. Whatever makes best use of its tools and its environment to achieve immortality or to produce continuous generations of offspring will continue to be with us through years, decades, centuries, epochs and eons. According to this theory, eons of selective pressures have produced the complexities of biology with us today. But even the smallest packages of life that we have with us today have faced the same eons of selective pressure as humans. Organisms may have adapted over the eons, but each cell in the organism must therefore be a product of the same pressures and adaptations. Whether life on earth is strictly the result of selective pressures, a result of some intelligent engineering, an experiment conducted by aliens, or something else, is immaterial—we live in today's complex world and now have developed understanding and tools to use as we choose for our benefits. Human longevity increased with farming, with pottery, with sanitary sewers, with improved understanding of germ theory, with enhanced medical intervention, etc. In the medical field we have progressed from shunning “different” or diseased persons, to accepting the pathologies of disease including the understanding that even microorganisms do not spontaneously generate, and that genetic code underlies biologic events.

We benefitted from observation of clear skinned milkmaids through to a constantly growing understanding of virus and bacteria. Rather than just observing death and occasionally disease leading to death, we learned that body temperature and urine sweetness were markers useful for managing or treating diseases. We now have available the complete human genome of thousands of individuals. We have x-ray, PET, MRI ultrasound and other imaging tools for non-invasive observations within our bodies. A doctor can choose from thousands of laboratory tests to use for diagnosis. Rather than merely observing present physical status, we can use various tests and tools to assess past events within our bodies, and now, to accurately predict: days, weeks and sometimes years in advance of our future health and various interventions that might improve it. We now can apply the knowledge and tools available to us, to once again substantially improve human life quality and longevity.

As we can see further into the future, essentially by recognizing disease events at stages before the chemical changes are casually observable outside our bodies, we can intervene earlier in disease processes, with less intense therapy, but with earlier, better and less expensive results. The key is to recognize small imbalances early before they provoke major tipping points in our cell's responsive metabolic adaptations. Small changes in ion fluxes or gradients, electrochemical potentials, membrane changes that might include receptor up-regulation or down-regulation, if addressed at an early stage can prevent the cell from digressing further down its declining or diseased path and maintain better health for an extended time period.

Homo sapiens is the taxonomic identifier for the modern human. Humans are members of the eukaryotic domain and thus comprise cells having organelles including, but not limited to: nucleus, endoplasmic reticulum, golgi, lysosomes, peroxisomes, vesicles, cytoskeleton, mitochondria, etc.

The cells in one individual human are not identical. The diverse tissues have cells specialized to perform the functions of that tissue. The multiple functions within a tissue require cells, even cells within the same tissue, to specialize. As an example, the lung requires cells to deliver oxygen depleted blood and to remove carbon dioxide depleted blood; specialized cells provide mechanical structure; cells make and secrete fluids, signaling substances and nutrients for neighbor cells; and immune cells counter disease. The human organism, like other large animals contains multiple micro environments where a diverse agglomeration of differentially developed cells cooperates to sustain the human organism and species. Differentiation of cells to serve a special purpose is one type of adaptation.

### SUMMARY OF THE INVENTION

Human life involves multiple trillions of cell divisions during a normal lifetime. And each cell division involves thousands of coordinated events, including maintenance of organelles that may individually require several thousands of biochemical creations within and without the organelle. It's amazing to consider that all these multi-trillions of steps can coordinate sufficiently to bring us to adulthood and beyond. However, as every adult knows and feels, not all the reactions within our bodies are as optimal as they once were. The biochemical events in our bodies today are not the more vigorous life enhancing events we experienced in our past. During our lifetimes our cells have accumulated baggage—changes from the more optimal balance the cells originally had before stresses forced changes in their structures and metabolisms.

When metabolism is functioning optimally, the cell delivers appropriate substrate to metabolically relevant sub-cellular structures; the substrate is processed; and the products are delivered to the next step in that pathway. When an atypical result occurs, it may be impossible for the next reaction to occur. This by-product may be secreted from the cell, may be used in a different pathway, may bind or otherwise interfere with another molecule in the cell, may be degraded by scavenging actions within the cell, or may just float around getting in the way reversibly binding and impacting assigned ability and availability of pseudo-random biomolecules.

The cell has multiple means for correcting or discarding metabolic errors. But often when an unexpected substrate (perhaps a drug or toxin or just an unaccustomed food not encountered during maturation of our genome) or an abnormal amount of substrate presents, the cell will switch its biochemical machinery in response to the stress, perhaps activating a kinase, inducing transcription of an enzyme or receptor, tagging an enzyme for recycling, or epigenetically altering the activity of genetic material. Sometimes these changes are not easily reversible but managed in the cell. Sometimes these may lead to cell death through initiation of apoptosis. Generally, the adaptations trigger changes in related pathways which may produce a small or large imbalance of the cell's original metabolic status.

During cell division, another type of error can occur. Cell division requires duplication of the cell's instruction set that is written in the nuclear DNA (nDNA). DNA duplication of a genome requires millions of individual chemical reactions. Any of these may go wrong. The cell has specific repair pathways to correct these rare DNA errors. But on rare occasion the correction mechanics can malfunction. The BRCA breast cancer gene mutations are examples where the repair processes are compromised. Defects in any of our protein managing processes e.g., ubiquitination/deubiquitination may compromise DNA protection/repair and result in faulty genomic instructions. Nuclear DNA is protected by nuclear histone proteins. Mitochondria simply do not have nuclear histones and thus have increased DNA (mtDNA) fragility. Although mtDNA may be protected from some damaging molecules by TFAM, mtDNA appears to mutate at a rate in excess of an order of magnitude than detected in nDNA. Several nDNA encoded proteins that repair nDNA have been observed in mitochondrion and are assumed to carry out similar functions there. Damage causing modified nucleotides can result in polymerase arrest preventing copying of the damaged DNA molecule. Endonuclease G is active particularly in degrading entire oxidized mtDNA which is often present in mitochondria with mutated mtDNA thereby degrading a damaged mitochondrion's ability to function which often initiates mitophagy.

In cells with germline or somatic mutation, when repair is not as vigorous as found in a normal (wild-type) cell, multiple genetic mistakes may compound within a cell.

Our cellular engine room, the mitochondrion, where chemical energy from sugar is converted to ubiquitously useful triphosphates, has only a 16.5-kb genome. But each mitochondrion may have at least a dozen or a score of separate genomic molecules. Each of which may develop its own somatic mutations. When a significant proportion of the mtDNA molecules in a mitochondrion have deleteriously mutated the mitochondrion or the cell may initiate a mitophagic process, thus correcting for mutation events by eliminating the mutant product. There appears to be processes, only recently beginning to be understood where individual genomes in a mitochondrion are repaired or dismantled. Often damaged mtDNA do not replicate as efficiently as wilder type mtDNA and thus decrease in numbers following fusion and fission events.

Mutations in mtDNA are linked to a spectrum of other pathologies including cancer, diabetes, cardiovascular diseases, and neurodegenerative disorders, as well as the normal process of aging. Identified mutations in germline mtDNA are associated with over 200 [mitochondrial] diseases that may manifest as “common” diseases such as diabetes, cancer, male infertility, Parkinson's, Alzheimer's diseases, etc. Mitochondrial abnormalities have been documented in all major neurodegenerative disorders including Alzheimer's disease, Parkinson's disease, Huntington's disease and Lou Gehrig's disease. Mitochondrial DNA damage and dysfunction may participate in the primary disease processes or be downstream responding to, for example, accumulation of pathogenic aldehydes and/or proteins. For example, Aβ protein, a protein deposited in the brains of Alzheimer's disease patients, can be found in mitochondria and is associated with reduced activity of complexes 4 and 5 and reduced O₂ metabolism, but increased H₂O₂; Parkinson's disease cells show decreased complex 1 activity and increased reactive oxygen species (ROS) production; diseased Cu,Zn-superoxide dismutase (SOD1) accumulates in the outer mitochondrial matrix (OMM), affect Ca⁺⁺ levels and electron transport chain activity.

In mammals, mtDNA is maternally inherited, the sperm fail to contribute mitochondria during the fertilization process. But since germline cells that produce the oocytes undergo few divisions in comparison to somatic cells mtDNA therefore will have greater stability thereby allowing mtDNA (one-million to ten-million copies in typical oocytes) to serve as a maternal lineage marker. Still heteroplasmy, the presence of detectable amounts of differentiated DNA, increases with the age of the mother.

Heteroplasmy is variable generation to generation, i.e., the ratio of heteroplasmic species can vary immensely from mother to daughter. One hypothesis for this variance is the “bottleneck” effect where the selection of the mitochondria during cell division may group one population of mtDNA sequences more in one of the daughter cells than the other. This suggests that individual mitochondria are more homoplasmic than heteroplasmic. If this is in fact true, the mitochondrion selects and eliminates or favors particular mtDNAs over others. This selection is either biased towards survival of the cell or cells selecting poorly simply fail to survive and produce daughter cells as fertilely as those with more adaptive mtDNA. Thus, under a given set of conditions cell survival, dependent on mitochondrial survival, will express selected adaptations compatible with previous adaptations (which may no longer be usefully relevant) and current conditions. Restoring mitochondria and the hosting cells to preadaptive conditions favorable to current conditions is therefore a beneficial outcome.

The breast cancer BRCA gene mutations are not extremely rare and tend to be found in specific originating populations. For example, one BRCA mutation slightly increases the female:male ratio of offspring. This one mutation could cause an increase in the percentage of women with this mutation so long as the BRCA women reached reproductive fulfillment. Similarly, germline mtDNA mutations that are associated with the many diseases are not one-off events. At some time under some set of conditions these mutations rendered a survival benefit. A similar concept is operational in the cells of our bodies. Adaptations in response to a set of stresses reset the cell and its organelles to these stresses. But when these stresses are removed, the adaptations themselves provide a stress compared to the eons of selection to optimize health and survival.

So, the cell in its drive to survive can reprocess, reconfigure, recycle or otherwise correct or eliminate many mistakes of metabolism. The multiple pathways within the cell interweave and cooperate to upregulate and down regulate activities in multidimensional feedback loops to maintain the cell's metabolism and in normal operations to match the metabolism to the needs of the organism. Within the cell, each part must function appropriately within its pathway to support both the cell's and the organism's needs. The cell has a process called “autophagy” that can reprocess and eliminate unneeded or poorly functioning organelles, e.g., mitochondria, whose autophagic process has its own name, mitophagy. The organism has immune systems that can recognize improper cells. The organism has at its disposal genes that instruct a misbehaving cell: i) to take corrective measures and ii) to cease all functions when corrective measures are inadequate.

But we know that nothing is perfect. Sometimes the cell may fail to eliminate mutated mitochondria or may miss correcting a nDNA error. These errors would then be promulgated with every new cell division. But since the cell is a part of the organism and the cell's DNA is the organism's DNA the cell has processes of self-destruction to meet needs of the developing and living organism. Many pathways of cell metabolic malfunction start a process called apoptosis, another metabolic pathway through which a cell destroys and detoxifies itself to preserve the organism.

The organism has systems that can recognize “bad” cells and eliminate these when neighbor cells induce apoptosis, for example, in a cell or cell type not needed at the moment or in non-productive cells. In ideal normal circumstances, the organism's immune system can act as a back-up to recognize and help eliminate improperly functioning cells.

The elegance of life is that, although imperfections occur, organisms have at their disposal multiple defenses to correct, overcome, minimize or eliminate metabolic shortcomings. Taking advantage of the tools the cells and the organisms have in their repertoire can effectively eliminate or counteract developed defects in metabolism.

### BRIEF DESCRIPTION

Many diseases result not from one single metabolic mistake but evolve from a compounding of several events that eventually manifest as a disease state. For example, deceased vigor with age, Alzheimer's disease, cancers, several auto-immune diseases, and many progressive diseases involve multiple mutational and/or compensatory events for the cell's survival. From the cell's perspective, one metabolic change will affect all downstream pathways, some of which will involve feedback loops to one or more upstream paths. The initial event and compensatory responses may tilt the selective pressures such that events we might normally count as mistakes might in fact, from the cells position, merely be the most opportunistic response to improve continuity of that cell and its lineage. A short-term advantage to a cell may often prove to be deleterious to the cell or organism in the long run.

Modifying selective pressures to disfavor these compensatory secondary or tertiary modifications that are at the moment advantageous to the modified cell, but not to the organism's long-term well-being, can prevent progression to or progression along a disease state. Recognizing the early “errors” and engaging the cell's or organism's compensatory mechanisms is a preferred and natural path for preventing or eliminating metabolic or proliferative disease or heightened risk of disease.

Since metabolism is complex and comprises many different metabolic pathways that might, in response to momentary stress circumstance, maladapt and thereby lead to cell-survival-enhancing, but organism-degrading, opportunistic compensatory maladaptation, a large number of outside interventions are available as tools to rebalance the cells towards more long-term organism and less immediate cellular benefit. Many of the second, third, fourth, . . . , reactions will to some extent lessen the impact of the first maladaptation, for example, by providing less substrate (e.g., LeChatelier's feedback) when a the maladapted path less vigorously consumes a substrate; by activating a parallel, crossing or serial path when a product becomes in excess or an intermediate product is released; or up-regulating or down-regulating through another process, or e.g., through a more complex process perhaps involving stabilizing or catabolizing a protein, altering RNA metabolism, and/or activating or deactivating transcription factor pathways.

Pathways that may be advantageously strengthened, redirected or co-opted include, but are not limited to: energy pathways (for example, pyruvate producing, ox-redox reactions, ATP or other energetic phosphate producing, fatty acid breakdown and synthesis, sugars metabolism), phosphorus metabolism, ubiquitination/deubiquitination, transition metal control, OXPHOS—aerobic glycolytic balance, uric acid metabolism, purine and pyrimidine metabolism, etc., many of which are discussed below. These systems and others may become maladapted, but all might be modified to retilt the cell's and or organism's corrective tools towards preventing further maladaptive events and preferably to reverse, impede or eliminate an initial precipitating event or early level compensatory adaptations.

Our metabolisms are complex with multiple reactive pathways that generally support continued life. It is not just our metabolism, but we now recognize that we have a commensal and sometimes synergistic relation with other organisms, especially our various microbiomes. Our skin microbiome helps determine which chemicals might cross our dermis. Microbiota in our mouths and nasal passages act as defenses against pathogens—but also begin processing what we eat or breathe in. Our GI tract has multiple zones, each with its specialized microbe populations. Our microbiome has almost certainly changed as: a) civilization has altered our atmosphere and the houses and cities in which we live, b) our food sources have evolved, c) sanitary practices changed microbes, metals, and toxins in our diets, and d) use of antibiotics became more prevalent. Our DNA may not have fully adapted to relatively recent developments. E.g., fewer than twenty generations have had opportunity to genomicly adapt to major changes including, but not limited to: indoor plumbing, sanitary sewers, central heat, central air conditioning, refrigeration, industrial farming, prepared foods, microwave cooking, germ theory and antibiotics, antivirals, the automobile, the industrial age, synthetics, air pollution, water pollution, anesthesia, NSAIDS, survivable surgery, useful diagnostic tests, etc.

The ability of our genomic material to provide life sustaining metabolic support in the face of these rapid significant changes speaks to the robustness of our systems. Our cells adapt their metabolisms to respond to the preceding reactions in the cell. Cells adapt by means of up-regulating and down-regulating specific pathways in ways responding to the new circumstance at any instant. But cells of our microbiomes have cycled through many more generations during our individual lifetimes. Each microbe will have responsive metabolism adapting to each of its internal reactions, but also in response to the environment created by the adapting host organism. During these multiple generations microbes will have, as part of their adaptive abilities, exchanged genetic material with other microbes allowing for profound and lasting adaptations. Our bodies—and the microbes inhabiting our bodies—may have received minimal changed instructions from our genetic material (including mutations and epigenetic modification) but even in the face of these changed instructions life depends on a series of metabolic reaction events interacting through time in series and in parallel.

As metabolism progresses, one pathway may begin to switch towards an opportunistic imbalanced state. Other pathways, when compensating for or reacting in response to products of the imbalanced state, will have their conventional activity refashioned in accordance with the switched circumstance. Absent corrective intervention cells' metabolisms will continue to drift.

Even when no mutation has been prompted in the nuclear or mitochondrial DNA there will be differences in activation and expression of metabolic genes. Active DNA, RNA and proteins will differ from the original efficiently progressing metabolism. Products resulting from the switched metabolism will serve as markers of the switching metabolisms.

Energy Metabolism Overview

Carbohydrates (sugars) are the primary fuel for producing usable chemical energy in our cells. In the big picture sugars enter the cell and are converted to glucose-6-phosphate (G6P) and then to pyruvic acid in the cytoplasm. Pyruvic acid can form lactic acid or can convert to acetyl CoA to enter the citric acid cycle and electron transport chain in the mitochondria. Acetyl CoA can be diverted for synthesizing lipids and can also be obtained from breaking down lipids or glycerol. G6P can be diverted to produce the amino acid, glycine and sequelae.

Metabolism, in essence, includes processes to provide the structure and mechanics to support the life of the organism. Especially significant paths include but are not limited to: glycolysis, the Krebs or citric acid cycle, ketogenesis, fatty acid synthesis, the urea cycle, the hexose monophosphate shunt, membrane transport, transcription, translation, protein expression, component assemble and recycling, repair mechanisms, transport, etc.

Switching the multitude of processes on and off at appropriate times for optimal benefit to the organism is a complex challenge which, though the organism and cells of the organism are adept at accomplishing, on occasion may take on sub-optimal or even detrimental paths. The tremendous number of reactions and their interactions mean that even a when only an extremely minute fraction of the activities are suboptimal the sheer number of required activities means that metabolic errors will occur in meaningful quantity.

For example, in animals, glycogen converts to glucose-1-phosphate then to glucose-6-phosphate which may generate glucose, 6-β-gluconolactone or fructose-6-phosphate. The fructose 6-phosphate can process to fructose-1,6-bisphosphate and then to phosphoenolpyruvate and pyruvate. Pyruvate can process to lactate, oxaloacetate or acetyl-CoA. Acetyl-CoA can enter the citric acid cycle for ATP generation or other synthesis processes, may process to acetoacetate, then β-hydroxybutyrate for ketogenesis, or may process to malonyl-CoA for fatty acid synthesis. The 6-β-gluconolactone is used to produce the ribose sugars necessary for nucleic acid synthesis or can process through glycolysis to pyruvate. The many processes are complex in themselves with multiple steps and multiple branch points any or which might prove sub-optimal on small occasions. These branch points have multiple interactions and parallel paths that may provide means for restore proper metabolism or may themselves cause, maintain or exacerbate the earlier sub-optimal activity.

Thyroid hormone, palmitic acid, and even light activate a crucial path that suppresses the formation of lactic acid. Palmitic acid acts as an antioxidant with capacity to regenerate other antioxidants such as vitamins C, E, and glutathione. Lipoic acid also participates in recycling CoQ10 and NAD.

Palmitic acid occurs in simple foods like coconut oil, whose consumption may up-regulate and/or down-regulate multiple related pathways.

Breakdown products of proteins can feed into the energy metabolism paths at pyruvic acid, acetyl CoA or the citric acid cycle. The citric acid cycle can feed or feed off the urea cycle, a means leading to excretion of nitrogen from proteins' amino groups when carbon atoms are harvested for other outcomes. Malonate, an inhibitor of the citric acid cycle, can be consumed for fatty acid synthesis by the mitochondria. The mtFASII pathway synthesizes fatty acids with acyl chains of at least 14 carbons long (myristic acid). One recognized destination of mtFASII products results in the creation of lipoic acid. To create lipoic acid, lipoyl synthase uses octanoic acid from the mtFASII pathway and S-adenosyl methionine. Lipoic acid is a cofactor for many enzymes, including pyruvate dehydrogenase, α-ketoglutarate dehydrogenase, and the branched chain oxoacid dehydrogenase. Therefore, knockdown of mtFASII components results in reduced citric acid cycle metabolism and reduced cellular lipoic acid content with resultant reduction of protein lipoylation levels.

The cytoplasm carries out the glycolytic early stages with the general equation: C₆H₁₂O₆+2 NAD⁺+2 ADP+2P----->2 pyruvic acid (CH₃(C═O)COOH)+2 ATP+2 NADH+2H⁺.

Glycolysis can utilize multiple inputs. For example, glycogen, glucose and galactose can be phosphorylated by ATP to ADP conversion to make the G6P. G6P can be converted to Fructose-6-phosphate (F6P) or fructose can be directly phosphorylated to make F6P. Another phosphate is added to form fructose-1,6-diphosphate (F1-6P) which then makes two glyceraldehyde-3-phosphate (G3P) molecules. NAD⁺ then oxidizes G3P to 1,3-diphosphoglycerate (1-3DPG). 1-3DPG then produces an ATP as it dephosphorylates to form 3-phosphoglyceric acid (3PG) and then phosphoenolpyruvic acid (PPA) which is dephosphorylated to produce pyruvic acid and another ATP. The pyruvic acid can convert to lactic acid or enter the citric acid cycle.

2 ATP molecules were needed to make the F1-6P. Then each F1-6P makes 2 G3Ps each of which generates 2 ATPs to give a net gain of 2 ATP molecules for every glucose or fructose consumed.

Inhibition of succinate oxidation by malonate is a recognized switching phenomenon since the oxidation of succinate to fumarate is an integral part of the Krebs or citric acid cycle. It has been generally recognized that the inhibitory effect of malonate upon the oxidation of any member of the cycle results from the inhibition of the succinate to fumarate step. In addition to the inhibition resulting from a block of succinate oxidation, malonate inhibits the citric acid cycle through at least a second mechanism that is Mg dependent.

Four apparent regulatory enzyme steps are catalyzed by hexokinase, glucokinase, phosphofructokinase, and pyruvate kinase. The rate of the glycolytic pathway is adjusted in response to intracellular and extracellular circumstance. The intracellular factors that regulate glycolysis tend to upregulate or downregulate activity such that ATP is produced to meet the cell's needs. Extracellular circumstances are usually controlled by circulation, hormones, and nutrition availability.

Phosphorylation by kinase enzymes is a common means for controlling enzymatic activities. Kinases can be responsive to hormones, other kinases, ions or intracellular events. Kinases modulate metabolic activity by catalyzing phosphate binding at specific sites. Hexokinase and glucokinase activities are controlled by intracellular G6P and blood glucose concentrations, respectively, independent of direct hormonal modulation. Phosphofructokinase is another important gate point in the glycolytic pathway, since it is irreversible and has allosteric effectors, AMP and fructose 2,6-bisphosphate (F2,6BP).

When glucose has been converted into G6P by hexokinase or glucokinase, it can either be converted to glucose-1-phosphate (G1P) for conversion to glycogen, or it is alternatively converted by glycolysis to pyruvate, which enters the mitochondrion where it is converted into acetyl-CoA and then into citrate. Excess citrate is exported from the mitochondrion back into the cytosol, where ATP citrate lyase regenerates acetyl-CoA and oxaloacetate (OAA). The acetyl-CoA is then used for fatty acid synthesis and cholesterol synthesis, two important ways of utilizing excess glucose when its concentration is high in blood. The rate limiting enzymes catalyzing these reactions perform these functions when they have been dephosphorylated, for example, through the action of insulin on the liver cells.

Cholesterol is important as a source of steroid hormones produced, for example, in adrenal gland and gonads. Steroid hormones, especially the sex hormones, exhibit different influences depending on gender and other active pathways. Synthesis within the body is tissue dependent, for example in females, 25% of testosterone is ovarian and 25% is adrenal—with the remainder produced by a broad collection of cells. But more important is conversion of testosterone to dihydrotestosterone which occurs intracellularly to activate dihydrotestosterone. As an intracellular messenger that can increase protein kinase A (PKA), intracellular Ca, protein kinase C (PKC), c-Sirc (sometimes in concert with palmitate) and MAPK pathway proteins. The associated release of intracellular Ca is an apoptosis promoter. These intracellular messenger activities of dihydrotestosterone and similarly acting cholesterol derivatives are independent of the classic steroid pathway involving transport into the nucleus and stimulating transcription.

Between meals, during fasting, exercise or hypoglycemia, glucagon and epinephrine are released into the blood. This causes liver glycogen to be converted back to G6P, and then converted to glucose by the liver-specific enzyme, glucose 6-phosphatase, and released into the blood. Glucagon and epinephrine also stimulate gluconeogenesis, which coverts non-carbohydrate substrates into G6P, which joins the G6P derived from glycogen, or substitutes for it when the liver glycogen store have been depleted. This conversion is critical for brain function, since the brain utilizes glucose as an energy source under most conditions. The simultaneously phosphorylation of, particularly, phosphofructokinase, but also, to a certain extent pyruvate kinase, prevents glycolysis occurring at the same time as gluconeogenesis and glycogenolysis.

All cells contain the enzyme hexokinase, which catalyzes the conversion of glucose that has entered the cell into glucose-6-phosphate (G6P). Since the cell membrane is impervious to G6P, hexokinase essentially acts to transport glucose into the cells from which it can then no longer escape. Hexokinase is inhibited by high levels of G6P in the cell. Thus, the rate of entry of glucose into cells partially depends on how fast G6P can be disposed of by glycolysis, and by glycogen synthesis (in the cells which store glycogen, namely liver and muscles.

Glucokinase, unlike hexokinase, is not inhibited by G6P. It is especially active in liver cells, and will only phosphorylate the glucose entering the cell to form glucose-6-phosphate (G6P), when the sugar in the blood is abundant. This being the first step in the glycolytic pathway in the liver, it therefore imparts an additional layer of control of the glycolytic pathway in this organ.

Phosphofructokinase

A primitive energy source still present in today's cells embodies pyrophosphate (PPᵢ). PPᵢ is released with each nucleotide polymerized into a DNA or RNA. PPᵢ is highly anionic with a (−)4 charge, but in aqueous environment of the cell pyrophosphatases (PPase) hydrolyze PPᵢ to dihydrogen phosphate ion (H₂PO₄⁻²). Thiamine is a cotransport molecule for moving PPᵢ mitochondrial membranes.

PPᵢ, as a charged particle, is not transported efficiently across cell membranes. To prevent the product PPᵢ from slowing the reactions producing it (LeChatelier's principle), PPᵢ must be removed from its intracellular sources. The family of PPases is found in both prokaryotic and eukaryotic cells. For example, PPA2 appears necessary for mitochondrial DNA (mtDNA) maintenance in several species. Mitochondrial PPases have a close spatial relationship with IMM proteins, especially components of the respiratory chain PPase2 has been successfully targeted with siRNA.

The antioxidant function of glutathione (GSH) is accomplished largely by GSH peroxidase (GPx) catalyzed reactions, which reduce hydrogen peroxide and lipid peroxide as GSH is oxidized to GSSG. GSSG in turn is reduced back to GSH by GSSG reductase at the expense of NADPH, forming a redox cycle. Organic peroxides can also be reduced by GPx and GSH S-transferase. Catalase can also reduce H₂O₂, but it is present only in peroxisome, another organelle. This makes GSH particularly important in the mitochondria for defending against both physiologically and pathologically generated oxidative stress. As GSH to GSSG ratio largely determines the intracellular redox potential (proportional to the log of [GSH]²/[GSSG]), to prevent a major shift in the redox equilibrium when oxidative stress overcomes the ability of the cell to reduce GSSG to GSH, GSSG can be actively exported out of the cell or react with a protein sulfhydryl group leading to the formation of a mixed disulfide. Thus, severe oxidative stress depletes cellular GSH.

Amino Acids

Proteins form structural cell components, participate in intracellular transport, act as receptors and transmembrane channels or carriers, carry information as hormones, and catalyze most reactions of metabolism. Proteins are the most predominant molecule in the body, second only to H₂O. Proteins are polymeric assemblies of amino acids.

Proteins are polypeptide chains, polymers of amino acids linked though peptide bonds. The human uses 20 different amino acids in the genetic code for its proteome, each amino acid varying from others in its characteristics including, but not limited to: size, H⁺ ion binding characteristics, hydrophobicity, its tRNA(s), interaction with other amino acids and substrates, other proteins or signal molecules, and reactive sites. Phenylalanine, leucine, isoleucine methionine valine, proline, alanine and tryptophan are hydrophobic and tend to avoid water; serine, threonine tyrosine, histidine, glutamine, glutamic acid, asparagine, aspartic acid, lysine, cysteine, arginine and glycine are polar—like water. The acids are acidic, while arginine, lysine and histidine are basic. Hydroxyproline is post translationally modified and N-formylmethionine is a methionine form found as an initiation amino acid in mitochondrial protein synthesis.

Side groups of the amino acids determine their binding activities. Polar side groups tend to face the aqueous environment and thus are accessible to products for enzymatic reactions. Reactive side groups, those whose charge is mutable, including, but not limited to: arginine, threonine, serine, glutamine, cysteine, methionine, aspartic acid, glutamic acid, lysine, histidine, tryptophan and proline are especially involved in catalysis and transport. Non-polar amino acids are generally involved in establishing folding stability and other 3-dimensional structures.

Non-protein compounds including, but not limited to: carnitine and porphyrins are derived from amino acids—and amino acids can provide carbons for other molecules such as glucose during gluconeogenesis. Most amino acids can be converted into oxaloacetate and subsequently into pyruvate to enter the gluconeogenic pathway or consumed as chemical energy. Only leucine and lysine cannot follow this path. Alanine, cysteine, glycine, serine, threonine and tryptophan can convert to pyruvate which then can take its own path through acetyl-CoA, lactate, etc. These can feed through the citric acid cycle to oxaloacetate for degradation to glucose. Arginine, glutamine, glutamic acid, histidine and proline can enter the citric acid cycle as α-ketoglutarate and be processed to oxaloacetate. Isoleucine, methionine, and valine can enter the cycle as succinyl-CoA and aspartic acid, phenylalanine and tyrosine can enter at fumarate for processing to oxaloacetate. Asparagine and aspartic acid can also enter at oxaloacetate. The citric acid cycle thus can be co-opted for gluconeogenesis from amino acids when metabolic needs require.

Ketogenic amino acids, leucine, lysine, phenylalanine, tryptophan and tyrosine can convert to acetoacetate. Resultant acetoacetate and the amino acids, isoleucine, leucine, lysine and threonine can enter the citric acid cycle as acetyl-CoA and progress through to oxaloacetate for gluconeogenesis.

Amino acids are organic carboxylic acid compounds with an amine group —NH₂, on the α-carbon and the carboxyl group —COOH on the terminal carbon. “Side chains”, The “R” group on the α-carbon, define the amino acid and provide its chemical characteristics. Every amino acid comprises carbon, hydrogen, oxygen and nitrogen, and sulphur is present in methionine and cysteine. In humans all stereo active amino acids (those with an asymmetric carbon) are the L-stereoisomer.

Amino acids are essential for cell growth and proliferation because they are the building blocks for protein, the activity centers of the cell. Protein synthesis, like other enzymatic activities within the cell, requires energy in the form of ATP. Multitudinous enzymes act in concert to produce ATP for the cell. Mitochondria are energy producing organelles that make most cell ATP, comprise multiple membrane complexes and other transport and catalytic structures and play a central role in amino acid homeostasis. Humans do not have metabolic pathways to make the protein building block amino acids: phenylalanine, valine, threonine, tryptophan, methionine, leucine, isoleucine, lysine, and histidine. These must be obtained from sources outside the body (food) and delivered by the gut and circulatory system in adequate quantities to the cells.

Amino acids, essential or otherwise, are absorbed through the intestinal wall obtaining energy from Na⁺ or H⁺ cotransport. Identical or analogous transporters move amino acids across cellular membranes. Short protein fragments, polypeptides (amino acid polymers) up to about 6 amino acid residues in length can be absorbed through these systems. Six major families of transporters have been characterized. Diacidic, dibasic (including cysteine) and neutral amino acids are considered separate categories in the six gene families: SLC1, SLC6, SLC7, SLC36, SLC38, and SLC43. Different subfamily members express preference for one or more amino acid or amino acid residue.

The neutral amino acids: glycine, proline, valine, alanine and citrulline can cross the inner mitochondrial membrane (IMM) without significant energy expended for their transport. Citrulline is not encoded in the DNA but is produced by post-translational processing from arginine.

The transporters of amino acids may serve as important metabolic signals. As suggested by Peter Taylor in Role of amino acid transporters in amino acid sensing:
Amino acid (AA) transporters may act as sensors, as well as carriers, of tissue nutrient supplies. This review considers recent advances in our understanding of the AA-sensing functions of AA transporters in both epithelial and nonepithelial cells. These transporters mediate AA exchanges between extracellular and intracellular fluid compartments, delivering substrates to intracellular AA sensors. AA transporters on endosomal (e.g., lysosomal) membranes may themselves function as intracellular AA sensors. AA transporters at the cell surface, particularly those for large neutral AAs such as leucine, interact functionally with intracellular nutrient-signaling pathways that regulate metabolism: for example, the mammalian target of rapamycin complex 1 (mTORC1) pathway, which promotes cell growth, and the general control non-derepressible (GCN) pathway, which is activated by AA starvation. Under some circumstances, upregulation of AA transporter expression [notably a leucine transporter, solute carrier 7A5 (SLC7A5)] is required to initiate AA-dependent activation of the mTORC1 pathway. Certain AA transporters may have dual receptor-transporter functions, operating as “transceptors” to sense extracellular (or intracellular) AA availability upstream of intracellular signaling pathways. New opportunities for nutritional therapy may include targeting of AA transporters (or mechanisms that upregulate their expression) to promote protein-anabolic signals for retention or recovery of lean tissue mass.

Amino acid transport is coupled to other components that cross membranes, especially ions such as Na⁺, K⁺, and H⁺ that are actively pumped and common anions like Cl⁻. Taylor suggests several signal pathways of relevance to mammalian metabolism:
The major AA sensing-signaling pathways in mammalian cells are the mammalian target of rapamycin complex 1 (mTORC1) and general control non-derepressible (GCN) pathways. The AA-sensing mechanisms of the mTORC1 pathway, which is activated when certain AAs (e.g., leucine) are abundant, appear to involve monitoring AA concentrations in both cytosol and subcellular organelles such as lysosomes. The GCN pathway primarily senses intracellular AA availability at the level of AA “charging” on transfer RNA (tRNA) bound to the GCN2 protein kinase and is activated when one or more AAs are scarce. AA transporters have important roles upstream and downstream of both mTORC1 and GCN pathways and may help in monitoring both intracellular and extracellular AA abundances. AA transporters may act directly as the initiating sensor for a signaling pathway—for example, activation of mTORC1 signaling by the SLC38A2 transporter—or may serve as a conduit for delivery of AAs to intracellular sensing pathways, notably the leucine transporter SLC7A5 for mTORC1 activation. AA transporters may also generate indirect nutrient-related signals related to effects of cotransported solutes on intracellular pH and volume. [References omitted.]

Thus, amino acids and pathways related to amino acid signaling can serve as valuable target switch points in correcting metabolic digression. Compounds that may be used to modulate amino acid availability to the cell include, but are not limited to: d-amino acids, d-alanine, d-cysteine, d-aspartic acid, d-glutamic acid, d-phenylalanine, d-histidine, d-isoleucine, d-lysine, d-methionine, d-asparagine, d-proline, d-glutamine, d-arginine, d-serine, d-threonine, d-valine, d-tryptophan, d-tyrosine, threo-β-hydroxyaspartate, dihydrokainate, threo-β-benzyloxyaspartate, etc. Even absent such intervention, the human metabolism is constantly changing. Each (biochemical) reaction occurs in an environment with multiple responsive reactions and their sequelae.

Analogous to the notion that no two humans, even identical twins, are identical and indistinguishable, no two cells will exactly mirror the environment of any other cell. No two cells can have identical neighbors. No two cells can have the same nutrient availability. No two cells will have identical response to an outside event. Similarly, since no chemical reaction occurs in isolation, even if we know the location, substrate and products, we cannot accurately predict every downstream responsive event. An arbitrary “first” biochemical reaction will consume a substrate that might have been put to another enzyme's use and will release at least one product to act on or be reacted with another molecule in the cell. Feedback loops within the cell will control rates of reactions. Chemical or biochemical compounds may induce expression of pathways to eliminate or take advantage of them. Induced pathways may and often do interact with many sequential, parallel or crossing pathways. Each cell is an unpredictable dynamo—except each cell has a gene pool which restricts its possibilities and each cell and each transporter or catalyst within the cell has to work within the limits of its environment—with respect to temperature, available substrates, cofactors, products (for reversible reactions), electrochemical status, etc.

So, this first reaction produces a product that will be acted on by other actors within the cell. That first reaction had opportunity costs. It consumed a product that might otherwise have been available to another actor. Each actor is restricted by its individual circumstance and its actions will contribute to setting circumstances of other actors. Each actor involved will act in accordance with its limits and circumstance and will, by this action, opportunistically set in place new circumstance for subsequent actors. Essentially, the cell with each reaction sets the stage for its future events. These events will be defined by the circumstance when each occurs. The second, third, fourth, etc., biochemical reactions will be responsive to earlier reactions. When these reactions cause a cell to switch its balance to metabolic pathways that stray from efficient metabolisms, some intervention, perhaps including cell death initiated by cell or organism may be called upon to restore proper metabolic balance. The farther a cell's metabolism has drifted from optimal, the more intense the rebalancing intervention will need to be. Early on in the cells switching to sub-optimal metabolic pathways, imbalance will be less severe and rebalancing intervention can succeed without resorting to drastic and expensive interventions.

Essential Amino Acids

As mentioned above, the human genome has not provided pathways for making all the amino acids. Our foods must supply these in the diet. Histidine, isoleucine, leucine, lysine, methionine, phenylalanine, threonine, tryptophan, and valine are considered essential for a diet to support proper health. Carriers for these as well as the other amino acids are important control functions in metabolism.

Glutamine

Glutamine is the most common circulating amino acid. Its interconversion with glucose for energy production and its ability to provide carbon for fatty acid synthesis make glutamine availability essential for long term cell survival. The nitrogen group of glutamine is also important as a source in purine synthesis. Glutamine is transported into mitochondria through a pH dependent carrier exchanging a proton (H⁺) for glutamine. In the mitochondrion, ribose-5-phosphate (a product whose synthesis consumes a G6P and produces 2 NADPH) de-energizes an ATP to AMP when acted on by PRPP synthase (a Mg-dependent enzyme) to 5-phosphoribosyl-1-phosphate (PRPP). PRPP is activated to supply the ribose sugar for de novo synthesis of purines and pyrimidines, essential components in the nucleotide bases that form RNA and DNA. PRPP synthetase is activated by phosphate and inhibited by purine nucleotides. PRPP-amidotransferase then converts glutamine to glutamate using the A-amine group to make 5-phosphoribosyl amine. After PRPP adds the amine to the ribose ring, a glycine is added followed by N¹⁰-formyl-THF and nitrogen from glutamine. An aspartate is added and a fumarate expelled by adenylosuccinate lyase. And another N¹⁰-formyl-THF carbon is added. Finally, inosinemonophosphate (IMP) (precursor of ATP and GTP, components for RNA and DNA synthesis) is made. Enzymes in the pathway include ribose phosphopyrokinase, amidophosphoribosyl transferase, GAR synthase, GAR transtransformylase, FGAM synthase, AIR synthase, AIR carboxylase, SAICAR synthetase, adenylosuccinate lyase, AICAR transformylase and IMP cyclohydrolase. Other pathways involving PRPP as a substrate include, but are not limited to those that produce: NAD, NADP, histidine, tryptophan, etc.

IκB kinase β inhibits 6-phosphofructo-2-kinase and thereby slows the glucose consumption by ETC and causes acidification through increased production of lactic acid. Under these conditions glutamine and its product glutamate become a coveted nutrient for maintenance of α-ketoglutarate and GSH levels in mitochondria.

Glutamine can be synthesized from glutamate and ammonia by glutamine synthase. Muscles are the predominant supplier of circulating glutamine. Production of the glutamate substrate diverts α-ketoglutarate from ATP production to form α-keto acid and glutamate. The glutamine synthetase phosphorylates the Δ-carbon activating it for adding the Δ-amine to synthesize the glutamine. Amino acids are a source of amine groups for α-ketoglutarate. Amino acids: alanine, serine, threonine, histidine and tryptophan are inhibitors of glutamine synthesis. Two of these, histidine and tryptophan are made from glutamine. Carbamoyl phosphate, glucosamine-6-phosphate, AMP and CTP, products of glutamine consumption also inhibit glutamine synthesis.

Glutamine, Glutamate, Alanine, Asparagine and Aspartate

Glutamine is the predominant amino acid in circulation. Glutamine is readily converted to glutamate and aspartate, the anion part of the acidic amino acids present as ions in aqueous solutions—and then to alanine. Glutamate itself can act as a neurotransmitter.

Glutamine serves as source molecule to produce citrate, pyruvate and lactate. Glutamine is also a source for lipid synthesis and N for purine metabolism.

Glutamate is obtained when glutamine is hydrolyzed by glutaminases in several locations to release NH₃ which becomes ammonium (NH₄⁺) in aqueous environments. ADP is a strong activator of mitochondrial glutaminase, while ROS species are inhibitory. Glutamate is a reactant for glutamate dehydrogenase, alanine transaminase and aspartate transaminase.

Asparaginase's conversion of asparagine to aspartate is one means of shutting off protein synthesis. The ribosomal polymerization will stop when, for example, it is not occupied by an arginine bound tRNA. Not only is that protein's production halted, but the ribosome is blocked from synthesizing other proteins. Asparaginase produces ammonia and aspartate from asparagine.

Aspartic acid is the name for protonated form of one of the amino acid residues used in protein synthesis. At normal body pH, near neutral, most free aspartate disassociates into H⁺ and aspartate. Asparagine can be hydrolyzed to form aspartic acid. Thus, aspartic acid can be considered to be a spontaneous producer of aspartate because producer of asparate, in this case because of the association-dissociation equilibrium. In some cases, a prodrug will spontaneously produce an active substance by isomerization, enzymatic action or other chemically favored reaction. The prodrug Seldane™ or terfenadine spontaneously became the active drug Allegra™ or fexofenadine when metabolized by CYP3A4 in the liver.

Aspartic acid is also synthesized from glutamate and oxaloacetate.

Aspartate is an important participant in the malate/aspartate shuttle. Shuttles are an important regulator of metabolism in eukaryotic cells because most metabolic processes occur in specific compartments within the cell. Separate pools of some important metabolites are made, transported and stored in various different locations. Controlling movement of the substrate or enzyme molecules between compartments is a significant form of metabolic regulation or a serious problem for the cell when shuttling is awry. This compartmentalization is especially relevant for mitochondria, where the inner membrane is a barrier to the movement of most molecules whether electrically charged or neutral.

Alanine transaminase converts glutamate and pyruvate to α-ketoglutarate and alanine, respectively. Aspartate transaminase is a bi-directional enzyme interconverting aspartate and α-ketoglutarate between oxaloacetate and glutamate.

Pyruvate, as an alternative to entering the ETC or producing lactate, can be acted on by alanine transaminase to convert glutamate to 2-oxoglutarate and produce alanine.

Arginine

Arginine is synthesized from citrulline in the arginine/proline metabolism by the sequential action of the cytosolic enzymes argininosuccinate synthetase (ASS) and argininosuccinate lyase (ASL). The pathways linking arginine, glutamine, and proline are bidirectional. So, for example, citrulline can be a source or product of alanine. Arginine is active at catalytic sites and is especially essential in cell division and wound healing.

Histidine

Histidine is a slightly basic amino acid because its imidazole side chain has affinity for H⁺. Its pKₐ is 6.0 which means that slight changes in proton concentration will change histidine's charge. This pH sensitivity renders histidine a frequent participant in active sites of enzymes and carriers. The chemistry of the imidazole ring of histidine makes it a nucleophile and a good acid/base catalyzer. Histidine often participates with hydroxyl group containing threonine or serine or with the sulfhydryl of cysteine in moving hydrogens.

Cysteine and Methionine

The sulfur containing amino acids, cysteine and methionine, in humans are dependent on adequate intake of methionine, one of our essential amino acids. Methionine adenosyltransferase (MAT) converts methionine to S-adenosylmethionine (SAM). SAM is a precursor used for other compounds such as for conversion of norepinephrine to epinephrine. S-adenosylhomocysteine is then cleaved by adenosylhomocyteinase to produce homocysteine and adenosine. Homocysteine then condenses with serine to form cystathionine which is then catalyzed by cystathionine β-synthase. Cystathionine is subsequently cleaved by cystathionine γ-lyase to produce cysteine and α-ketobutyrate. The sum of the latter two reactions is known as transsulfuration. The sulfur atom in these amino acids participates in electron transport.

Vitamin B₁₂ is an essential cofactor for many methionine-based reactions.

Together with cysteine, methionine is one of two sulfur-containing proteinogenic amino acids. Excluding the few exceptions where methionine may act as a redox sensor, methionine residues do not generally have a catalytic role in enzymatic activity. But cysteine residues, contribute a thiol group as a catalytic intermediate in many protein reactions.

These sulfur containing amino acids are also essential in coordinating synthesis and maintenance of iron-sulfur (Fe—S) complexes and their electron transport activities for catalysis.

Tyrosine and Phenylalanine

The essential amino acid, phenylalanine, is the source of tyrosine, similar to the relationship between methionine and cysteine. Phenylalanine hydroxylase catalyzes the conversion. Deficiencies in this enzyme result in PKU, phenylketonuria. Tyrosine is especially active in neurotransmission.

Proline

Proline uses glutamate as its precursor. Proline is a folded amino acid important for protein two and three-dimensional structure. Glutamate is acted on by Δ-1-pyrroline-5-carboxylate synthase to make glutamyl-γ-phosphate as an intermediate for Δ-1-pyrroline-5-carboxylate. Then pyrroline-5-carboxylate reductase 1 uses either NAD⁺ or NADP⁺ to form proline.

Serine

A major serine biosynthesis pathway starts with the glycolytic intermediate 3PG, diverted from pyruvate formation. Then 3-phosphoglycerate dehydrogenase converts 3-phosphoglycerate to 3-phosphohydroxypyruvate which is capable of transamination. Phosphoserine aminotransferase 1, with glutamate makes 3-phosphoserine, which is converted to serine by phosphoserine phosphatase. Serine can also be interconverted with glycine in a single step reaction with serine hydroxymethyltransferase (SHMT) and tetrahydrofolate (THF). This interconversion of serine and glycine using THF represents the major pathway for the generation of N5,N10-methylene-THF, an intermediate required for purine nucleotide and thymine nucleotide biosynthesis.

Humans express two serine hydroxymethyltransferase genes: a cytosolic enzyme and one located in the mitochondria. One of the major functions of the SHMT2 encoded enzyme is in mitochondrial thymidylate synthesis pathway via its role in glycine and tetrahydrofolate metabolism. Mitochondrial thymidylate synthesis is required to prevent uracil accumulation in mitochondrial DNA (mtDNA).

Serine is also used to make cysteine from the methionine metabolite, homocysteine.

Glycine

The main pathway to glycine is a one-step reversible reaction catalyzed by serine hydroxymethyltransferase (SHMT). Glycine is the smallest of the amino acids and the only one without optical activity because it lacks an asymmetric carbon. Glycine is not often essential in catalysis since it lacks a reactive side group.

Iron Sulfur Complexes

Iron-sulfur (Fe—S) clusters are omnipresent cofactors that take advantage of the variable oxidation states of iron and inorganic sulfur. The variable oxidation states are useful for protein activities in a wide range of functions, for example, electron transport in respiratory chain complexes, regulatory sensing, DNA repair and, in plants, photosynthesis. The proteins responsible for biogenesis of Fe—S clusters are evolutionarily conserved from archaic life forms up through to modern bacteria and to humans.

Fe—S clusters are important prosthetic groups with special chemical properties that enable the proteins associated with them (Fe—S proteins) to function in diverse pathways ranging throughout metabolism. Most Fe—S proteins are evolutionarily ancient and today are present in essentially all organisms, including archaea, bacteria, plants and animals. This high level of evolutionary conservation is consistent with the belief that Fe—S clusters contributed to the success of early life forms and that activity of Fe—S clusters and Fe—S proteins are a basic requirement for life on earth. A significant number of DNA repair enzymes are Fe—S proteins—including the protein responsible for excision-repair of UV damage.

Fe—S clusters as cofactors are generally ligated to the cysteine residues of proteins, where they can facilitate numerous types of reactions. The most common form is as a cubane that contains four Fe and four inorganic S atoms. These Fe—S clusters are extraordinarily chemically versatile taking advantage of both Fe and S to readily donate or accept multiple electrons. The chemical versatility supports features that allow the electron affinity of each Fe—S cluster to be fine-tuned across an extremely broad electrochemical range that is dependent on the surrounding amino acid residues in that Fe—S protein. For example, in mitochondrial complex I, there are seven Fe—S clusters with gradually increasing reduction potentials that are configured to form a wire-like conductive pathway for the electrons ascend. Thus, this varied ability of Fe—S clusters to maintain low reduction potentials (i.e. low affinity for electrons) allows the highly efficient capture of chemical energy from NADH from electrons moving progressively through the respiratory chain complexes.

Fe—S clusters are versatile in other ways. They directly facilitate chemical reactions by binding to an Fe—S protein's substrate, for example in the aconitase portion of the citric acid cycle, where the enzyme interconverts citrate and isocitrate. Fe—S proteins also function as sensors in bacteria and eukaryotes. Bacterial FNR and IscR proteins are Fe—S proteins as is IRP1, an Fe—S protein that regulates cytosolic iron metabolism in mammals. Fe—S proteins are vigorously active players in multiple subcellular compartments, including, but not limited to: mitochondria, plastids, cytosol and nucleus.

Biogenesis of Fe—S clusters in mammalian cells follows a common general paradigm. NFS1, a cysteine desulfurase, dimerizes to bind monomers of the primary scaffold protein ISCU. In eukaryotes, ISD11 is an obligate binding partner for NFS1 and NFS1 also binds the cofactor pyridoxal phosphate. Frataxin is associated with the initial Fe—S cluster biogenesis complex physically between NFS1 and ISCU. NFS1 provides the inorganic S and ISCU cysteines provide S ligands that directly bind Fe in the nascent Fe—S cluster. A highly reduced protein such as ferredoxin then provides needed electrons.

After the Fe—S cluster is assembled, a short, conserved peptide sequence, LPPVK, facilitates donation of its bound cluster to recipient proteins. Molecules containing this sequence can facilitate or slow cluster assembly, depending on their propensity to complete assembly.

Oxidoreductases

“Oxidoreductase” is the class name for enzymes that catalyze oxido-reduction reactions. Oxidoreductases catalyze transfer of electrons from one molecule to another molecule. Typically, oxidoreductases can be named oxidases or dehydrogenases. Oxidases are enzymes involved when molecular oxygen (O₂) is involved. Dehydrogenases are enzymes that oxidize a substrate by transferring hydrogen to an acceptor that is either NAD⁺/NADP⁺or a flavin enzyme. Peroxidases, hydroxylases, oxygenases, and reductases are also species of oxidoreductases. The peroxisome organelle uses peroxidases to reduce H₂O₂. Hydroxylases add —OH groups to substrates. Oxygenases add O₂ to organic substrates. Reductases catalyze reductions, acting as reverse oxidases.

Oxidoreductase enzymes are found in glycolysis, TCA cycle, oxidative phosphorylation, and in amino acid metabolism. In glycolysis, glyceraldehyde-3-phosphate dehydrogenase catalyzes reduction of NAD⁺ to NADH. Several more NADH molecules are produced in the TCA cycle after pyruvate enters the TCA cycle in the form of acetyl-CoA.

During anaerobic glycolysis, the oxidation of NADH occurs through the reduction of pyruvate to lactate as lactic acid. GAPDH acts as reversible metabolic switch under oxidative stress when antioxidants, especially NADPH, are needed to protect cells from further damage. Under oxidative stress conditions GAPDH is inactivated switching the metabolic flux from glycolysis to the pentose phosphate pathway, thereby generating increased amounts of NADPH. NADPH is then available for antioxidant-systems including glutaredoxin and thioredoxin and for the recycling of glutathione. Lactate feedback through LDH occurs when lactate production exceeds removal. Monocarboxylate transporters are responsible for physically removing lactate.

Glutamate Dehydrogenase

Glutamate dehydrogenase (GDH) is a significant link between catabolic and anabolic pathways and between nitrogen and carbon metabolism in eukaryotes. Human GLUD1 (glutamate dehydrogenase 1) and human GLUD2 (glutamate dehydrogenase 2) are controlled through ADP-ribosylation, a covalent modification carried out by the gene sirt4. Caloric restriction and low blood glucose increase glutamate dehydrogenase activity to increase the amount of α-ketoglutarate. Guanosine triphosphate (GTP), palmitoyl-CoA and Zn²⁺ are inhibitory while adenosine diphosphate (ADP), guanosine diphosphate (GDP), leucine, isoleucine and valine are stimulatory.

GDH is located in mitochondria as an important branch-point enzyme carbon and nitrogen metabolism. GDH catalyzes a reversible NAD(P)⁺-linked oxidative deamidation of glutamate into α-ketoglutarate and ammonium in two reactions. The first forms a Schiff base intermediate between ammonia and α-ketoglutarate. This Schiff base intermediate is crucial because it establishes the α-carbon atom in glutamate's stereochemistry! The second involves protonating the Schiff base intermediate by transfer of a hydride ion (H⁻) from NADPH resulting in L-glutamate. GDH is exceptional because it reacts using both NAD⁺ and NADP⁺. NADP⁺ is a reactant in the reaction of α-ketoglutarate and free ammonium (NH₄⁺) to form glutamate via a hydride transfer from NADPH to glutamate. NAD⁺ is utilized in the reverse reaction, where glutamate converts to α-ketoglutarate and free ammonia via an oxidative deamidation reaction. Extensive production of ammonia by glutamate dehydrogenase is not found because of the highly toxic effects of free ammonia in cells. The ammonia produced in the reverse reaction of GDH is converted to urea before being excreted as NH4⁺ in the urine.

The Gibbs free energy change for the conversion of glutamate to α-ketoglutarate is 3.7 kcal/mol. The reaction may be necessary to maintain re-dox equilibrium to re-oxidize the excess of NADH produced during glycolysis.

GDH is down-regulated by the cell's high energy state and up-regulated when ADP is increased. During the formation of α-ketoglutarate GDP and ADP positively regulate GDH in mammals, and GTP, ATP, leucine, and coenzyme inhibit the enzyme. At low energy levels ammonium is formed and secreted from the cells.

Alanine Transaminase—ALT

Alanine transaminase (ALT) catalyzes transfer of an amino group from alanine to α-ketoglutarate, in a reversible transamination reaction yielding pyruvate and glutamate.

L-glutamate+pyruvate [symbol] α-ketoglutarate+L-alanine

ALT is a cytoplasmic, i.e., extramitochondrial, enzyme that participates in cellular nitrogen metabolism and also in liver gluconeogenesis starting with precursors transported from skeletal muscles.

Aspartate Transaminase—AST

Aspartate transaminase (AST) catalyzes the reversible transfer of an α-amino group between aspartate and glutamate. AST catalyzes the interconversion of aspartate and α-ketoglutarate to oxaloacetate and glutamate within the mitochondrial matrix. AST is instrumental for metabolite exchange between cytosol and mitochondrion.

Aspartate+α-ketoglutarate⇄oxaloacetate+glutamate

AST is significant for amino acid metabolism and provides a major route for importation of reducing equivalents into mitochondria through participation in the malate:aspartate shuttle. AST is identical to plasma membrane fatty acid binding protein, a transporter of long-chain free fatty acids (FFA) through the plasma membrane. The transport of FFAs is upregulated in response to ethanol exposure. Longer chains and higher melting point lipids such as cholesterol may be defenses the cell has at its disposal to overcome the fluidity increase caused by ethanol and similarly acting compounds.

FFAs are breakdown products of triglycerides generally recognized as uncouplers of oxidative phosphorylation. The fatty acid molecule loses its negative charge when it binds H⁺. The neutral long chain carbon molecule then is lipid soluble and is able to cross the membrane using the bound proton as a carrier. The flux equilibrium will be in the direction of higher H⁺ concentration to lower and will therefore tend to reduce the IMM proton gradient and membrane electrical potential. One might expect that FFAs reduce the transmembrane potential because the higher the H⁺ concentration, the greater percentage of FA⁻ will bind H⁺ at equilibrium. There will be more neutral FA (H⁺ bound FA) on the side that has more H⁺ available to bind. Simple kinetics would predict that when more molecules are available for contact, there will be a higher frequency of neutral FA contacting and crossing the membrane. The rate will eventually reach a steady state with the product [FA⁻]×[H⁺] reaching equilibrium. However, when the FA anion is recirculated, for example, by adenine nucleotide translocase (ANT), inter alia, which translocate FA⁻ without benefit of H⁺, FA is continuously available for H⁺ transport and thus will reduce membrane potential and its availability for ATP synthesis. The reduced efficiency results in excess heat and additional ROS. Other acidic moieties will have similar decoupling effect when the H⁺ form is a neutral molecule thereby easily co-transporting the weak acid with the proton. But since long chain carbon molecules are more soluble in the fatty environment of the membrane, the strongest H⁺ transporting activity was found for C12-C16 length saturated fatty acids and for the longer cis-unsaturated fatty acids, with a length about half the membrane thickness.

Since the cell is compartmentalized transport (see ubiquitination) is essential to maintaining appropriate metabolism. Proteins are targeted to the mitochondrial intermembrane space by several mechanisms. Some proteins are translocated through the Tom complex to be released into the intermembrane space. Other proteins are transferred from the Tom complex to the Tim complex. These stop-transfer sequences are then cleaved to release the proteins into the intermembrane space. Still others are imported to the matrix. Removal of the transport-necessary presequence by enzymes in the matrix then exposes a hydrophobic signal sequence, to target the protein back across the inner membrane to the intermembrane space.

Glyceraldehyde-3-Phosphate Dehydrogenase—GADPH

Glyceraldehyde-3-phosphate dehydrogenase (GADPH) is an important extramitochondrial enzyme catalyzing glycolysis and gluconeogenesis. GADPH controls reversible conversion of glyceraldehyde 3-phosphate (GAP) and inorganic phosphate into 1,3-bisphosphoglycerate (1,3-BPG). During the conversion of GAP to 1,3-BPG, NADH is produced with H⁺. GADPH requires: i) a NAD⁺ cofactor as an electron acceptor, and ii) inorganic phosphate. GADPH has two sulfate molecules per subunit emphasizing the importance of sulfur in ox/redox. The IMM is comparatively rich in proteins. Its protein content of about 4/5 exceeds that of: the nuclear membrane—about 2/3 protein, the ER—about 3/5 protein, and the outer mitochondrial membrane and the plasma membrane—about 1/2. The low lipid—high protein content may contribute the mitochondrion's temperature stability during active metabolism.

Phosphorus—Phosphate

Phosphorus is essential for all known living organisms. Phosphorus serves as a backbone for nucleic acids and is an integral cell membrane component, for example, as phospholipids. The phosphorus portion of the phospholipid allows water to orient with rows of phospholipid to form biologic membranes. Phosphorus ranks with nitrogen as the most needed inorganic foods required for life. ATP (adenosine triphosphate) serves as a constituent molecule for energy transfer reactions. Another triphosphate, GTP is a prime component in membrane receptors and signal transduction cascades with kinases phosphorylating and dephosphorylating proteins integral in activating or deactivating many enzymes. Many molecules must be phosphorylated to participate in enzymatic pathways.

Phosphate is obvious in its importance in the mitochondrion whose most notable function is phosphorylating ADP to produce ATP. Membranes are mostly lipid (fat, oil) and therefore impermeable to most polar or charged chemical substances. Phosphate (PO₄⁻³) being an electrically charged ion must be transported across lipid membranes. One such action is phosphate transduction through the inner mitochondrial membrane by the mitochondrial phosphate transporter. Mitochondrial phosphate transporters are members of the mitochondrial carrier family, each of which sports six-transmembrane-domain structures comprising three repeated segments of two transmembrane—helices separated that are connected by a hydrophilic loop. Mitochondrial phosphate transporter genes have been cloned from several species, and generally operate via Pi/H symport or Pi/OH antiport. The mitochondrial phosphate transporters catalyze exchange between the matrix and the cytosol. Phosphorus is also structurally important for building and maintains healthy bones and teeth. A large proportion (80-90%) of phosphorus is stored in the body as apatites in these structures. Phosphorus involvement in this variety of activities in the cell's metabolism, especially the molecular storage in bone material, and availability in multiple pathways make metabolic monitoring and control of phosphorus use and reactions important for maintenance of the organism's health. Because of phosphorus' and phosphate's involvement presence in copious interacting metabolic pathways a switch in any one of these pathways will elicit pervasive compensatory adaptations throughout the cell and organism. Readjusting the cell's early metabolic shifts back to supporting the whole organism rather than the individual cell or its lineage can prevent the cascading maladaptations that eventuate into serious disease states.

One such organism wide maladaptation results from elevated levels of serum insulin. PO₄ uptake by cells is increased by insulin. Depressed serum PO₄ will lower the Ca/PO₄ ion product and can interfere with bone structure as CA is released to the serum.

ATP and Mitochondria

The core molecule in the energy system of living cells is the phosphorus-containing adenosine triphosphate (ATP).

ATP is integral in most of the intracellular energy transport. Bulk energy is stored in animal cells in carbohydrates like glycogen and in various fats. When metabolism is progressing, that is the cell requires a chemical reaction for its operations, stored chemical energy must be harvested. Fuel compounds such as glucose (or other carbon source) are oxidized with transference of chemical energy to adenosine phosphate. The most common reaction in this genre is simply upgrading adenosine diphosphate (ADP) to ATP. This energy source molecule, when linked to other chemical reactions, then becomes available to metabolism for many cell functions, such as transporting components across membranes, driving additional chemical reactions, contracting muscles and producing heat. ATP is efficiently produced in the mitochondrion using oxidative phosphorylation, but alternative production pathways include anaerobic and aerobic glycolysis paths that occur in the cytoplasmic space.

The mitochondrion is a prolific heat generator, especially for warm-blooded animals. Maintenance of the H⁺ electrochemical gradient comprises exothermic biochemical reactions thereby elevating local temperatures, first in the mitochondria themselves, and then by conduction or convection through the cell and then throughout the organism using the circulatory system. Brown fat cells have differentiated to increase their reactions to pump up the gradient (and allowing leakage so the gradient does not become too strong). In cells with compromised OXPHOS metabolism, the normal heat generation will not happen in the mitochondria. Mitochondria will be relatively cool with respect to other cells. Such temperature differences can be a nano signal indicating compromised OXPHOS activity.

One example of altered phosphorus metabolism is evident in cancer cells. As a class these cells demonstrate a massive shift from oxidative phosphorylation in the mitochondrion to generally aerobic glycolysis in the cytoplasm for production of ATP. This adaptation appears fundamental for support of a cancer cell's metabolic needs.

Glucose is considered a model carbon fuel source in the cell. The liver makes glucose available to other body tissues and hormones, most significantly glucagon and insulin, control circulating levels. Initiation of glucose metabolism occurs in the cytosol. Here a glucose molecule is converted to 2 pyruvate molecules. Pyruvate then moves to mitochondria for further oxidation eventually to CO₂. This oxidative phosphorylation (OXPHOS) process under normal circumstance produces most of usable energy as ATP that we obtain from metabolizing glucose. OXPHOS starts with oxidation of pyruvate to acetyl CoA which the citric acid cycle converts to ATP and CO₂. As an alternative to glucose fatty acids can be oxidized to make acetyl CoA, which then enters the citric acid cycle. Thus, the activities carried out by transporters and enzymes of mitochondria and the citric acid cycle are of particular concern for supplying ATP to cells from consumption of sugars as glucose of fatty acids. During this process, NAD⁺ is reduced to make and players in the oxidative breakdown of both carbohydrates and fatty acids. The oxidation of NADH and FAD is reduced to make FADH₂ used to drive other metabolic reactions most significantly to produce a proton (or H⁺) gradient across the inner mitochondrial membrane. Maintenance and restoration of this gradient is essential for the mitochondrion's production of ATP.

Generation and storage of metabolic energy are required activities for all cells, and two cytoplasmic organelles are specifically devoted to energy metabolism and the production of ATP. Mitochondria are responsible for generating most of the useful energy derived from the breakdown of lipids and carbohydrates, and chloroplasts use energy captured from sunlight to generate both ATP and the reducing power needed to synthesize carbohydrates from CO₂ and H₂O. Chloroplasts, present only in plants, have relevant similarities to the older mitochondrion organelle found in both plants and animals.

Rather than being synthesized on membrane-bound ribosomes and translocated into the endoplasmic reticulum, proteins destined for mitochondria, chloroplasts and peroxisomes are synthesized on free ribosomes in the cytosol and imported into their target organelles as completed polypeptide chains. Mitochondria and chloroplasts also contain their own genomes, which include some genes that are transcribed and translated within the organelle. Protein sorting to these cytoplasmic organelles is a complex process involving carriers, repeated phosphorylations and dephosphorylations and energy to support these processes. The ultimate energy source within the mitochondrion is the proton (H⁺) gradient across the inner mitochondrial membrane. The separation of the H⁺ ions by the membrane allow countertranslocation of H⁺ and other molecules to be energetically favorable. The most discussed of these exchanges involves H⁺ transport into the matrix and ATP production.

Mitochondria

Mitochondria are the major players in generation of metabolic energy in eukaryotic cells. They harvest energy derived from the breakdown of carbohydrates and fatty acids to make ATP by OXPHOS. Most mitochondrial proteins are translated on free cytosolic ribosomes and imported into the organelle by specific targeting signals. Mitochondrial DNA encodes tRNAs, rRNAs, and some mitochondrial proteins, but the large majority of mitochondrial proteins are encoded by nuclear DNA and produced in extramitochondrial space. Mitochondria have only a few or their mitochondrial membrane proteins encoded by their own genomes and translated within the organelle; the predominance of proteins is encoded by the nuclear genome and imported from the cytosol. Mitochondria are enclosed by a double-membrane system, an inner (IMM) and an outer (OMM) membrane. The matrix is the inside structure of the mitochondrion with many folds that increase IMM surface area. This matrix portion comprises the most active portions of the mitochondrion. The matrix contains the mitochondrial genetic material and predominant active proteins for OXPHOS.

The mitochondrial proteins made in the cytoplasmic space are targeted to mitochondria with an amino-terminal presequence of positively charged amino acids. Proteins are maintained in a partially unfolded pseudo-linear arrangement by cytosolic Hsp70 that is recognized by a receptor on the surface of mitochondria. The unfolded polypeptide chains are then translocated through the Tom complex in the OMM and transferred to the Tim complex in the inner membrane. The transmembrane charge component of the electrochemical gradient is required for movement across the inner membrane. Once inside the presequence is cleaved by a matrix protease, and then a mitochondrial Hsp70 binds the polypeptide chain to cross the IMM. A mitochondrial Hsp60 then folds the imported polypeptides within the matrix. Mitochondrial membrane activities regulate transport of mitochondrial GSH (mGSH). The physical properties are regulated by fatty acid composition in the mitochondrial membrane and especially by the cholesterol/phospholipid molar ratio. For example, cholesterol enrichment causes mGSH depletion which can tilt metabolism of a cell towards cell death. Mitochondria incorporate less cholesterol in their membranes than plasma membrane. Cholesterol loading in mitochondrial membranes results in reduced activity of several membrane carriers, e.g., GSH transport system, with no apparent effect on other transporters. The effect of cholesterol on ROS mitigation processes in thus especially pronounced.

Cholesterol impairs transport of mGSH increasing susceptibility to oxidative stress and cell death. Cholesterol, especially in mitochondria, may be an important target for controlling mitochondrial damage and therethrough modulating metabolic health.

Mitochondrial cholesterol transport is preferentially regulated by the steroidogenic acute regulatory domain 1 (StARD1), and other members of a family of lipid transporting proteins that contain StAR-related lipid transfer (START). StARD1 is an OMM protein that is instrumental in cholesterol transfer to the IMM for metabolism by cholesterol side chain cleavage enzyme (CYP11A1) as it generates pregnenolone, the precursor of steroids. Pregnenolone synthesis in mitochondria is cholesterol limited. Caveolin-1 (CAV1), a key component of caveolae, is important for guiding mitochondrial cholesterol. CAVs bind cholesterol with high affinity. CAVs move between cell compartments, e.g., mitochondria, ER and plasma membrane to regulate movement of cholesterol and eventual distributions within cells. Inactivating CAV1 increases mitochondrial cholesterol leading to mGSH depletion and increased ROS damage that can participate in apoptotic control. Most proteins are targeted to mitochondria by amino-terminal sequences of 20 to 35 amino acids (called presequences) that are removed by proteolytic cleavage following their import. The transfer of high-energy electrons from NADH and FADH₂ to O₂ is coupled to the transfer of protons from the mitochondrial matrix to the intermembrane space. Since H⁺ are charged particles, this transfer establishes an electric potential across the IMM, with the matrix being negative. During protein import, this electric potential drives translocation of the positively charged amino acid presequence. Since these interactions of polypeptide chains with molecular chaperones depend on ATP, protein import requires ATP both outside and inside mitochondria to augment the transmembrane electromotive force.

Mitochondrial membrane proteins contain hydrophobic stop-transfer sequences that halt their translocation through the Tom or Tim complexes and lead to incorporation into the outer or inner membranes, respectively. A healthy IMM is essential for regular ATP generation. The membrane is protein-rich comprising a protein component in excess of 2/3. Its surface area is magnified by the multiple folds producing its cristae. This permits the proton gradient to have a larger area to act through the proteins that transport and react OXPHOS metabolism. Since protons are the smallest of ions, the proteins and lipids of the inner membrane must be especially non-leaky with respect to atoms and molecules, especially charged substances. The mitochondrion is thus a critical component of the cell's metabolic process. The mitochondrion is distinguished in that it is the only organelle (except for chloroplasts in photosynthesizing organisms) with genomic material outside the cell nucleus. Vitamin K2 is an important electron carrier in mitochondrial membranes (similar to its actions in bacteria).

Mitochondrial DNA (mtDNA) is a double stranded circular genome very similar in structure to a bacterial genome. One major difference is that the mitochondrial genome does not contain genes sufficient for most mitochondrial functions or even to support mitochondrial survival. More than 1500 different proteins are found in the mitochondrial proteome; but only 14 proteins are coded in its mtDNA:—humanin, a protein that leaves the mitochondrion and exerts anti-apoptotic activity in the cytosolic space; 2 of the 13 component proteins of ATP synthase (proton port); 3 of the 19 cytochrome c oxidase protein components; 1 of the 11 protein components of cytochrome b; and 7 of the 44 complex 1/NADH: ubiquinone oxidoreductase protein components. Consequently, interfering with transcription, translation, RNA processing, polypeptide assembly from nuclear or mtDNA can profoundly affect mitochondrial functions and may change how the cell uses oxygen, glucoses, fatty acids, etc., and how cell building blocks and the ATP energy source are created and maintained. Since the mitochondrion is dependent on the nDNA and protein products thereof for maintaining the mt-proteome and proteins resulting from nuclear transcription and cytoplasmic translation the ability of the mitochondria to signal the nucleus when mitochondria need to build or replace their proteome is paramount. Mitochondrial-nuclear proximity is beneficial in this regard. Proximity can be supported by mitochondrial-endoplasmic reticulum (ER) interfaces with the ER membrane continuous with the nuclear membrane to provide an anchor. Cytoskeletal activity then tethers and positions mitochondria at their needed sites. Similarly, the ER, nucleus and other cell components needing energy must signal their energy needs to mitochondria so that sufficient ATP is available. G-Protein Pathway Suppressor 2 is a nuclear encoded protein that becomes bound to mitochondria, but is released at times of oxidative stress to stimulate mt-protein production.

Transcription signals from other organelles also induce mt-protein synthesis, some of which comprise enzymes for ATP production and transport of the necessary biomolecules. Others comprise proteins that modify, e.g., phosphorylate or dephosphorylate, mitochondrial pathways, improve substrate delivery, bind to specific proteins to increase or decrease that specific protein's actions. Mitochondria are transported along the cells' microtubules using, for example, the kinesin-1 motor (Kif5b, KHC).

Classically, i.e., under normal circumstances, most of the available cellular energy is produced in mitochondria using oxidative phosphorylation (OXPHOS). OXPHOS is a very efficient pathway that couples electron transport and resultant proton (H⁺) gradient across the mitochondrial inner membrane to convert ADP and phosphate to ATP. Although ideally all the oxygen should be reduced to H₂O by a four-electron reduction reaction catalyzed by cytochrome oxidase, even under normal conditions, a small fraction of O₂ is only reduced by one, two, or three electrons, yielding superoxide anion (O₂⁻), hydrogen peroxide (H₂O₂), and the hydroxyl radical (·OH), respectively. (“Radical” or “free radical” are terms describing molecules that are unstable because they carry an unpaired electron.)

Mitochondrial pathways are also involved in other important cellular functions including, but not limited to: Ca²⁺ homeostasis, heme biosynthesis, nutrient metabolism, steroid hormone biosynthesis, ammonia clearance, initiating and/or supporting metabolic and signaling pathways leading to apoptotic cell death and to autophagy. Mitochondria, as organelle inclusions in the surrounding cell, and the surrounding cell continuously interact through energy production and supply of gene products (mitochondrial proteins), transporting and using or eliminating other materials—such as amino acids and nitrogen compounds, oxidized and reduced substrates, cofactors, H⁺, ion gradients, etc. to support demands of mitochondrial metabolism, cellular metabolism and metabolism of the tissue and organism. MtDNA also provides coding for mitochondrial RNA and the tRNAs used for polypeptide synthesis in the mitochondrion. Transport from and to the mitochondrial matrix requires specialized transport structures (mostly encoded by nDNA) and cellular transport to get to the OMM. These co-dependencies mean that any mutation or modification (think epigenesis) involving nuclear or mtDNA can be observed in overall cell function and in mitochondrial supporting functions. As a corollary, a mutated mtDNA often induces compensatory or corrective activities in cytosolic space and changes in nuclear DNA expression often induce profound effects in the cell's mitochondria, including major effect on OXPHOS.

Oxidative phosphorylation comprises a series of ordered steps, applied to pyruvate and resultant intermediate products, through and across multiple redox centers organized in five protein complexes in the IMM. The transfer of electrons produces a H⁺ gradient across the IMM to drive ATP production.

The cell has alternative means for producing ATP. Cytoplasmic mediated anaerobic and aerobic glycolysis can consume glucose and produce lactic acid or alternatives such as the amino acid, alanine. Products of non-OXPHOS metabolism can be used for synthesis reactions in the cell. And synthesized alanine can be released as a carrier of nitrogen thereby ridding the cell of ammonia.

In the presence of oxygen and lactic acid (produced in shunting pyruvate from OXPHOS) the alternatives to OXPHOS result in increased cell mass and additional nucleic acid synthesis. These processes support cell proliferation/division. Accelerated cell division can in itself confer a selective advantage over a population of normally dividing cells. Thus, early intervention to control or minimize pyruvate diversion from OXPHOS can be an effective brake on proliferation of these more rapidly dividing cells and may arrest progression to a cancerous disease state.

A concomitant effect to lactate production is a decreased cytosolic pH due to the additional H⁺ from lactic acid ionizing to lactate and H⁺. The decreased pH takes many enzymes out of optimal ranges for catalyzing reactions. A few enzymes, including, but not limited to: PGK and PGAM are not compromised by the lactic acid induced decreased pH and at least GAPDH becomes more active with lowered pH. Protons, the driving force of ATP production in the mitochondrion, when in the cytosol, can inhibit several glycolytic enzymes and favor alternative metabolic pathways for glucose/pyruvate metabolism, e.g., pyruvate carboxylation. Another effect of lactic acid acidification is a decrease in 2-deoxyglucose transport into the cell (a measure of glucose uptake). An expected result of decreased glucose uptake would be for the cell to increase expression of the glucose transport protein GLUT1 to maintain cytosolic glucose concentration. But consistent with the lack of GLUT1 synthesis, glucose concentration in the cytosol actually increases in these acidic conditions. Obviously the OXPHOS path is not consuming glucose product and has been shifted to support other metabolic pathways.

GLUT1 is also the carrier bringing dehydroacscorbic acid (oxidized vitamin C) into mitochondria where it is restored to the antioxidant, ascorbic acid. Vitamin C is important for scavenging mtROS and protecting mitochondrial genomes. However, when the proton gradient and membrane potential across the IMM diminish, dehydroascorbic acid uptake and antioxidant protection are severely compromised. Protecting the IMM and supplementing sufficient vitamin C are important factors supporting a healthful metabolism. Ascorbate is involved at least in the biotin, cobalamin, folate, lipoic acid, niacin, pyridine synthetic, ubiquinone, vitamin B6, vitamin D, vitamin E, vitamin K, thiamine, riboflavin, retinoid, pantothenic and NAD metabolic pathways. Maintenance and support of one or more of these may be featured in rebalancing metabolism.

A transporter that carries glucose and galactose is sometimes referred to as the sodium-dependent hexose transporter, known more formally as SGLUT-1. In accordance with its name this receptor/transporter molecule transports both glucose (or galactose) and Na⁺ ion into the cell and can not transport either alone. The process of transport by SGLUT-1 involves a series of conformational changes induced by binding and release of sodium and glucose, following this general process: i) SGLUT-1 is initially oriented facing extracellularly where it can bind sodium, but not glucose; ii) sodium binds to induce a conformational change that opens the glucose-binding pocket; iii) glucose binds and the transporter reorients in the membrane to bring the sodium and glucose binding sites to the cytoplasmic side; iv) sodium dissociates into the cytoplasm which destabilizes glucose binding; v) glucose dissociates into the cytoplasm; and vi) the unloaded transporter reorients back to its original, outward-facing position. Other sugars use other transport pathways. For example, fructose is not co-transported with sodium. Rather it can be incorporated into a cell using another hexose transporter (GLUT5). The inhibited OXPHOS pathway may have another selective advantage. In the absence of lactic acidification of the cell glucose is rapidly consumed which may initiate cell death when glucose availability is diminished. By inhibiting the OXPHOS mediated glycolysis, glucose is preserved and this cell has a survival advantage under these conditions.

A two part survival advantage of these OXPHOS reduced cells may result from their hyper consumption of glucose, thereby starving neighboring cells of this resource. And then when glucose supply is short, the prescient activation of lactate generation mechanisms confers survivability to the very cells that had depleted glucose concentrations. As an alternative to using glucose to generate highly energetic ATP, the mitochondrion can rebalance ATP production responsibilities to the cytoplasmic space and divert glucose consumption to a) synthesize reducing equivalents e.g., NADPH useful for making fatty acids, b) provide ribose-5-phosphate for nucleic acid generation, and/or c) make erythrose-4-phosphate for aromatic amino acid generation. This pathway is separate from the heme synthesis path where G6P can serve as a source of glycine before export to cytoplasmic space where it is processed before reentering the mitochondrion as coproporphyrinogen III where the mitochondrion completes the heme synthesis process. Glucose availability may be modulated, for example, with one or more of the following compounds: dapagliflozin, empagliflozin, canagliflozin, ipragliflozin (ASP-1941), tofogliflozin, sergliflozin etabonate, remogliflozin etabonate (BHV091009), ertugliflozin (PF-04971729/MK-8835), sotagliflozin, and other compounds of the gliflozin class. Early in the ribose pathway glucose-6-phosphate dehydrogenase converts the G6P to 6-phosphoglucono-δ-lactone with NADPH as a byproduct. The 6-phosphoglucono-δ-lactone is converted by 6-phosphogluconolactonase to 6-phosphogluconate which when acted on by 6-phosphogluconate dehydrogenase produces another NADPH and forms ribulose-5-phosphate.

Then ribulose 5-phosphate isomerase makes ribose 5-phosphate that is acted upon by ribulose 5-phosphate 3-epimerase to form xylulose-5-phosphate.

Then a xylulose 5-phosphate and a ribose 5-phosphate are transformed to glyceraldehyde 3-phosphate and sedoheptulose 7-phosphate by transketolase. Transaldolase reacts these to make erythrose 4-phosphate and fructose 6-phosphate which are converted to glyceraldehyde 3-phosphate and fructose 6-phosphate by transketolase.

Alternate paths to ETC production of ATP are possible when the cytosol rebalances to make more ATP and the co-generated lactic acid. The lactic acid production has a cost in less ATP energy being available from mitochondria, but allows the mitochondria to switch pathways for synthesis of other cellular components that may face extreme demands as a cell's growth and division rates may be increasing.

Calcium

Calcium (Ca) is most frequently bound with phosphate in the hydroxyapatite structure of bone (Ca₁₀(PO₄)₆(OH)₂). Thus, bone provides a bank for both Ca⁺⁺ and PO₄⁻³ that is recruitable by hormones and affected by nutrition and vitamin levels, e.g., vitamin D. Outside bone and teeth Ca is involved as a cofactor in many reactions. For example, Ca flux is necessary for muscle contraction, including cardiac muscle contraction. In this muscle contraction example ATP is necessary to break the actin/myosin bonding to allow muscle tissue to relax in preparation for its next contraction. Many cells have receptors that signal intracellular action by increasing Ca flux into the cell. Other cells neighboring or distant are affected when Ca activated secretory cells release local or systemic hormones. Ca movement into the cell is a common activation feature. Intracellular free Ca concentration is maintained low by active transport that is powered by the ATP the cell produces. Organelles, especially mitochondria and endoplasmic reticulum also participate in maintaining a low cytosolic free Ca concentration. Release of Ca by these organelles is one mechanism through which apoptosis is initiated to destroy cells whose metabolism has deviated from normal organism maintenance requirements. In some cells vitamin D receptors act as transcription factors initiating pathways leading to differentiation/proliferation.

Vitamin K is a name for a group of structurally similar, fat-soluble vitamins the human body requires for controlling binding of calcium in bones and other tissues. A vitamin K-related modification of proteins allows Ca binding. Without vitamin K, blood coagulation is seriously impaired, and uncontrolled bleeding occurs. Chemically, the vitamin K family comprises 2-methyl-1,4-naphthoquinone (3-) derivatives. “Vitamin K” includes two natural vitamers: vitamin K1 and vitamin K2. Vitamin K2, in turn, consists of a number of related chemical subtypes, with differing lengths of carbon side chains made of isoprenoid groups of atoms. Vitamin K is a coenzyme for vitamin K-dependent carboxylase, an enzyme required inter alia to synthesize proteins involved in blood clotting and bone metabolism. Prothrombin (clotting factor II) is a vitamin K-dependent protein in plasma.

Mitochondria require electron flux across the IMM to make ATP for cellular energy metabolism. This process uses the ETC a collection of protein complexes populating the IMM. ETC defects can promote development of neurodegenerative diseases. For example, mutations in the gene encoding PTEN-induce putative kinase 1 (Pink1), a protein that signals mitochondrial dysfunction, cause familial forms of Parkinson's disease.

A proton gradient-dependent calcium pump pumps Ca from the cytosol to mitochondrion when cytoplasmic calcium concentrations increase, possibly in response to stimulus from a plasma membrane receptor allowing interstitial Ca to enter or perhaps to restore Ca back to mitochondria after release to effect cytosolic metabolic activity or temper mitochondrial TCA cycle activity. Mitochondrial calcium stimulates pyruvate dehydrogenase, isocitrate dehydrogenase and α-ketoglutarate dehydrogenase which increases the cycling rate of the TCA cycle. Cytoplasmic Ca is important as an intracellular signal in many cells. For example, in muscle, increased cytoplasmic calcium concentration initiates Ca binding to myosin which allows actin to bind, and then, in an ATP dependent reaction Ca is released either to be recycled for contraction or returned to the sarcoplasmic reticulum.

Pyruvate dehydrogenase activity can be turned off by pyruvate dehydrogenase kinase (PDK) which stops conversion to acetyl-CoA and prevents it from ATP production through OXPHOS. Dichloroacetic acid inhibits PDK and thus can help rebalance metabolism from lactic acid generation from pyruvate towards OXPHOS metabolism.

Ca is instrumental in delivering intracellular switching signals. One pathway controlled by Ca features phospholipids such as phosphatidylinositol 4,5-bisphosphate (PIP₂) and its derivatives. Several hormones and growth factors including, but not limited to: 5-HT2 serotonergic receptors, al adrenergic receptors, calcitonin receptors, H₁ histamine receptors, metabotropic glutamate receptors—Group I, M₁, M₃, and M₅ muscarinic receptors, thyroid-releasing hormone receptor, platelet derived growth factor, fibroblast growth factor, cannabinoid receptors, etc. are available to stimulate hydrolysis of PIP₂ by phospholipase C to form diacylglycerol (DAG) and inositol 1,4,5-trisphosphate (IP₃). DAG remains incorporated in the membrane while IP₃ is liberated into the cytoplasm. Examples of substances that can inhibit PIP₂ and related pathway activity, directly or indirectly, include but are not limited to: aminosteroid, edelfosine, prozosin, propranolol, o-phenanthroline, adrenergic inhibitors including both a and 3 blockers, trazodone, mirtazapine, ergot alkaloids including metergoline, ketanserin, ritanserin, nefazodone, clozapine, olanzapine, quetiapine, risperidone, asenapine MDL-100,907, cyproheptadine, pizotifen, LY-367,265, AMDA and derivatives, hydroxyzine, 5-MeO-NBpBr, niaprazine, AC-90179, nelotanserin (APD-125) eplivanserin, pimavanserin (ACP-103), 2-alkyl-4-aryl-tetrahydro-pyrimido-azepines, volinanserin, thioperamide, JNJ 7777120, atropine, hyoscyamine, scopolamine, diphenhydramine, dimenhydrinate, dicycloverine, thorazine, tolterodine, oxybutynin, ipratropium, mamba toxin MT7, mamba toxin MT1, mamba toxin MT2, pirenzepine, telenzepine, chlorpromazine, haloperidol, rimonabant, cannabidiol, Δ⁹-tetrahydrocannabivarin, ALW-II-41-27BGJ398, FGF401, SSR128129E, SU 54, afatinib, axitinib, cacozatinib, ceritinib, crizotinib, eriotinib, gefitinib, lapatinib, ponatinib, NVP-BHG712, regrorafenib, sunitinib, vandetanib, JI-101, etc. DAG stimulates protein kinase C and sequelae while IP₃ causes release of Ca in the cell. For example, in response to growth factor binding and activating its receptor, the receptor protein-tyrosine kinases is activated so it can bind phospholipase C-γ (PLC-γ) which it phosphorylates to promote its catalytic activity that cleaves PIP2. Staurosporine is an inhibitor of protein kinase c.

DAG derived from PIP2 stimulates protein-serine/threonine kinases which often act as important controllers of cell growth and subsequent differentiation. Protein kinase C is one example of an intracellular signal that when dispatched supports superfluous growth and tumor development. Phorbol esters have been recognized as a causative factor in tumor initiation and growth. Phorbol esters act as an analogue of DAG to stimulate protein kinase C which is free to activate other intracellular targets, including the MAP kinase pathway. The result of protein kinase C activation is transcription factor phosphorylation to alter gene expression so that it stimulates proliferation of the affected cell(s).

IP₃ binds to ER receptors associated with Ca transmembrane channels. This allows passage of Ca from the ER into the cytoplasm where it affects activity of several target proteins, e.g., protein kinases and phosphatases. Members of the CaM kinase family are one target of Ca-calmodulin. These phosphorylate several different proteins, including, but not limited to: metabolic enzymes, ion channels, transcription factors, etc. Different isoforms of CaM kinase are active in different tissues. CaM kinases can regulate gene expression by phosphorylating transcription factors. One transcription factor phosphorylated by CaM kinase is CRE-binding protein (CREB), which interacts with the cAMP signally pathways which also intersect through adenylyl cyclases and phosphodiesterases activated by Ca/calmodulin complex. Co-regulation of Ca channels by cAMP, and the phosphorylation of several target proteins by both protein kinase A and Ca/calmodulin-dependent protein kinases. The cAMP and Ca signaling pathways interconnect to regulate many cellular responses.

Ca is an important second messenger released in response to a primary stimulus. IP₃-mediated release of Ca from ER is only one mechanism that causes increased intracellular Ca concentrations. Entry of extracellular Ca through Ca channels in the plasma membrane can also increase cytosolic Ca concentration. Often transient IP₃ induced increases in intracellular Ca are followed with a sustained increase from extracellular Ca entry. This release of stored Ca leads to large increases in cytosolic Ca to carry out cell functions. Thus, Ca must be considered as a versatile second messenger for controlling a wide range of cellular processes.

PIP₂ is not only a source of DAG and IP3. PIP₂ also initiates a distinct second messenger pathway with a role in regulating cell survival. In this pathway, when phosphatidylinositide (PI) 3-kinase phosphorylates PIP2 on the 3 position of inositol phosphorylation of PIP₂ yields phosphatidylinositol 3,4,5-trisphosphate (PIP₃), another intracellular signal. Protein-serine/threonine kinase (aka Akt) is an important target of PIP₃, a supporter of cell survival that upon activation phosphorylates several target proteins, including proteins that directly regulate cell survival, transcription factors and other protein kinases that regulate cell metabolism, and transcription factors and other protein kinases that regulate protein synthesis.

The MAP kinase pathway is an alternative substrate pathway for protein kinase C. MAP kinase pathway includes a cascade of protein kinases that have been highly conserved in the development processes. This pathway plays central roles in signal transduction in virtually all eukaryotic cells. The core of the MAP kinase pathway is a family of protein-serine/threonine kinases named mitogen-activated protein kinases (MAP). These are activated by a variety of growth factors or other signaling molecules. Throughout the animal kingdom MAP kinases are ubiquitous regulators of cell growth and differentiation. PD098059, UO126 and SB203580 are examples of compounds that exert inhibitory effect against MAP kinases.

As an example, extracellular signal-regulated kinase family (ERK) coordinates cell proliferation when induced by growth factors that activate protein-tyrosine kinase receptors or G protein-coupled receptors. Protein kinase C is another ERK pathway activator. Cell Ca is an important regulator of ERK enzymes. Imatinib, gefitinib, erlotinib, sunitinib, and cabozantinib are examples of compounds that exert inhibitory effect countering tyrosine protein kinase or other ERK activation either directly of via growth factor inhibition.

Proliferation involves the procession through mitosis. Proliferation of a cell, growth and then division to form to cells, is normally tightly controlled within the organism. However, when metabolism goes awry, the cell responds by activating a set of genes corresponding to the altered metabolic activities or needs. Occasionally the activated genes set in motion cellular activities leading to accelerated cell division. The accelerated cell division sometimes evades brakes normally imposed by the organism's metabolic controls. ERK pathways are sometimes involved in accelerated and/or uncontrolled proliferation. The cell cycle of proliferation consists of a state of quiescence (G₀), a first gap phase (G₁), the DNA synthesis (S phase) a second gap phase (G₂), then mitosis (M), the actual cell division phase. Retinoblastoma protein (Rb) phosphorylation by a CDK/cyclin complex allows release of transcription factor E2F that can activate several genes including, but not limited to: cyclins A, D and E. CIP/KIP family members p21CIP1, p27KIP1 and p57KIP2 assist CDK/cyclin association. p53 regulates p21CIP1. p16INK4a and p14ARF are tumor suppressors (encoded by the same gene in overlapping reading frames)!!-p16INK4a is inactivated in many cancers. p14ARF can maintain cycle arrest in G₁ or G₂. It complexes with MDM2 to prevent MDM2 from neutralizing p53 thereby transcriptionally activating cyclin-dependent kinase inhibitor 1A or inducing apoptosis. Hyperexpression of cyclins is one hallmark of cells tending to hyperproliferate. The ERK pathway controls, for example, c-jun activity. Constitutively active ERK increases c-jun transcription and stability through CREB and GSK3. C-jun is thus activated with its downstream targets including, but not limited to: RACK1 and cyclin D1, etc. RACK1 enhances JNK activity, with activated JNK signaling subsequently phosphorylating and upregulating c-jun activity.

Phosphorylation of Jun at serines 63 and 73 and at threonines 91 and 93 leads to increased transcription of c-jun target genes. Regulation of c-jun activity can thus be achieved by N-terminal phosphorylation by the Jun N-terminal kinases (JNKs). Jun's activity (AP-1 activity) in stress-induced apoptosis and cellular proliferation is regulated by its N-terminal phosphorylation. Loss of proliferative control leading to oncogenic transformation by ras and fos requires Jun N-terminal phosphorylation at Serine 63 and 73.

C-jun is required for progression through the G₁ phase of the cell cycle. C-jun regulates the transcriptional level of cyclin D1, a major retinoblastoma kinase. Rb is a growth suppressor that is inactivated when phosphorylated. When c-jun activity is absent or blocked, expression of p53 (cell cycle arrest inducer) and p21 (CDK inhibitor and p53 target gene) increase. This can slow or arrest the cell cycle preventing continued hyperproliferation. On the other hand, hyperexpression of c-jun to increase activity, results in decreased levels of p53 and p21, with resultant accelerated cell proliferation.

Interfering with N-terminal phosphorylation of c-jun can slow cell cycle progression and help rebalance activities of the cells. SP600125 and AS601245 are two examples of compounds that effectively inhibit or prevent c-jun N-terminal phosphorylation. Inhibiting other proteins supporting cell cycle progression can have similar effect or may be used to augment or synergize other cell cycle modulations.

Ras, a GTP-binding protein, activates two protein kinases upstream of ERK. Ras activates Raf protein-serine/threonine kinase. Raf in turn phosphorylates MAP kinase/ERK kinase (MEK) which then activates members of the ERK family by phosphorylation of both threonine and tyrosine residues separated by one amino acid (e.g., threonine-183 and tyrosine-185 of ERK2). ERK then phosphorylates several targets, including e.g., other protein kinases and transcription factors.

Ras proteins were first identified as the oncogenic proteins. Inhibiting Ras function, e.g., by expressing a dominant negative Ras mutant can stop growth factor-induced cell proliferation. Ras is a viable target for impeding abnormal growth characteristics.

Ras proteins are guanine nucleotide-binding proteins that function by alternating between inactive GDP-bound and active GTP-bound forms. Ras is activated by guanine nucleotide exchange factors that stimulate release of bound GDP in exchange for GTP. Ras-is turned off by GTP hydrolysis controlled by interaction of Ras-GTP with GTPase-activating proteins. In human cancers GTP hydrolysis by the Ras proteins is inhibited. The Ras proteins are similar to a large family of approximately 50 related proteins, often called small GTP-binding proteins. These analogous sub-families direct other a variety of cellular activities. E.g., Rab proteins regulate vesicle trafficking; Ran proteins direct nuclear protein import; and Rho helps organize the cytoskeleton. These activations are often phosphorylation reactions that require both Ca and Mg.

When Raf initiates a protein kinase cascade leading to ERK activation ERK phosphorylates several target proteins, including protein kinases. Some activated ERK enters the nucleus to phosphorylate and direct activities of target transcription factors.

A primary response to growth factor stimulation is rapid transcriptional induction of a family of serum response element (SRE) containing genes called immediate-early genes. SRE is recognized by a complex of transcription factors including the serum response factor (SRF) and Elk-1. ERK phosphorylates and activates Elk-1, thereby linking the ERK family of MAP kinases and immediate-early gene induction. The interconnections enable controlling several proliferation-favoring metabolic pathways with one or a small number of chemical or biologic interventions.

Second messengers are also derived from other phospholipids. Several growth factors stimulate phosphatidylcholine which then provides an alternate source of DAG. PIP₂ hydrolysis is a transient response to growth factor stimulation. In contrast, hydrolyzed phosphatidylcholine is stable for several hours, thereby providing a sustained source of diacylglycerol important in signaling long-term responses, such as cell proliferation.

Magnesium

Magnesium (Mg) is a divalent cation similar in this respect to Ca. Mg content is much lower than Ca and so poorly competes with Ca on divalent cation transporters. But Mg has immense importance due to its unique characteristics in supporting phosphoryl transfer underlying its participation as a cofactor for in excess of 300 human enzymes, especially important in this discussion for supporting phosphate related reactions including nucleic acid synthesis and repair, ATP production and enzymatic protection from oxidative stress. For example, in nucleotide excision repair (correcting this type of DNA copy mistake), Mg coordinates activity of over 20 enzymes. Magnesium binding is especially sensitive to H⁺ concentration (pH) losing affinity for phosphates such as ATP as the pH falls. Amines, usually polyamines, which increase charge as pH decreases can competitively displace Mg as the charge increases. Displacement of Mg is a paramount concern because most of the cell's Mg is bound to nucleic acid polyanions. Mg serves as a counterion protecting access to purine and pyrimidine bases. Mg through its propensity to bind phosphate is a frequent cofactor for ATPases. Mg also protects nuclear DNA by binding and stabilizing histones.

In protein synthesis, Mg binds rRNA coordinating its 3-D structure. In the mitochondrial matrix, Mg is involved in actions of, e.g., α-ketoglutarate dehydrogenase, pyruvate dehydrogenase, glutamate dehydrogenase, etc. And Mg also helps control mitochondrial (and other organelle) volume through control of the K⁺/H⁺ exchanger and its inhibitory effects on the IMM anion channel. One strategy that the cell uses to recognize its deviant behavior and to initiate apoptotic cell death involves IMAC activation and PTP (permeability transition pore) opening.

Mg is also essential for producing (GSH), a major intracellular antioxidant. GSH is probably the most active oxygen species (ROS) scavenger, protecting mitochondrial and other cell components against the reactive oxygen species (ROS) that can oxidize and disable most cell components, including lipids, nucleic acids and polypeptides.

Mg must be understood to be innately involved in most important cellular and organelle metabolic pathways. Any factor impacting Mg activities thus has profound effect on the cell. The sensitivity of Mg binding to H⁺ concentration is one factor behind the emphasis on pH as a harbinger of cell damage.

The organism and its cells have homeostatic systems to control Ca, Mg, and P. Ca and Mg are divalent cations when free in aqueous solution. P, phosphorus, is usually found in the form of a phosphate (PO₄⁻³) or bound or unbound phosphate derivative. For the organism, homeostasis is achieved by the coordinated actions of intestine, which controls absorption from ingested foods; the kidney, which modulates excretion of nitrogen and other metabolites, and controls basal pH; the lung, which balances O₂, CO₂, and circulating pH, short term; and the skeleton, which acts as a bank for deposits and withdrawals. Parathyroid hormone (PTH) controls mineral fluxes across intestine, bone, and kidney in concert with 1,25(OH)₂D3, the active form of vitamin D (aka: calcitriol). Free, ionic, cytosolic Mg (Mg⁺²) is only 5-10% of total cellular Mg. Cytosolic concentration is controlled through uptake of Mg by intracellular organelles. Approximately 60% of cell Mg is located within the mitochondria. Mg is a predominant cofactor required for enzyme systems to carry out PO₄ translocations, transcribe and translate nucleic acid, and ATP associated reactions. Mg has a meager response to Ca homeostatic signals, (they are both divalent cations), but appears to have independent homeostatic controls also. One danger to be considered when manipulating Ca or Mg metabolism is that other divalent cations, most fearfully, lead (Pb) and cadmium (Cd), may respond also.

Phosphate, like Ca, is stored in immense quantity in bone hydroxyapatite crystals in bone. Only about 1/7 of PO₄ is in cells with less than 1/1000 in free circulation. Serum PO₄ is largely determined by the efficiency of reabsorption of filtered PO₄. PO₄ is depleted from serum into cells by endogenous or exogenous insulin. Low PO₄ in serum can reduce the [Ca]x[PO₄] product sufficiently to demineralize bone. And when [PO₄] is elevated crystalline deposits can form at undesirable locations.

Ubiquitination

Ubiquitination has multiple effects on proteins. It may mark the attached protein for degradation via the proteasome. It may assist in transporting a macromolecule to a target location. It may activate, speed u, slow down or inactivate a protein's functions. Ubiquitination is a multi-step process that can culminate in ubiquitin's C-terminal glycine carboxyl group: a) isopeptide bonding to a target's lysine residue(s), b) thioester bonding to a target's cysteine residue(s), c) ester bonding to a target's serine or threonine residue(s), and/or d) peptide bonding to the targets N-terminal amino group. Ubiquitin can also self-ubiquitinate through the terminal carboxyl bonding to another ubiquitin's 7 available lysine residues or to its N-terminal methionine. These bonds are not spontaneous but are catalyzed and controlled by ubiquitin-activating enzymes, at least the first of which comprises a Mg-dependent ATPase that de-energizes the attached ATP molecule to AMP.

The specific lysine residue to which the C-terminus bonds in the polyubiquitination formation directs the fate of the target, e.g., for proteasome degradation or intracellular transport.

Ubiquitination is especially relevant in the context of nuclear proteins. In the nucleus, one of the organelles in eukaryotic cells, histones help organize genetic material. Modification of these nuclear proteins can influence access to the genetic material of the chromosome. Ubiquitination, methylation, acetylation, phosphorylation, ribosylation and most other protein modifications will affect DNA integrity, exposure to damaging molecules, DNA repair, and expression of proximal genes. Carboxy tails of histone proteins H2A and H2B are frequent targets of ubiquitination whereas histones H3 and H4 are targets for methylation, acetylation and phosphorylation. RING domain containing enzymes—RING1A and RING1B work in concert to ubiquitinate histone H2A and repress transcription. 2A-HUB, BRCA1 and BARD1 are examples of other ubiquitinating repressors. Rad6, RNF20/40, UbcH6 and RAD6A/B are ubiquitinating activators of expression. Many deubiquitinating enzymes activate transcription. USP16, USP21, 2A-DUB, BAP1 and USP22 are examples of deubiquitinating transcription activators. Ubiquitinating histones alters the 3-dimensional chromatin structure to expose DNA for transcription. And ubiquitinating a histone subunit can alter histone availability for protein factor binding for initiating or inhibiting transcription.

Ubiquitination is a prominent feature controlling transcription, the birth of proteins as it is also intertwined in protein death through degradation in the proteasome. The proteasome pathway must function properly for successful differentiation and development, including ribosome assembly, cell cycling and mitosis, recycling organelles and parts thereof, control of intracellular signaling, membrane receptor activity, apoptosis and inflammatory responses.

Ubiquitination is notably relevant in the context of NFkB and its regulated pathways. NFkB regulates expression of multiple proteins. For example, when NFkB no longer supports expression of TRAF1 or TRAF2, their anti-apoptotic activity is lost and these cells are lost to apoptosis. On the other hand when NFkB is activated it supports expression of genes that foment the cell's proliferation. Constitutive expression of NFkB is found is some cancers. In other cancers, the cells adapt to maintain production of the transcription factors that support NFkB.

Several ubiquitin-like peptides and systems have developed in parallel. For example, Nedd8 attached to a protein belonging to the Cullin family can interact with ring finger proteins Rbx1/Roc1 or Rbx2/Roc2 to make an E3 ligase complex that targets the substrate for proteasomal degradation.

De-Ubiquitination

About one hundred deubiquitinizing proteins have been identified. These “DUBs” might rescue the detached substrate from proteasomal degradation, but also can alter function or determine location of the rescued protein. Often deubiquitinating enzymes are in the same complexes harboring ubiquitinated proteins. Thus, switching can be rapid and responsive to, for example, binding a single protein or phosphorylating a protein in the complex. Ubiquitinating and deubiquitinating are reversible response elements for modulating aspects of metabolism.

G Protein Family

Another gene/protein family prevalent in metabolism and proliferation is the G protein family. In excess of 1 in 50 human genes encode G protein receptors. When activated (often by a signal molecule binding to a transmembrane receptor) these receptors are switched on when ligand binding displaces GDP from the receptor and renders a GTP binding site available. This GTP is decomposed to phosphorylate a target protein thereby often initiating a cascade of transports and reactions that deliver the ultimate message (such as via a transcription factor) to its ultimate locale of action. The G protein is effectively turned off by its first phosphorylation reaction and can rebind GDP to await the next activation cycle. One such G protein is the Ras family of which several members are ubiquitin modified to alter activity and location within the cell. Ubiquitin and DUBs must maintain a balance to control G protein activity and act through positive and negative feedback arrangements to turn on or off gene expression, protein location, phosphorylation state, etc., appropriate to the signal ligand's binding to the receptor.

A ras family involvement has been proposed at least for neurological pathologies. Ras family member, Rap1, is characteristically localized at the neurite tip where it regulates the tips ability to extend. When a stimulus polarizes the neuron, Rap1 ubiquitination through the E3 ligase, Smurf2 leads to Rap1 degradation and arrest of neurite extension. But in one of the cell's neurites, Rap1 remains activated and can, at that neurite, facilitate microtubule extension and allow this neurite to develop into the neuron's axon. In an analogous fashion, Rap2 is neddylated by the ubiquitin-like protein Nedd8. This decreases Rap2 activity, blunted downstream signaling and leads to dendrite extension.

Reactive Oxygen Species—ROS—and Defenses

During mitochondrial reactions to generate ATP as the energy molecule for cell functions, imperfections in the pathways cause a fraction of electrons to be transferred directly to O₂. This makes a superoxide anion (O₂⁻), an ionic free radical due to the unpaired electron. The O₂⁻ radical; can react to form other ROS and/or reactive nitrogen species (RNS), e.g., peroxinitrite. Since mitochondria are the primary intracellular site of electron transport chain activity and oxygen consumption, the O₂⁻ anion produced makes mitochondria the major site of ROS generation. In accordance with this, it is generally believed that the normal concentrations of superoxide anion (O₂⁻) in the mitochondrial matrix are 5- to 10-times that found in surrounding cytosol. It is not unexpected therefore that ROS damage is strongest in mitochondria. Normal ROS generation is part of metabolism and cells/mitochondria generally manage the ROS well. One major scavenger is the Mg dependent GSH of the mitochondrion and also in the cytosolic space. ROS scavengers and intracellular repair mechanisms prevent the oxidative effects of ROS from inflicting permanent damage to the cell or its organelles. When scavenging and repairs fail, a relevant back-up plan requires the cell to initiate death through apoptosis. Although ROS species can be generated throughout the cell, including cytosol, peroxisomes, plasma membrane, and ER, the mitochondrial ETC is the main cellular generator of ROS under most physiological circumstances. While classic electron transport in mitochondrial matrices involves four-electron reduction of O₂ to water, partial reduction reactions can occur under physiological conditions. The result is release of superoxide anion (O₂⁻), hydrogen peroxide (H₂O₂) and/or extremely reactive hydroxyl radical (·OH). Complex I and complex III are the greatest progenitors of mitochondrial O₂⁻ generation. Nevertheless, significant production of ROS in complex II sometimes occurs. Mitochondria possess a special mechanism of mild uncoupling that prevents a marked increase in transmembrane potential and, hence, O₂⁻ production. Mild uncoupling constitutes a first line of mitochondrial antioxidant defense because it decreases O₂⁻ generation. But for the small amount of O₂⁻ is still formed, a second line of defense is carried out by cytochrome c (cyt c) dissolved in the aqueous solution in the intermembrane space. Cyt c oxidizes O₂⁻ back to O₂. And this reduced cyt c is then available for oxidation by O₂ via Complex IV. This mechanism is the most effective scavenger of O₂⁻, since the O₂⁻ is converted to O₂. Antioxidants, such as ascorbate, UQ, and α-tocopherol, are present for the mitochondrial antioxidative defense system, but none of these convert O₂⁻ to O₂.

Melatonin participates in mitochondrial homeostasis. Since mitochondria produce high amounts of ROS and RNS and accordingly depend on the GSH uptake from the cytoplasm to maintain GSH redox cycling. Simple antioxidant characteristics of melatonin and its marked ability to increase GSH levels provide important defense against ROS and thus maintain mitochondrial function. Melatonin normally increases the activity Complex I and Complex IV of mitochondrial ETC but has no observable effect on Complex II and Complex III. Melatonin may directly transfer an electron to Complex I to support its activities.

The lipophilic nature of melatonin gives melatonin a strong association with membrane lipids. Melatonin acts to stabilize membranes in which it is bound, e.g., IMM. Improved integrity of the IMM helps maintain transmembrane gradients such as the H⁺ gradient the drives ATP regeneration. The reducing ability of melatonin directly scavenges H₂O₂, a common mitochondrial product derived from O₂⁻. Melatonin, by itself, is capable of supporting mitochondrial ATP production by reducing ROS damage and maintaining the mitochondrial structure.

Supporting mitochondrial ATP generation may restore or rebalance selective advantage towards ETC dependent cells and/or may rebalance a cells metabolism back in the direction of ETC ATP production.

The predominant ROS produced by ETC operation is O₂⁻, a free radical with moderate reactivity. This reactivity can cascade down to more reactive or secondary ROS derivatives. For example, O₂⁻ can undergo dismutation to H₂O₂, a mild oxidant but one that can be converted to the highly reactive hydroxyl radical (in the presence of transition metals (Fe²⁺ and Cu⁺) by means of a Fenton reaction. H₂O₂ has a longer half-life and thus can survive to cross membranes. Accordingly, the ROS cascade can act as a signal secretor releasing one or more ROS species as messenger molecules. ROS can foment destructive force on biomembranes through oxidation of lipid and protein components. Compromised biomembrane integrity (increased permeability), reduced enzymatic availability, effects on transport proteins, and damaged (mutated) nucleic acids are most evident when viewed as altered cell response. The oxidative effects can be neutralized by one or more of the cell's antioxidant systems. The proper function of the scavenging and repair contributors sits on delicate balance that determines the fate and impact of ROS in the cell. A balance of the various anti-oxidant species is also important. For example, if O₂⁻ scavenging activity by SOD exceeds the capacity to dispose of the generated H₂O₂ the more reactive products of H₂O₂ can inflict grave damage.

Accordingly, production and removal of ROS must be controlled in order to avoid oxidative stress damage. When the level of ROS exceeds the detoxifying mechanisms, this is called “oxidative stress”. Oxidative stress is characterized by multiple failures in many pathways and organelles within the cell.

Equilibrium balance between production and detoxifying or scavenging ROS is sensitive to any influence or changed condition that forces a metabolic change. When multiple (even apparently minor) external modifications (nutrition, O₂ concentration, hormone, pH, hydration, etc.) challenge the cell, altered mitochondrial activity puts the cell at risk for increased unmanaged ROS and resultant oxidative stress. The hyper level of ROS can damage most biomolecules, especially lipids (major membrane components), proteins (enzymes, transporters and also major membrane components) and nucleic acids (DNA and RNA). Such ROS induced damage alters membrane properties like permeability, fluidity, ion transport, glucose transport, receptor activity, enzyme activity, protein interaction and cross-linking, protein synthesis, phospholipid synthesis, nucleic acid synthesis, cytoskeletal integrity, virtually any cell function involving two or more compartments. ROS effects on nucleic acids can cause DNA damage, prevent DNA repair, interfere with DNA polymerase, interfere with DNA/RNA binding and so forth. ROS stress can affect multiple areas of the cell and when severe oxidative stress ultimately results in cell death. ROS can be generated by several intracellular organelles or sites, including, but not limited to: cytosol, peroxisomes, plasma membrane, and ER. But, mitochondrial ETC is the main cellular source of ROS in most tissues and cell types in normal physiological circumstances. Normally electron transport in mitochondria involves the four-electron reduction of O₂ to water. But incomplete reduction reactions can occur. These low percentage but frequent “mistakes” will lead to release of superoxide anion (O₂⁻) and H₂O₂. Complex I and complex III are usually the major sources of ROS. The primary ROS resulting from ETC activity is O₂⁻. The extra electron means the molecule has an odd number of electrons and therefore has free radical activity that can lead to more reactive or secondary ROS derivatives being produced in a serial chain of free radical induced reactions. For example, O₂⁻ can undergo dismutation to H₂O₂, a mild oxidant that can be converted to the highly reactive hydroxyl radical in the presence of transition metals, iron and copper (Fe² and Cu⁺) under the Fenton reaction. ROS can react with biomembranes, enzymes, proteins, and nucleic acids, whatever they contact. Antioxidant systems, e.g., glutathione, can scavenge or neutralize ROS and progeny. Since some metabolic functions actually require active oxygen to complete reactions, the generation and scavenging systems are ideally kept in a balance where the compromise minimizes oxidative damage, but maintains sufficient availability of these potentially toxic molecules to carry out the reactions only possible through reduction of one or more of these species.

In most tissues and cell types mitochondria are the main O₂⁻ generators. Of the collective sites that generate O₂⁻ in the mitochondrial matrix, only O₂⁻ from complex III is released in significant amounts both into the matrix and into the IMS. This spatial difference (matrix vs. IMS) may determine whether mitochondrial O₂⁻ is released to the cytoplasm because anionic charge on O₂⁻ limits membrane permeation and since ROS is mostly produced in the mitochondrial matrix we would expect that the bulk of antioxidant defenses to neutralize O₂⁻ and other ROS should reside in the matrix.

A first line of defense against O₂⁻ is the presence of a specific member of the family of metalloenzymes called superoxide dismutases (SODs), MnSOD or SOD2, specifically located in the mitochondrial matrix. This catalyzes the dismutation of O₂⁻ anion in to H₂O₂. The dismutation of O₂⁻ can also occur spontaneously, but the spontaneous reaction is 10⁴ times slower at body temperature than the enzymatic dismutation by SOD2. O₂⁻ released into the IMS can be eliminated by a different SOD isoenzyme (Cu—Zn-SOD, or SOD1), which is found in the cytoplasm of eukaryotic cells—or scavenged by the cytochrome c plus cytochrome c oxidase system. α-tocopherol may also be available to scavenge O₂⁻, as suggested by experiments with sub-mitochondrial particles isolated from mice fed with vitamin-E supplemented diet. Although the dismutation of O₂⁻ by SOD2 is a predominant source of H₂O₂, other reactions generate H₂O₂ in mitochondria. For example, the redox activity of p66Shc within mitochondria has been shown to generate H₂O₂ in the absence of O₂⁻ through oxidation of cytochrome c. P66Shc normally resides in the cytosol where it is involved in signaling from tyrosine kinases to Ras. However, in response to stress, p66Shc translocates to mitochondria and contributes to generating H₂O₂.

Due to the lack of unpaired electrons, H₂O₂ is not a free radical, but is still a potent oxidant that can oxidize mitochondrial components (proteins, lipids, DNA). Besides being a potential source of more reactive free radicals via Fenton reaction, physiological generation of H₂O₂ fulfills a messenger role since H₂O₂ can be transported across membranes by aquaporins, a family of proteins that act as peroxiporin. The detoxification against H₂O₂ in mitochondria occurs mainly through the GSH redox system, including the glutathione peroxidases (Gpxs) and GSH reductases, as well as the presence of peroxiredoxins using the reducing equivalents of NADPH. Besides these antioxidant defenses that ensure H₂O₂ elimination, aquaporins have been shown to modulate mitochondrial ROS generation. In this paradigm, aquaporin 8 silencing, which is specifically expressed in IMM, enhances mitochondrial ROS generation and results in mitochondrial depolarization and cell death. In addition to these conventional sites of mitochondrial ROS generation, it has been recently reported that the branched-chain2-oxoaciddehydrogenase (BCKDH) complex in mitochondria can produce O₂⁻ and H₂O₂ at higher rates than complex I from mitochondria.

Lipid Peroxidation

Lipid peroxidation is a series of sequential oxidation reactions whereby a damaged lipid (a free radical) can pass the unpaired electron to another molecule such as another lipid molecule and so on until the chain is stopped. When ROS level exceeds threshold, enhanced lipid peroxidation initiates in both cell and organelle membranes. The damaged lipids lose structure and in turn, detrimentally impact normal cell functioning. Lipid peroxidation amplifies the initiating oxidative stress through continued production of lipid-derived free radicals that themselves will inflict continued damage by continuing the lipid peroxide chain or by reacting with and damaging proteins and/or nucleic acid components of the cell. ROS mediated damage to cell membranes can be monitored to assess levels of oxidative stress. The stress may be induced by extracellular events, for example: a food, a pharmaceutical intervention, or may be induced by metabolic changes within the cell. The lipid peroxidation chain often ends with production of an aldehyde. Accordingly, aldehyde levels are a reliable approximator of levels of oxidative stress and of the extent of damage to the various cell components.

Two common sites of ROS activity on phospholipids are unsaturated (double) bonds between two carbon atoms and the ester linkages between glycerols and the fatty acids. Accordingly, polyunsaturated fatty acids (PUFAs) found in membrane phospholipids are especially sensitive to ROS. A single hydroxyl radical can lead to peroxidation of many polyunsaturated fatty acids because the reactions involved are self-sustaining chains of reactions where oxidizing a double bond forms another free radical that can attack the next proximate double bond.

Lipid peroxidation progression traverses three distinct stages: initiation, progression, and termination. Vitamin E in its various forms, being a lipid soluble vitamin, focuses antioxidant effect on peroxidized lipids. Organic acids, such as palmitic acid can assist in capping or scavenging peroxidized lipids to prevent continued downstream oxidative damage.

Initiation starts with the rate limiting step of forming superoxide anion (O₂⁻), a free radical, sometimes written as O₂·⁻ or simply O₂·. Alternatively, a hydroxyl radical, OH, or more commonly: ·OH, a neutral radical, can initiate peroxidation progression. The radicals can react with methylene groups of PUFA to form conjugated dienes, lipid peroxy radicals and hydroperoxides. The lipid peroxy radicals formed are highly contagious in that they are able to propagate the chain reaction:

PUFA-OO⁻·+PUFA-OOH⁻→PUFA-OOH+PUFA·

The lipid hydroperoxides from (PUFA-OOH) undergo reductive cleavage by a reduced metal ion, such as Fe²⁺:

Fe²⁺ complex+PUFA-OOH−→Fe³⁺ complex+PUFA-O·+OH⁻.

Many reactive species including, but not limited to: lipid alkoxyl radicals, aldehydes (malonyldialdehyde, acrolein and crotonaldehyde), alkanes, lipid epoxides, and alcohols result from decomposition of lipid hydroperoxide. The lipid alkoxy radical produced, (PUFA-O·), supports continuing chain reactions:

PUFA-O·+PUFA-H→PUFA-OH+PUFA·.

Peroxidation of polyunsaturated fatty acids by ROS attack can disrupt the carbon chains and, thereby, increase membrane fluidity, leakage and permeability to neutral and some charged substances.

ROS Damage on Proteins

Reactive oxygen species are continuously produced in metabolism. In living cells, when the formation of intracellular reactive oxygen species exceeds the cells' antioxidant capacity, oxidative stress damages organic cellular macromolecules e.g., proteins, lipids and DNA. DNA is a particularly concern because damage to DNA will also disrupt activity of proteins the DNA encodes. But ROS also will attack proteins directly.

ROS action on proteins can impact proteins in a variety of ways, some are direct and others indirect. Direct modification may modulate a protein's activity through nitrosylation, carbonylation, disulphide bond formation, and glutathionylation. Proteins may be modified indirectly when they conjugate with breakdown products of fatty acid peroxidation. The inactive proteins may serve as sinks for substrate of undamaged enzymes or may interfere with cytoskeletal or transmembrane transport. The damaged proteins place extra burden on the cell's metabolic processes whereby macromolecular components are disassembled and recycled. Ubiquitination pathways are at risk of being overwhelmed.

As a consequence of excessive ROS production, site-specific amino acid modification, fragmentation of the peptide chain, aggregation of cross-linked reaction products, altered electric charge and increased susceptibility of proteins to proteolysis may result. Tissues injured by oxidative stress generally contain increased concentrations of carbonylated proteins which is a widely used protein marker for destruction. The amino acids in a peptide differ in their susceptibility to attack by ROS. Sulfur containing proteins are especially sensitive to damage from ROS. ROS activity can remove a H atom from a cysteine residue and form a thiyl radical capable of forming disulfide bridges between proteins or within the same protein. This may cause protein agglomeration or if within a protein will likely interrupt the 3-dimensional structure and ability of the protein to catalyze or transport. The cross-linked proteins are less available as substrates for degradation and may stress the cell by preventing normal recycling metabolism: by maintaining a store of unavailable material, by diverting normal recycling resources to disassemble the damaged molecules and/or by inhibiting activity of the degradative enzymes. Methionine and tyrosine are also especially susceptible to ROS attack. While it is possible for extramitochondrial reactions to produce O₂⁻, in normal circumstances mitochondria are the major source of O₂⁻. And only the O₂⁻ produced at complex III appears to be released both into the matrix and the IMS; other sources produce more local effect. The mtDNA molecule resides in the matrix and therefore is exposed to the greatest ROS risk. Therefore, it is not surprising that superoxide dismutases (SODs) are highly active here. MnSOD, aka SOD2, is a version specific to the mitochondrial matrix. SOD2 dismutates O₂⁻ to H₂O₂. In the intermembrane space and in the cytosol, a copper-zinc dismutase, SOD1, is active for dismutating O₂⁻ to H₂O₂. H₂O₂ is a non-free radical and uncharged oxidant. As such H₂O₂ will oxidize cell components, e.g., lipids, proteins, nucleic acids (including those forming organelles). As an uncharged molecule H₂O₂ is readily transported through biologic membranes using aquaporins and thus can influence neighboring cells as a messenger molecule. The Fenton reaction can convert H₂O₂ to additional species of damaging free radicals.

Nitric oxide (NO, sometimes written as ·NO to indicate the unpaired electron status) is another potent free radical manufactured in our cells and which diffuses from the cell to modify local circulation. NO relaxes smooth muscle in arterioles to increase local circulation. By measuring NO in breath, saliva, urine or other source, levels of the gas can be monitored to signal compromised metabolisms. NO also acts in an intracellular messenger capacity to switch on and off various metabolic features local to the NO source. In blood most NO becomes protein bound for example to hemoglobin.

Several nitric oxide synthases in different pathways can react NADPH with O₂ and arginine to produce the free radical NO. A diet high in green leafy vegetables stimulates NO production independently through reduction of food nitrates to NO. Peroxynitrite is a potent oxidant that is generated upon the reaction of O₂⁻ with nitric oxide (NO). Its impact on inactivation of mitochondrial proteins depends on the level of generation in mitochondria. While ETC is the source of O₂⁻, peroxynitrite a mitochondrial nitric acid synthase may not be a primary source of mtNO. As a gas, NO freely diffuses across membranes, so peroxynitrite may derive from extramitochondrial NO that diffuses into mitochondria to react with O₂⁻ generated by ETC.

The free radical status of NO makes it available as an antibiotic secreted by several of our immune cells. NO directly attacks pathogens such as bacteria. Intracellular NO is one of our defenses to control intracellular parasites such as malaria. NO has the ability to disaggregate Fe—S clusters and block the associated Fe—S protein's activities. DNA damage is another NO effect, especially in bacteria and organelles without protective proteins and repair mechanisms. Our immune cells also use NO to induce apoptosis in compromised cells, for example cells with modified receptors or secretions or cells infected by virus.

Glutathione—GSH

Glutathione (GSH) is a tripeptide composed of the amino acids: glutamate, cysteine and glycine Glutathione is a reducing agent especially active against hydroxy radicals, peroxynitrites, and hydroperoxides. GSH is involved in amino acid transport across cell membranes through the γ-glutamyl cycle. Its reductive capacity makes it an essential cofactor for many enzymatic reactions including the rearrangement of protein disulfide bonds.

GSH is synthesized in the cytosol of all mammalian cells via a two-step reaction where glutamate-cysteine ligase ligates the two as γ-glutamylcysteine; then glutathione synthetase adds a glycine. Cysteine is often the limiting reactant with activity of glutamate-cysteine ligase, aka: γ-glutamylcysteine synthetase being the rate limiting step. Glutathione is transported into the nucleus where its accumulation into the nucleus is a significant enabler in the cell cycle, and in cell proliferation. Nuclear sequestration of GSH influences cytoplasmic glutathione availability. Here and elsewhere GSH, plays an important role in oxidative signaling. The nuclear pore complex that allows the diffusion of other ions and small molecules presumably allows glutathione to also enter by diffusion. But an ATP-dependent glutathione carrier is capable of facilitating GSH crossing into the nucleus. The antiapoptotic factor Bcl-2 also can form a pore-like structure that may be important in the recruitment of glutathione into the nucleus. Bcl-2 can enhance mitochondrial glutathione uptake in several cell lines, but the role of Bcl-2 functioning directly in glutathione uptake does not appear required in all cells.

The GSH redox system is a major detoxifier of H₂O₂ within the mitochondrial matrix with NADPH in the presence of peroxiredoxins being an additional detoxifying route. Glutathione (GSH), the most prevalent intracellular thiol compound, is a ubiquitous tripeptide in that it is produced by most mammalian cells. GSH is the primary mechanism of antioxidant defense against reactive oxygen species (ROS) and electrophiles in the cells and organelles. GSH (γ-glutamyl-cysteinyl-glycine) is synthesized de novo through two Mg-dependent and ATP-dependent enzymatic reactions. In the first reaction, cysteine and glutamate are bound in a reaction catalyzed by the γ-glutamylcysteine synthase (γ-GCS) to form γ-glutamylcysteine. This reaction is the rate-limiting step in GSH synthesis. Cysteine availability is usually a limiting factor in this reaction. The second GSH synthesis reaction is catalyzed by glutathione synthetase (GS), where γ-glutamylcysteine is covalently bonded to glycine. The antioxidant action of GSH occurs through the redox-active thiol (—SH) of cysteine that is oxidized as GSH reduces target molecules. When acting on ROS or electrophiles, GSH is oxidized to GSSG, which will be reduced to GSH by the GSSG reductase (GR). Thus, the GSH/GSSG ratio reflects the oxidative state of the cell or location of interest.

While GSH is synthesized exclusively in the cytosol from its constituent amino acids, GSH is distributed in different compartments, including mitochondria, where its concentration in the matrix equals that of the cytosol. This feature and its negative charge at physiological pH imply the existence of specific carriers to import GSH from the cytosol to the mitochondrial matrix, where it plays a key role in defense against respiration-induced reactive oxygen species and in the detoxification of lipid hydroperoxides and electrophiles. As mitochondria have a strategic role in the activation and mode of cell death, mitochondrial GSH has been shown to critically regulate the level of sensitization to secondary hits that induce mitochondrial membrane permeabilization and release of proteins confined in the intermembrane space: that once in the cytosol engage the molecular machinery of cell death. The regulation of mitochondrial GSH and its available role in cell death suggests its modulation may effectively treat prevalent human diseases, such as cancer, fatty liver disease, several autoimmune diseases and Alzheimer's disease. GSH (glutathione) readily reverses between GSH (reduced form) and GSSG (oxidized form). GSH reacts with H₂O₂ to produce water H₂O and GSSG. NADPH-dependent GSSG reductase then restores GSH for its next detoxifying reaction. Since an adequate supply of NADPH is essential to regenerate GSH, its availability normally limits the rate of H₂O₂ reduction by the Gpx enzyme in the glutathione redox system. Of the eight isoforms of Gpx that have been identified in humans, Gpx1 is the major isoform localized in various cellular compartments, including the mitochondrial matrix. Gpx1 is interesting in that the selenium metal is required for its activity. Gpx1 has substrate specificity for H₂O₂ serves as the major H₂O₂ reducing enzyme at least in mitochondria. At normal physiologic pH, GSH is anionic, but as pH decreases, increasing percentages of GSH molecules have transient neutral characteristics and have reduced activity. Since the mitochondrion becomes less acidic when its ETC activities are challenged, the actions of GSH can become stronger.

GSH is especially relevant in mitochondria since this location is the source of ROS production. Increasing protective reactivity from the glutathione system can calm damage and may prevent severe mitochondrial membrane disruption. On the other hand, comprising mitochondrial membrane integrity can elicit cytochrome c release which may cascade through apoptosis.

The powerful oxidant, peroxynitrite, results from H₂O₂ reacting with nitric oxide (NO). This is but one of the additional active species that may cascade when H₂O₂ overwhelms detoxifying capacity. In addition to defending against oxidants and ROS, GSH also offers protection against electrophiles through glutathione-S-transferases (GSTs). Electrophiles are generated by metabolic processes from both endogenous compounds and xenobiotics. GSTs are widely distributed throughout the cell, for example, GSTA1 in mitochondria, alpha, mu, pi, and zeta in cytosol, and MGST1 which binds to membranes. Mitochondrial GSTs have both GSH transferase and peroxidase activities and detoxify harmful byproducts by GSH conjugation or by GSH-mediated peroxide reduction. Of the isoforms found in human mitochondria at least hGSTA4-4, hGSTA1, hGSTA2, and hGSTP1 have peroxidase activity.

New Cells

Cell growth and proliferation require immense energy to process nutrients using many intertwining biosynthetic pathways in the cell to produce two daughter cells from the one. When cells switch from a non-dividing to a dividing state they demonstrate increased reliance on glycolysis. This has been termed the Warburg effect. The Warburg effect occurs early in the path to carcinogenesis. This may be considered a predisposition of the cell towards malignancy or it may be considered a trigger that rebalances the cell's metabolism to benefit though survival selection to spur further adaptations leading to cancer. The strong dependence of cancer cells and many precancer cells on the relatively inefficient glycolysis for their energy production appears at odds with the profound needs for ATP mediated reactions to support cell division.

The Proton (H⁺) Gradient

The hydrogen ion, H⁺, the proton, is the smallest positively charged atomic structure. The H⁺ gradient established across the IMM by the ETC reactions therefore has both chemical and electric considerations. The chemical component of the gradient derives from an approximately 10-fold lower concentration of H⁺ in the matrix v. intermembrane space or cytosol. This produces a net chemical driving force favoring H⁺ entry into the matrix to release the potential chemical energy. Releasing this energy by using ion pumps allows conversion of the proton gradient potential chemical energy to ATP potential chemical energy. The separation of the charged H⁺ produces an electric potential across the membrane, with a strong (^{˜}140 mv) matrix negative, electrical potential. Overall the sparse supply of intramatrix H⁺ produces a matrix pH of about 8 whereas the inter membrane space and cytosol are close to a neutral pH 7. Both the H⁺ chemical gradient and the matrix negative electric potential induce H⁺ flow towards the matrix from the cytosol. So both the chemical and electrical driving forces promote H⁺ flow to synthesize ATP.

In mitochondria, electron transport generates a H⁺ gradient into the IMM. The potential energy stored in this gradient is then used to effect ATP synthesis in the matrix. As a comparison, in chloroplasts (the photosynthetic organelle in plants), a proton gradient is generated across the thylakoid membrane and is used to drive ATP synthesis in the stroma. Hydrogen ion, acidification, pH change, whatever we wish to call it appears to be a universal consideration over broad aspects in biology.

Transport of small molecules across the inner membrane is mediated by membrane-spanning transport proteins and driven by the electrochemical gradient energized by the H⁺. One example is seen in ATP which is exported from the mitochondrion to the cytosol using a transporter that exchanges ATP for ADP. The voltage component of the H⁺ generated electrochemical gradient drives this exchange: ATP is more negative (⁻4) than ADP (⁻3); since ATP is more negative, exchange of ATP out for ADP in is strongly favoured by the electrochemical gradient. Similarly, transport of phosphate (as H₂PO₄⁻) and pyruvate is driven by a strong chemical gradient. Phosphate and pyruvate exchange are coupled in exchange for hydroxyl ions (OH⁻). The OH⁻ concentration gradient is reciprocal that of H⁺ so there is about a 10-fold gradient where the much higher matrix concentration of OH⁻ provides strong chemical inducement to expel the OH⁻. The exchange is neutral from an electrical standpoint because the H₂PO₄⁻ anion has the same charge as OH⁻. This electrically neutral exchange coupled to the chemical gradient using a transmembrane protein to facilitate phosphate/pyruvate transport into mitochondria is therefore energetically favored overall. The transport of ATP and ADP across the inner membrane is mediated by an integral membrane protein, the adenine nucleotide translocator, which transports one molecule of ADP into the mitochondrion in exchange for one molecule of ATP transferred from the mitochondrion to the cytosol. Because ATP carries more negative charge than ADP (−4 compared to −3), this exchange is driven by the voltage component of the electrochemical gradient. Since the proton gradient establishes a positive charge on the cytosolic side of the membrane, the export of ATP in exchange for ADP is energetically favorable. The synthesis of ATP within the mitochondrion requires phosphate ions (Pᵢ) as well as ADP, so Pᵢ must also be imported from the cytosol. This is mediated by another membrane transport protein, which imports phosphate (H₂PO₄⁻) and exports hydroxyl ions (OH⁻). This exchange is electrically neutral because both phosphate and hydroxyl ions have a charge of ⁻1. However, the exchange is driven by the proton concentration gradient; the higher pH within mitochondria corresponds to a higher concentration of hydroxyl ions, favoring their translocation to the cytosolic side of the membrane. Energy from the electrochemical gradient is similarly used to drive the transport of other metabolites into mitochondria. For example, import of pyruvate from the cytosol (where it is produced by glycolysis) is mediated by a transporter that exchanges pyruvate for hydroxyl ions. Other intermediates of the citric acid cycle can shuttle between mitochondria and the cytosol by similar exchange mechanisms

Endoplasmic Reticulum

More than half of the membrane component of most cells comprises endoplasmic reticulum (ER). The ER is intricately involved in cell operations as the site for synthesis, folding, processing, and guiding transport of proteins. Ribosomes are bound to ER membrane and interplay between ER and other organelles such as golgi and mitochondria guides metabolic paths in the cell. The ER is also the source of most membrane lipids for the plasma membrane and membranes of other organelles. The largest pool of available calcium inside most cells resides in ER. Cell growth and division require an extremely high volume of directed ER activity.

ER stress is potentially fatal to cells and can be brought about by various insults to the ER, such as the accumulation of misfolded proteins. Cells normally respond to ER stress by activating the unfolded protein response (UPR). Phosphorylation of the eukaryotic initiation factor 2α (eIF2α)) on a single serine is central to one arm of the UPR and it rebalances proteostasis by temporarily attenuating global messenger RNA (mRNA) translation. In cells with unresolved ER stress this resultant oxidative stress contributes to cell death. The protein eIF2α is also central to signaling networks that integrate oxidative stress and nutrient availability with other translation regulators such as mechanistic target of rapamycin complex 1 (mTORC1).

The ER has stress pathways that are activated by decreased pH (increased extracellular H⁺). When a cell switches from ETC to glycolytic ATP production the lactic acid decreases intracellular and extracellular pH. The local acidosis can act through ER stress pathways to initiate apoptosis in cells in the immediate neighbourhood of the switched cell(s). G-protein coupled receptor 4 (GPR4) activates at least three ER stress pathways (PERK, ATF6, and IRE1) that can lead to the cell's apoptosis. Thus, interfering with this GPR4 protein by molecular or small molecule intervention can interfere with glycolytic cell dominance and tilt selective pressures in favour of the unswitched cells. In addition to GPR4, GPR65 and GPR68 exhibit similar pH sensitivity.

ER like other organelles can be involved in apoptosis initiation and progression. Apoptosis is an important protective mechanism of cell suicide that organisms have available as a brake on unneeded or malfunctioning cells. Hyperproliferation is one form of malfunction. Thus, under normal operations cells tending to hyperproliferate will self-induce apoptosis to spare the organism. But occasionally the hyperproliferating cells adaptations include adaptations inhibiting or blocking the apoptotic pathways. One apoptotic protection path is the Bax protein that when synthesized in the ER and transported to mitochondria is an activator of apoptosis in the cell. But to protect from inappropriate Bax expression an inhibitory protein Bax inhibitor 1 (BI1) is yet another brake on apoptosis. BL1 activity increases as pH decreases. The protein is hypothesized to have developed as a response for protecting cells from transient ischemia. Modifying any of these processes can profoundly affect apoptosis.

Vitamin D

Vitamin D is a secosteroid that is made in the human skin by photoactivation from sunlight. Vitamin D's forms D2 and D3 are biologically inert before activation by two successive hydroxylations in the liver and kidney to become the biologically active 1,25-dihydroxyvitamin D (1,25(OH)₂D). 1,25(OH)₂D's primary biologic effect is controlling serum calcium. 1,25(OH)₂D coordinates Ca²⁺ uptake by increasing efficiency of absorption of dietary calcium and/or through recruitment of stem cells in bone matrix to differentiate into osteoclasts that harvest calcium stores from the bone into the circulation. The renal production of 1,25(OH)₂D is sensitive to serum calcium levels and to parathyroid hormone (PTH). Through researcher's interests in a wide variety of inborn and acquired disorders of vitamin D metabolism—that can lead to both hypo- and hyper-calcemic conditions, 1,25(OH)₂D's effects on differentiation and division cellular processes are seen as being closely tied to metabolic change and cellular adaptation. 1,25(OH)₂D thus not only regulates calcium metabolism which has profound effect on cell's and organelle's activation but participates in controlling proliferation and differentiation of normally metabolizing cells and also of cancer cells. Since Ca²⁺ is so involved in multiple pathways in cells, 1,25(OH)₂D involvement in these pathways is important. 1,25(OH)₂D has significant roles in immune system modulation with possible involvement in autoimmune disease when 1,25(OH)₂D balance is deficient, enhancing insulin secretion and response to insulin with relevance to obesity and metabolic diseases like diabetes, and in down-regulating the renin/angiotensin system with effects on delivery of nutrients, removal of wastes and distributing hormones. Active vitamin D compounds are used for the treatment of osteoporosis, renal osteodystrophy, and psoriasis and are being developed to treat some cancers, hypertension, benign prostate hypertrophy, cardiovascular heart disease, and type I diabetes. Vitamin D2, which comes from yeast and plants, and vitamin D3, which is found in oily fish and cod liver oil and is made in the skin, are major sources of vitamin D. The differences between vitamin D2 and vitamin D3 are a double bond between C22 and C23, and a methyl group on C24 for vitamin D2. Vitamin D2 is about 30% as effective as vitamin D3 in maintaining vitamin D status. Once vitamin D2 or vitamin D3 enters the circulation, it is bound to the vitamin D-binding protein and transported to the liver, where one or more cytochrome P450-vitamin D-25-hydroxylase(s) (CYP27A1, CYP3A4, CYP2R1, CYP2J3) introduces a OH on carbon 25 to produce 25-hydroxyvitamin D [25(OH)D]. 25(OH)D is the major circulating form of vitamin D. Because the hepatic vitamin D-25-hydroxylase is not tightly regulated, an increase in the cutaneous production of vitamin D3 or ingestion of vitamin D will result in an increase in circulating levels of 25(OH)D. Therefore, its measurement is used to determine whether a patient is vitamin D deficient, sufficient, or intoxicated. 1,25(OH)₂D is a lipid based steroid hormone and performs similar to estrogen and other steroid hormones in inducing its biological responses. 1,25(OH)₂D binds to the vitamin D receptor (VDR) in the cytoplasm to change conformation of the receptor to expose the activation function 2 domain located in helix 12 of the receptor. This conformational switch and contact with other cytoplasmic proteins and co-activators which mediates the complex' translocation along the microtubule to enter the nucleus through the nuclear pore complex. Then in the nucleus, the VDR-1,25(OH)₂D3 complex binds with the retinoid X receptor (RXR). This heterodimeric complex binds to the vitamin D response element (VDRE) to allow binding of multiple initiation factors including, but not limited to: the P160 co-activator proteins glucocorticoid receptor interacting protein 1 (GRIP-1), steroid receptor coactivator-1 (SRC-1), vitamin D receptor interacting protein DRIP-thyroid receptor associated proteins (TRAP) complex, etc., and a collection of coactivators that ultimately initiate transcription of the vitamin D responsive gene. Most tissues and cells in the body have a VDR (vitamin D receptor), including the brain, prostate, breast, gonads, colon, pancreas, heart, monocytes, and T and B lymphocytes. 1,25(OH)₂D has varied biological activities serious physiologic implications. 1,25(OH)₂D3, inhibits proliferation and induces terminal differentiation of normal cells, e.g., keratinocytes and cancer cells that express VDR (including those of the prostate, colon, breast, lymphoproliferative system, and lung). Antiproliferative and pro-differentiating properties of 1,25(OH)₂D3 and its analogs have proved useful in treating the hyperproliferative skin disorder, psoriasis. In kidney 1,25(OH)₂D acts to downregulate renin production with possible profound systemic effect. β-islet cells express a VDR which when activated by 1,25(OH)₂D3 stimulates insulin production and secretion. Activated T and B lymphocytes, monocytes, and macrophages all respond to 1,25(OH)₂D, resulting in the modulation of their immune functions with effect on disease management, autoimmune disease events and the immune system's policing activity against modified cells, and significantly cancer cells.

Only a few neutral molecules cross the IMM unassisted. These include small, essentially gaseous molecules, e.g., CO₂, O₂, NO and NH₃, and some of the small carboxylic acids which have similar carbon chain structure to membrane lipids. These apparently cross membranes in their neutral forms (e.g., protonated carboxylic acids).

Most molecules have specific transporter proteins to enable shuttling across membranes, especially the inner mitochondrial membrane. Shuttles are a group of cooperating compounds. Enzymes convert target molecules into metabolites that recognized by transmembrane transporters. Often on the other side of the membrane the converted target molecule is back converted to its pre-crossing form. Aspartate is an important cog for transporting electrons in the mitochondrial Electron Transport Chain (ETC).

The electrons of NADH must be transported from the cytoplasmic space into the mitochondria to drive production of ATP. Because the NADH itself cannot cross the mitochondrial membrane, one important shuttle responsibility is the transport of reducing equivalents through to the inner mitochondrial membrane. Two separate shuttles are available to accomplish this: the glycerophosphate shuttle and the Malate-Aspartate shuttle.

Malate dehydrogenase is actually a pair of enzymes, one form in the mitochondrial matrix and a second form in the cytoplasm. In the cytoplasm that enzyme reacts on oxaloacetate and NADH to form malate and NAD⁺. An electron and H are transferred to oxaloacetate producing malate. Then malate keto-glutarate antiporter of the inner membrane exchanges α-ketoglutarate from the matric with the cytosolic malate. Once in the matrix, malate dehydrogenase converts malate to make oxaloacetate and NADH. Another enzyme, aspartate aminotransferase in the matrix converts glutamine to α-ketoglutarate and oxaloacetate to aspartate. Another antiporter, the glutamate-aspartate antiporter, exchanges mitochondrial aspartate with cytosolic glutamate. Then in the cytosol cytosolic aspartate aminotransferase to restore oxaloacetate for the next shuttle round.

The net equation for the malate-aspartate shuttle is simple: cytosolic NADH becomes NAD⁺ and mitochondrial matrix NAD⁺ is reduced to NADH. Matrix NADH then feeds the ETC to produce ATP with production of 3 ATP molecules possible for each shuttling cycle. In contrast an alternate shuttling system, the glycerol phosphate shuttle that reduces FAD⁺ to FADH₂ is less efficient resulting in 1 fewer ATP molecule per cycle. This shuttle is one mechanism used by brown fat for generating heat to maintain body temperature.

The Citrate Pyruvate Shuttle

Malate can also act as a cog in the citrate-pyruvate shuttle system across the mitochondrial membrane. Pyruvate, the dissociative product of pyruvic acid in neutral solution once pumped into the matrix using a proton exchanger can be carboxylated by pyruvate carboxylase with consumption of one ATP. This produces oxaloacetate in the matrix. The oxaloacetate might be converted to aspartate or may be acted on by citrate synthase which consumes Acetyl-CoA to CoA-SH and produces citrate. Citrate can be exchanged with extra-matrical malate. Malate exchange to remove it from the matrix is coupled through a phosphate exchange portal.

Extra-matrical citrate then with the help of ATP citrate lyase uses an ATP and CoA-SH to make acetyl-CoA, oxaloacetate and an ADP. This oxaloacetate is reversibly converted to malate generating an NAD which to complete the citrate pyruvate cycle consumes an NADP as malic enzyme produces NADPH (and CO₂) and pyruvate.

This shuttle consumes one ATP one each side of the IMM and has CO₂, NADPH and pyruvate as product. So overall 2 ATP are used to transport acetyl-CoA out of the mitochondria and to transfer electrons from NADH to NADPH.

IMM proteins include, but are not limited to: ETC proteins and protein complexes: ubiquinone (NADH dehydrogenase), electron-transferring-flavoprotein dehydrogenase, electron-transferring flavoprotein, succinate dehydrogenase, alternative oxidase, cytochrome bc1 complex, cytochrome c, cytochrome c oxidase, F-ATPase; ATP-ADP translocase; ATP-binding cassette transporter; cholesterol side-chain cleavage enzyme; protein tyrosine phosphatase; carnitine O-palmitoyltransferase; carnitine O-acetyltransferase; carnitine O-octanoyltransferase; cytochrome P450; translocase of the inner membrane; glutamate aspartate transporter; pyrimidine metabolism: dihydroorotate dehydrogenase, thymidylate synthase (FAD); HtrA serine peptidase 2; adrenodoxin reductase; Heme biosynthesis: protoporphyrinogen oxidase, ferrochelatase; uncoupling protein; etc.

So, with respect to glucose metabolic pathways, glucose can be transported across the plasma membrane and enter the cell. It is phosphorylated [GP] and downgrades one ATP to ADP to become G6P. Glycine can interconvert with G6P.

G6P can follow the glycolysis route through F6P [PI] or may enter the amino acid synthesis pathway [GS] degrading another ATP and adding nitrogen to form glycine. F6P can downgrade another ATP as it is phosphorylated [PFK] to F1,6P and then [G3 Pa] GA3P.

GA3P can be reduced using NADH to Gr3P or it can be oxidized and phosphorylated [G3PDH] to 1,3BPG, then upgrade an ADP as it forms PEP. PEP upgrades another ADP as it forms pyr, pyruvate.

Pyr can convert to alanine and shuttle ammonias out of the cell, may become lactate or may be transported into a mitochondrion. In the mitochondrion pyr is oxidized by NAD⁺ and produces waste CO₂+ to become Acetyl-CoA and then citrate.

Citrate may return to cytoplasm or may be oxidized by NAD⁺ to α-ketoglutarate. Citrate to α-ketoglutarate to succinyl-CoA to succinate to malate to oxaloacetate to citrate are all reversible reaction and can function in either direction. Cytoplasmic malate can cross into the mitochondria and participate in this cycle.

The cytosolic enzymes discussed here are glycogen phosphorylase and phosphoglucomutase [GP], glycogen synthase [GS], phosphoglucose isomerase [PI], phosphofructokinase [PFK], aldolase and triose phosphate isomerase [G3 Pa], glyceraldehyde 3-phosphate dehydrogenase [G3PDH], phosphoglycerate kinase, pyruvate kinase, lactate dehydrogenase, alanine formation (alanine aminotransferase), lipases, glycerol 3-phosphate dehydrogenase, acyltransferase, acyl-CoA synthetase, ATPase, creatine kinase, adenylate kinase, ATP-citrate lyase, acetyl CoA carboxylase, malate dehydrogenase, and malonyl CoA utilization. When PFK converts fructose-6-phosphate into fructose-1,6-bisphosphate (before conversion into glyceraldehyde-3-phosphate and dihydroxyacetone phosphate) the pathway has a branch wherein the dihydroxyacetone phosphate can be diverted into glycerol-3-phosphate and used to form triglycerides. In a counter pathway, triglycerides can be broken down into fatty acids and glycerol. Glycerol can feed the glycolytic pathway though its conversion to dihydroxyacetone phosphate.

Mitochondrial enzymes include Pyruvate Dehydrogenase, Fatty Acyl-CoA Oxidation: acyl-CoA dehydrogenase, enoyl-CoA hydratase, 3-hydroxyacyl-CoA dehydrogenase, and acyl-CoA acetyltransferase, aconitase+isocitrate dehydrogenase, α-ketoglutarate dehydrogenase, succinyl-CoA synthetase, succinate dehydrogenase, malate dehydrogenase, complex I+III+IV, complex II+III+IV, and F₁F₀-ATPase or complex V.

One stress reliever that can help restore mitochondrial balance is creatine in the form of creatine phosphate. Creatine phosphate acts as a reserve for ATP by serving as an ATP battery, for example when muscles are under extreme demand stress. When mitochondria are incapable of producing the needed ATP, reserve ATP is harvested from the creatine compound. This availability protects mitochondria by reducing stress induced ROS. Carnitine is another mitochondrial stress reducer by facilitating transport of several fuel molecules into mitochondria and at the output end by removing some of the toxic byproducts of ATP production. CoQ10 a participant in the electron transport chain for oxidizing glucose to produce CO₂ and ATP is also an antioxidant protecting the mitochondria from ROS attack. Some mitochondrial dysfunctions are rooted in CoQ10 deficiency, so CoQ10 supplementation is often beneficial and rarely if ever deleterious. Creatine, L-carnitine, and CoQ10 supplements may advantageously be part of a “cocktail” for restoring mitochondrial function closer to the default status and/or for treating mitochondrial disease(s).

Peroxisomes

At least 50 different enzymes participate in peroxisome activity. Some are cell type, cell cycle or cell maturation dependent. Many are inducible—only expressed in response to a signal, each of which involved in a variety of biochemical pathways in different types of cells. Peroxisomes, like mitochondria, produce ROS, especially H₂O₂. Catalase is always present in peroxisomes to reduce the H₂O₂ to water (H₂O). The substrates oxidized in peroxisomes include uric acid, amino acids, and fatty acids. Fatty acid oxidation in peroxisomes makes their energy available for metabolism. Human mitochondria share this fatty acid oxidation ability with peroxisomes.

Fatty acid oxidation produces H₂O₂ from dissolved O₂. Then H₂O₂ is decomposed by the catalase, either by conversion to water or by oxidation of another organic compound (designated AH₂). Peroxisomes also participate in lipid and cholesterol biosynthesis. Peroxisomal proteins are synthesized on free ribosomes and imported into peroxisomes as completed polypeptide chains. New peroxisomes are created by division of enlarged peroxisomes. The ER synthesizes phospholipids for import into peroxisomes, using phospholipid transfer proteins. At least two pathways exist to target proteins into peroxisomes. Ser-Lys-Leu (S-K-L) at the carboxy terminus is the most common targeting signal (peroxisome targeting signal 1, or PTS1). A second targeting signal sequence involves the 9 amino acids of the N-terminus (PTS2). PTS1 and PTS2 are picked up by extraperoxisomal receptor proteins to join a translocation complex that mediates transport across the peroxisome membrane. Cytosolic Hsp70 has been implicated in protein import to peroxisomes, but any role of molecular chaperones within peroxisomes is unclear. In contrast to the requirement that proteins be linearized before crossing mitochondrial membranes, the peroxisome carrier system is able to transport at least some folded proteins. The peroxisome shares some characteristics with mitochondria but is very different in function and activity. Supporting peroxisomes and peroxisomic activities often favors mitochondrial ETC reliance and in general tends to favor apoptotic paths over anti-apoptotic paths.

Survival of the Multicellular Organisms Requires Cell Self Sacrifice—Apoptosis

The genetic code has developed complex features that when optimally functioning requires individual cells or tissues to perform at different levels at different times. For example, in human maturation, cells of “baby” teeth must be removed to allow the “adult” teeth space in the jaw. The process called apoptosis is a process available within the cell to elegantly control death of cells, but not of the organism when a cell is no longer of use or when a cell's functions are not supporting the organism. When a cell recognizes that critical mechanisms including its switching off mechanism have malfunctioned or that its functions are no longer being turned on, for example, by occupation of a hormone receptor on the cell's plasma membrane, the cell has several mechanisms that can be initiated to bring about an orderly deconstruction. As cells age, many will become damaged in ways that are not easily repairable. In these cases, it is a normal life function for these cells to self-initiate apoptosis so that they can be replaced by a progeny from one of the retained stem cells. When the switching off mechanism malfunctions metabolism is detrimentally affected and the malfunctioned cell expresses an abnormal metabolism. Generally, this triggers apoptosis and expediently removes the malfunctioning cell. But in rare, but not rare enough cases, the aberrant metabolism and the hosting cell remain in the organism, consuming its resources without appropriate contribution to the whole organism's well-being. In severe cases, these aberrant cells continue to grow and proliferate, forming physical masses that impact the health of surrounding cells and the organism as a whole. In these cases where the cells are not recognizing their maladaptations, fail to undergo apoptosis and further continue to act in a manner of stem cells and to proliferate in a misguided attempt to restore functions of the malfunctioning cell to the whole organism, a massive tumor can result. Possibly because the organism's system recognizes a missing output and sends hormones or cytokines to encourage the damaged cells to fill the gaps, these cells may proliferate at an accelerated pace. Correcting these rare instances to undo tumorigenic activity and allow healthy cells to continue to support the organism is a goal of the present invention.

As an analogous mode of thought, diabetes can be detected by a sweet tasting urine, an increased water intake, increased urination, a breath with fruity or ketone odor, a measurement of the amount of glucose in the blood, an assay of circulating insulin, an assessment of function or insulin receptor, blindness, poor circulation, etc. On a grand scale before, for example, ketosis could be recognized as a sign of diabetes, death from diabetes or circulation problems had to be recognized. Now we can treat diabetics before serious damage by sensing one of these associated signals. If the cells were behaving like the great majority of cells do and were performing metabolic functions in support of the whole organism, there would be no concern. But when these cells function abnormally, the abnormal functions are rooted in enzymatic (or chemical) reactions that are not in the organism's best interest. These reactions may eventually produce obvious manifestations, but the maladaptations on the individual molecule or nano scale must come first. These abnormal reactions will have several effects. First, they may produce compounds that are not normally made by the cells, for example when an incorrect enzyme is expressed. Second, they may produce excess amounts of a metabolite, for example when an alternative pathway is used or a subsequent reaction is not taking place. Third, they may be consuming resources at a rate faster than healthy and starving proximal cell, or the metabolites released to neighboring cells may cause these cells to alter their metabolisms in response. Fourth, the cells may not metabolize wastes from their own cell family or from the organism, in general, and require other means of disposal, such as sweat, urine, breath; or another detoxification pathway within the organism with its abnormal metabolite(s). These switched metabolism events, especially at early stages would not be apparent to a casual outside observer. But the nano scale events are sensed. If the organism could not sense these events, its health would not be affected on the larger scale. The trick therefore is to scale down the therapeutic process to screen for small early switches using gross but sensitive whole body assessment or on a more local scale perhaps by invoking nano scale sensors, or nano sensors for short. These nano-sensors will sense presence of signs that are not casually observable. For example, minor temperature variations possibly including their minor metabolic effects, nano signatures in for example, blood, urine, sweat or breath. A receptor that no longer binds or a receptor that no longer responds to an extracellular signal may be one type of nano event. A receptor that remains in a permanent activation state, perhaps due to its failure to release its ligand intracellularly or extracellularly may shift the cells metabolism or in a more extreme event, for example when the receptor constitutively activates transcription factors synthesis functions can become almost immeasurable. The extracellular proteome, or in some cases, more general proteomic sampling, e.g., from biopsy, skin abrasions, buccal swipes, mucous sample, hair sample, etc. may herald early metabolic switches.

On a whole body scale: sweat, urine, saliva, electromagnetic field, impedance, blood, tears, breath, odor, etc. are examples of characteristics whose change from an earlier more innate state can reveal pathways most affected by the metabolic switches. Sometimes these changes can be ascribed to a single tissue or organ or to a group of tissues or organs most responsible for the observed alterations. Samples may be compared to an individual's earlier sample(s); samples may be compared to samples from similar genetic background—such as a family or race, gender, local population, common water supply, common phenotype or genotype, time of day, exercise protocol, age, etc. Since each individual has its own gene pool (native nDNA and mtDNA) individual differences in metabolism are to be expected, but since all individuals in a species will share a huge majority of genetic material, with greater similarities found among families, local populations—especially partially isolated populations like island inhabitants or communes, segregated groups by e.g.—class or geographic barrier, patterns within a defined group may be used in an algorithm to suggest most desirable or effective interventions that might be applied to rebalance current metabolism towards its more vigorous native status.

Gender differences include different genetic material on the x and y chromosomes, and results of expressions of these genes such as hormonal influences. In some cultures, genetic differences may also reflect dietary differences, behavioral differences, exposure to chemicals, such as cosmetics, etc. Local populations may share similar genetic characteristics, but will also share exposure to local conditions—such as atmospheric or water pollutants, solar exposure, cosmic rays and other radiation effects, etc. A local water supply will provide its set of ions and other dissolved compounds that will be absorbed, used and eliminated from the body.

Specific phenotypes or genotypes will express activities of the gene(s) of interest, the consequences on metabolism and possibly characteristic changes for that genotype or phenotype expected rate of change, susceptibility to genetic mutation, etc. Time of day can be significant because of: for example, cycling or hormones and activity levels, the dietary status, fatigue, etc. Specific exercise protocols may be used to bring out or emphasize patterns of switched metabolism. Since metabolic switching will increase with time as each biochemical reaction builds on previous metabolic events, age will be an important factor in choosing most desirable or effective intervention methods.

Products of metabolism will be secreted from cells into circulation. Analysis of blood will reveal metabolic patterns that result in these secretions. Urine, i.e., blood filtered through a kidney, will vary depending on the blood that feeds it and therefore can reveal metabolic status. Sweat, saliva and tears will change depending on the blood used to produce them an accordingly can help reveal status of metabolism that fed into the bloodstream. Breath will include volatile compounds from the lungs and airways and thus will contain compounds that may have changed with the switched metabolism. The cellular secretions and the body's excretion and retention protocols will affect conductivity and electromagnetic properties of the whole body or parts thereof. Impedance, resistivity, electromagnetic field or aura, and conductivity are measurements that might be taken.

Odor is meant as an indication or volatile compounds that emanate from the body whether or not the human olfactory system can detect each or a group of them. In some instances, a trained animal may be used to sniff out key metabolic status; or electronic chemical sensors may be used to collect the data. Blood also contains DNA released by cells. Blood DNA is generally bound to cells and plasma protein, but nevertheless is available for analysis. Analysis of blood DNA can be used to provide data indicative of which genes have been active and thereby offer a window into active metabolism.

Data can be collected at multiple levels, for example on a single biopsied cell, an individual, a group present in one location, any select sample group. Data can be collected from the same source over time courses to monitor changes with time and rates of these changes. “Big data” and artificial intelligence may be useful for identifying and validating available and more lucrative rebalancing targets and for evaluating effects of practices used for rebalancing. Algorithms developed using the data may be specific to an individual or to any defined group of individuals. In some circumstances a particular population of cells will present with drastically altered metabolism, such as might be evident in a tumor. One option available for practicing this invention applies nano sensing technology, either non-invasively, for example, by sensing breath, urine, etc., or by using nano probes given a physical presence within an organism or in specific adaptations in a selected location within the organism. The selected location may be in the vicinity of the suspected tumor or might be at another site, perhaps where a metabolite of the abnormal cell would be further metabolized: for example, liver, kidney, or simply in a blood vessel. Sensing of metabolic outputs such as chemical products and heat are two important applications of this nano sensing technology and its application to arresting abnormal metabolism and the cells responsible therefor.

Data can also be collected internally, for example by sectional imaging or by concentrating on a particular tissue or organ. Imaging may use non-invasive techniques which may include supplemented marker compounds to accentuate particular aspects. Internal collection may involve tissue biopsy where one or more tissues samples are removed for analysis. Analytical devices may be inserted into the body. These may be markers that would indicate specific areas (tissues) with high concentrations or a target of that marker or perhaps high activity of an enzyme metabolizing the sensor molecule. Small electronic sensors either wired or wireless may be used to collect data. These sensors may take advantage of nanoscale technology to allow passage through circulation and deposition at a targeted site. The sensors may also be couriers and deliver rebalancing material(s) to specific target sites, for example when metabolic switching is more severe in one body segment or in a specific cell type or cell with high levels of expression of a surface marker. Sensors may be designed to be chemically, electrically, and/or physically sensitive.

One special nano sensor has been carried in our bodies throughout our lives. Our microbiome has adapted to the changes our changed metabolisms have served it. Different species or families of organisms within our microbiomes will have adapted their metabolic reactions just as we will have. The microbiome however will also have faced tremendous changes from its predecessors just a century or two back. Bathing and use of body creams, antiperspirants, etc. have constituted major changes in our dermal biome's characteristic environment. Similar changes have wreaked havoc on conditions our various gut microbiomes will have to adapt to.

Cells of our microbiome are semi-independent organisms associated with diverse regions, organs or tissues of our bodies. By harvesting and analyzing various subgroup in our microbiomes (e.g., collecting: stool, blood, saliva, mucus, sweat, dead dermis, deeper dermis, tissue scrapings, etc.) the adaptations of these microbiota will be a window into the adapted systems the host organism has presented to them. The enzymes and other proteins active in various microbes can help elucidate how the host cells in their source regions have progressively adapted their metabolisms. Assaying proteins or reactions of the microbes' proteins can indicate to some degree the source of the microbe and the environment, including for example, an acidic environment rich in lactate, the microbe has adapted to.

Another assessment of the microbial cells would be to sequence individual or collective microbial genomes. Two tracks of analysis might be selected. One would be to use the microbial genes in their adapted, mutated or gene swapped in state as a window to the adapted host metabolism. A second track would be to analyze the microbes for their contributions to the local environment of the host body portion and where warranted seed the microbiome with microbes that can assist in rebalancing the host organism's metabolism in one or a collection of locales, including microbial intervention that my affect a majority or even almost all cells of the host.

Microbiome cells can be used as sensors to assess near instantaneous metabolic events and status and they may be selected or engineered to help rebalance metabolic paths in the cells which provide the microbe's metabolic turf.

One characteristic of cancer cells and cells in early stages towards cancer pathways is decreased oxidative phosphorylation in the mitochondria. During fetal development, a large proportion of our cells support growth and development by forming additional cells through the process of mitosis. In the adult, most cells have differentiated to take on special tasks such as nerve cells, skin cells, liver cells, etc. Post-differentiation these cells specialize at their differentiated tasks and generally turn off epigenetically mitosis supportive pathways. But in each organ a population of cells called stem cells remains less differentiated and maintains ability to divide. Stem cells are necessary to provide ancient healthy cells as the differentiated cells age and accumulate clutter and internal damage. Usually the stem cell divides in an asymmetric fashion producing one task driven differentiated cell that is incapable of further proliferation and another stem cell. The stem cell is not burdened with metabolic demands to support the organism so does not accumulate ROS induced and other damages resulting therefrom. So, in the body not only cancer cells but other cells are capable of dividing. One commonality observed in all cells preparing to divide is a de-emphasis on oxidative phosphorylation through the electron transport chain and a greater reliance on cytosolic glycolysis. Supporting oxidative phosphorylation by activating and maintaining healthy mitochondria will shift ATP production from the proliferation associated glycolysis weighted balance towards more oxidative phosphorylation and thus make cells less capable of division. Restricting caloric intake can force an organism to be more efficient in energy (ATP) production and thus guide the cell towards increased use of the mitochondria's Electron Transport Chains' oxidative phosphorylation pathways and away from glycolysis in the cytoplasm. Restricting caloric intake is known to decrease cancer incidence. It is hypothesized, but not universally accepted that shifting the metabolic energy balance more towards much more efficient oxidative phosphorylation inhibits inappropriate cell division. Thus, supporting OXPHOS in healthy mitochondria may be useful in weakening effects of aging and in many cases slowing metabolic changes necessary for cancers' progressions.

To support normal mitochondrial metabolism, several compounds distinct may be delivered individually or as cocktails as whole body supplements or possibly targeted to a tissue or organ or to cells with one or more distinguishing characteristics of cancer cells. For example, a stilbene derivative, such as pterostilbene, resveratrol, etc., at a dose of 50-500 mg per day, including, but not limited to: about 50 mg, 75, 100, 125, 150, 175, 200, and 250 mg per day can be delivered as a supplement to boost or support functioning mitochondria and their oxidative phosphorylation processes. Similar dosing, adjusted for bio-availability can be expected for most other compounds. Resveratrol has also been reported to suppress inflammation through lipopolysaccharide induced NFKB-dependent COX-2 activation. Piceatannol, epigallocatechin gallate, epicatechin gallate, curcumin, biochanin, quercetin, kaempferol, morin, phloretin, apigenin and daidzein are examples of compounds that can be similarly used or supplemented in delivered compositions.

Cationic amino acid helices or artificial cationic helices will preferentially bind to the mitochondrial inner membrane due to its extreme membrane potential. This binding can collapse the potential and transform the membrane structure leading to swelling and possible rupture. Mitochondrial swelling itself tends to promote apoptosis to cleaning eliminate the affected cell. Chimerizing these helices to a finder sequence such as an antibody fragment like sequence, a viral receptor sequence, an angioreceptor recognizing sequence or the like that recognizes aberrantly metabolizing cells, cancer cells, or regions harboring cancer cells can direct these cells towards apoptosis.

Coenzyme Q10 (CoQ10) can also be supplemented in an organism's diet. CoQ10 is a participant in the Electron Transport Chain activity and acts to support and stimulate oxidative phosphorylation. Thus, a cell in the process of switching metabolism can be rebalanced towards more normal metabolism. Delivering CoQ10 in conjunction with other compounds may augment or synergize effects or may be used to support particular phases of mitochondrial activity with resulting induction of apoptosis and/or inhibition of cell proliferation/division. Coenzyme A (CoA) is especially important for delivering fatty acids to the mitochondrial outer membrane where carnitine palmitoyltransferase 1 exchanges acetyl CoA for carnitine. The reverse occurs inside the mitochondrial inner membrane under the influence of carnitine palmitoyltransferase 2. CoA is synthesized by mitochondrial outer membranes in response to reduced caloric intake. This appears to be one of the compensating responses linking increased ETC and OXPHOS activity to reduced nutrient availability. Supporting CoA activity and its interface with L-carnitine can help shift metabolic balance from glycolysis towards OXPHOS. Pantothenic acid or pantothenate, the acid counter ion, is found in vitamin supplements containing vitamin B5. Vitamin B5 is a precursor of CoA with pantotheine as one of the intermediate compounds. A dimer of pantotheine, pantothine, is an effective means for delivering pantotheine to the body's cells. CoA is not just required for transporting fatty acids to mitochondria, but it also supplies acetyl groups to other enzymes for inactivating or activating genes. B5 shifts the ATP production away from glycolysis and towards the mitochondrial OXPHOS pathway.

L-carnitine is also a glutathione stimulant capable of increasing ETC activity within mitochondria. In addition, L-carnitine assists transport of fatty acids across mitochondrial membranes by replacing CoA as a fatty acid carrier to transport the molecules to the mitochondrion interior for metabolism. Acetyl-L-carnitine is a preferred compound for oral delivery of L-carnitine as it is more efficiently absorbed in the small intestine.

Supplemented acetyl-L-carnitine has been shown to attenuate mitochondrial fission. This feature may be important since it has been observed that cancer cells' mitochondria have elevated fission with respect to fusion. By favoring OXPHOS over glycolysis, interfering with mitochondrial fission, and stimulating glutathione, metabolic shifts associated with neoplastic activity are reversed. Alpha-lipoic acid (or α-lipoic acid) stimulates burning sugar and fatty acids using oxidative phosphorylation. α-lipoic acid stimulates glutathione activity within cells and has widespread effects within cells including increasing mitochondrial function. This dual boosting effect on mitochondria shifts cells towards simple growth development and maintenance and inhibits proliferative activity.

Selenium is a metallic cofactor important for enzymatic function for such enzymes as the glutathione peroxidases. Selenium inhibits mitochondrial fission and thereby shifts the fusion/fission balance in favor of non-proliferation of the cell. Reduced fission is one factor relating to facilitated apoptosis of the cancer cells and probably many other cells with tendency towards hyperproliferation, so selenium also supports initiation of apoptosis-initiated cell death. Oxidized glutathione promotes the oligomerization of the fusion proteins Mfn1, Mfn2 and Opal to activate fusion further shifting the fission/fusion balance in the direction against that of proliferating cells.

Control of levels of Opal is also a possible strategy to be used individually or in concert with other metabolic or mitochondrial modulating interventions. This inner membrane fusion protein appears necessary to maintain fused mitochondria. When the amount is greatly elevated or depressed transient membrane fusion activities occur, but complete fusions disappear. Mfn2 is induced during myogenesis in muscle cells where significant effort is devoted to mutagenesis. Since the mitochondrion has two membranes, complete fusion requires an initial fusion stage involving the outer membrane. Mfn1 and Mfn2 are anchored on the outer membrane and guide the fusion process there. OPA1 resides in the inner membrane. These fusion proteins bring membranes together by forming interlocking coils and using GTP as an energy source driving combination of the membranes. Since fusion has an anti-fission, anti-oncolytic effect it is interesting to note the correlation of obesity with cancer and the observation that obesity correlates with reduced Mfn2 expression.

Repressing Mfn2 causes morphologic and functional breakdown of the mitochondria network through fission. And significantly, reduced Mfn2 availability inhibited glucose oxidation, reduced mitochondrial membrane potential, total cell respiration, and increased mitochondrial proton leak. Mfn2 expression and maintenance of the fused mitochondria in the network is important to mitochondrial metabolism, including OXPHOS, and a properly functioning cell.

In an opposite activity, Drp1, a protein encoded by nDNA and found in the cytoplasm, when phosphorylated at a particular ser residue (637) combines with Mff and Fis1 to fragment the membrane. Many cancer cells have diminished Opal expression indicating that restoring Opal would be a significant signal for more normal metabolism. Remedying this deficit is one means for maintaining larger fused mitochondria in the mitochondrial network.

Mdivi1 inhibits Drp1 fission initiation by preventing the necessary phosphorylation. Supporting Mdivi1 through increased translation and/or expression is one tool for maintaining fused networks.

The size of the mitochondrial network at any given moment arises from the combination of mitochondrial biogenesis (creation of new mitochondrial material) and mitophagy (mitochondrial autophagy, which degrades mitochondria). These processes can respond to the needs of the cell. The increase in both the mitochondrial protein content and the physical size of the mitochondrial network when yeast cells transition from non-respiratory to respiratory conditions is an example of the upregulation of biogenesis to generate increased mitochondrial content. On the other hand, mitophagy is induced when cells experience a variety of stresses. For example, growing yeast cells in nitrogen-depleted media induces both general autophagy and mitophagy to generate nitrogen for essential cellular processes. Biogenesis and mitophagy have to be regulated to maintain the proper mitochondrial content during normal cell growth.

ATP production by mitochondria requires nicotinamide adenine dinucleotide (NAD). Several studies including, but not limited to: J Biol Chem. 2004; Cell Metab. 2011; Mol Pharmacol. 2011, have demonstrated that NAD levels are limiting. The importance of NAD may be understood from its availability from at least four different synthesis pathways.

Nicotinamide, nicotinamide riboside and nicotinic acid are forms of vitamin B₃ and can be delivered orally. Tryptophan is an amino acid and therefore is provided in a protein rich diet. Supplementation with these facilitators of mitochondrial ETC and transmembrane proton gradient opposes glycolysis and thereby favors non-proliferation attributes of the cell.

Dichloroacetate (DCA), a minor contaminant resulting from chlorination of drinking water, is also a strong potentiator of apoptosis. DCA is known to disrupt mitochondrial membranes allowing protons and cytochrome c escape into the cytoplasm. DCA also inhibits synthesis of pyruvate dehydrogenase, an enzyme essential to the glycolytic pathway which proliferating cells favor for ATP production. The forced shift of glycolytic/OXPHOS balance in the direction of non-proliferation slows production of new cells and also facilitates apoptotic activities. The result of DCA supplementation of cells directed towards apoptosis by other means is a more robust drive to initiate apoptosis in the cell. Omega 3, a common fish oil, can also be used to shift the glycolysis/OXPHOS balance in the direction unfavorable to proliferation.

Flavones or flavonoids, for example, 3,3′,4′,5,7-pentahydroxyflavone-2H₂0, 2-phenyl-4H-1-benzopyran-4one, etc., are purified natural plant products or derivatives of natural plant products. Flavones may be supplemented through a diet emphasizing flavone or flavonoid containing fruits and/or vegetables. They are classified by several nomenclatures or groups including, but not limited to: anthocyanins, procyanidins, flavanones, flavones isoflavones, flavonols, flavon-3-ols, etc. Many flavonoid supplements are available commercially in varying degrees of purity from, for example, simply fresh or dried fruit, plant extracts to purified chemical compounds. These supplements may be anti-apoptotic in the sense they have anti-oxidant characteristics. But, for example, a flavonoid like 3,3′,4′,5,7-pentahydroxyflavone may be incorporated into one or more compositions as part of this invention because of it action to inhibit mitochondrial ATPases and thus favor apoptosis. Flavones are reported to increase uptake of lactate into mitochondria which may exert a small but significant pH buffering capacity. Flavones are associated with an increased production of mitochondrial O₂⁻ anions and concomitant apoptotic cell death. In addition to apoptosis induction flavones are involved with cell cycle arrest, caspase activation and inhibition of tumor cell proliferation. One mechanism of flavone/flavonoid activity is especially relevant with respect to cancer cells. Lactate is a co-end product obtained when glycolysis produces ATP. Cancer cells favor the glycolytic pathway over the more efficient mitochondrial ETC. Flavone facilitated lactate delivery of this lactate, produced by the cancer cell's glycolysis shifted metabolism, increases generation of mitochondrial O₂⁻ radicals which shifts the cell towards an apoptotic event. Supplemented flavone shifts the predominantly glycolytic metabolic pathway of neoplastic cells towards the more ETC based metabolism of normal cells. Flavones also arrest cell proliferation (division/mitosis) by halting progression from G₀ to G phases. 3,3′,4′,5,7-pentahydroxyflavone is also reported to activate deacetylase SIRT1 which also supports apoptotic processes. Flavones have been observed to reduce membrane potential and ion fluxes and permeabilities which may further contribute to their cell death promoting effects. 2′,3,4′,5,7-pentahydroxyflavone is another flavonoid discussed herein as an example. Like other flavonoids it has anti-oxidant effect, but also supports some lipid peroxidation. It also induces apoptosis and interferes with proliferation, but arrests at the G₂/M phase interface. It is reported to have endonuclease activity and to suppress NFκB activation which has both anti-cancer and pro-cancer properties. NFκB is a potent inflammatory cytokine the body elicits against some neoplasms, but its inflammatory results are associated with initiation of some cancers. Proteins or derivatives comprising ankyrin repeats or analogues thereof are useful to block NFκB effect. Such blocking compounds may be delivered to a cell or may be provided to the cell by induced intracellular synthesis.

Thyroid hormone at higher concentrations and pharmaceutical achievable amounts mimics that s that boost can result in decreased mitochondrial membrane potential and through this effect and general metabolic stimulus promote production of apoptosis promoting reactive oxygen species.

Another natural factor that can be beneficially manipulated is the biologic membrane, for example, a class of membrane components called ceramides. Ceramides are an interesting group of compounds found chiefly in biologic membrane bilayer. They are amphiphilic molecules that are integral to the lipid bilayer structure of membranes, but when liberated can act as intercellular and intracellular signal molecules. Ceramides have been recognized as favoring mitochondrial fission. Since fission acts as a brake on apoptosis, inhibiting ceramide fissile activity can potentiate apoptosis by restoring the fusion/fission balance to more normal levels and thereby potentiate apoptosis of ceramide inhibited cells. Fumosins, natural mycotoxins frequently found in grain storage bins, and fumosin analogues are particularly effective in this endeavor. Using natural mycotoxins or synthetic mycotoxin like structures, by favoring fused mitochondria can also remove blockades to apoptosis that might impede anti-cancer therapeutic effects of one or more other constituents in a cocktail provided by this invention.

The mitochondrion has two membranes which maintain pH gradients—the inter-membrane space being relatively acidic to both the mitochondrial matrix (most basic) and the cytosol. Drugs permeable through biologic membranes may distribute based on charge with charges determined by protonation state. Several compounds obtain greatly enhanced activity depending on pH. For example, transition or rare earth elements, with multiple oxidation states display pH sensitivity. Gadolinium is one such element whose toxicity may approach lethal levels as pH decreases but is much less toxic in regions of higher pH. Incorporating one of these ions or one of the several peptides that also increase toxicity at low pH into a particle, e.g., a membrane crossing peptide, a lipoprotein, a liposome, a nanoparticle, can effect entry into targeted cells to produce desired toxic affect. When membrane permeability is increased by activation or opening of the mitochondrial permeability transition pores (MPTP) the pH gradient is destroyed as ions up to about 1.5 kilodalton are free to diffuse through the open pores. Hydrogen ions being especially small (just a single proton, 0.001 kilodaltons) transgress rapidly through the openings and destroy the pH gradients. MPTP activation has several pathways including, but not limited to: accumulated Ca²⁺ in mitochondria, increased Ca⁺⁺ flux, inhibiting Ca²⁺ ATPase, reactive oxygen species, increased ER Ca²⁺, diminished transmembrane potential and pro-oxidants. MPTP activation effects apoptosis by several possible mechanisms. Since the proton gradient provides the energy for ATP production, destruction of the proton gradient by opening MPTPs or by other means results in rapid ATP depletion. The lack of ATP has widespread effects, a major one being that ion pumps on the plasma membrane, the membrane encasing the cytosol, cease functioning. For example, Na⁺/K⁺ ATPase and Ca²⁺ ATPase no longer maintain ion gradients leading to cell membrane depolarization. Ca²⁺ ions rapidly accumulate in the cytoplasm causing cell death through necrosis. Cell death through apoptosis can occur when mitochondrial MPTP permeability allows release of cytochrome c and apoptosis related peptides including caspases and apoptosis inducing factor (AIF) into the cytoplasm. If anti-apoptosis defenses are insufficient to counteract apoptosis inducing events, the cell will die a controlled apoptotic death. Betulinic acid, arsenite, CD437, several amphiphilic cationic α-helical peptides, etoposide, doxorubicin, 1-β-d-arabinofuranosyl-cytosine and ionidamine can use MPTP to shift the cell towards apoptosis. Reactive oxygen species are a class of compounds known to induce apoptosis. Ultraviolet or ionizing radiation, transition metal ions and some xenobiotics are methods that have been used to increase reactive oxygen species and to tilt the balance towards apoptosis.

Cis-1-hydroxy-4-(1-naphthyl)-6-octylpiperidine-2-one by increasing production of damaging active oxygens can contribute to or may induce apoptosis. Shifting metabolism from the ETC oxidation pathway towards glycolysis is one means of reducing ROS production. Conversely, emphasizing the OXPHOS mechanism can reverse this anti-apoptotic tilt. AZT a therapeutic compound used to treat acquired immune deficiency virus infection exhibits cellular toxicity in part through increasing reactive oxygen species production.

The MPTP resides in the IMM and does not directly destroy the outer mitochondrial membrane permeability barrier. But the opening of the pore allows a massive flux of particles into the inter-membrane space. As these particles move, water follows the osmotic gradient causing massive swelling and rupture of the OMM. Apoptosis initiating cytochrome c and other pro-apoptotic proteins are thereby released into the cytoplasmic space where apoptosis can transpire.

Still another path is available for mitochondrial compromise to induce apoptosis. Pro-apoptotic proteins, Bak and Bax, can associate in the outer membrane to provide outer membrane permeabilization. As cells normally function, pro-apoptotic Bak and Bax do not associate to cause cell death. Anti-apoptotic influences must be stronger if a cell is to function. Removing stabilizing anti-Bak/Bax influences would tilt the balance towards apoptosis of the affected cells. Accordingly, one aspect of the invention may include modifying expression of anti-apoptotic proteins including, but not limited to: Bcl-xL and Mcl1, that inhibit Bak/Bax permeabilization of the mitochondrial outer membrane. Methods such as RNAi and gene editing, for example, using a method like CRISPR would be effective. For example, when a virus is used to target cancer cells, the virus can include such expression suppressors.

Tumor Necrosis Factor-α induces apoptosis through support of Bak/Bax linked permeabilization of the mitochondrial outer membrane. However, since Tumor Necrosis Factor-α can activate both pro-apoptotic and anti-apoptotic pathways, it is advised to determine which is the dominant affect in the targeted cell before when this strategy is embraced.

Cell surface receptors associated with initiating apoptosis pathways can also be used to tilt the balance in favor of apoptosis. For example, expression and incorporation of Fas into the plasma membrane can augment apoptosis. For example, genetic engineering to facilitate transcription or translation is an elegant tool to achieve this. Ceramides are believed to stimulate expression of Fas into the cell's plasma membrane. Any compound, for example, daunorubicin and the like, that increase ceramide activity may stimulate apoptosis through this path. Depending on cell type and specific cancer inducing or cancer resultant mutations one or another of these cell deaths pathways may be accelerated at different stages of therapy when multiple cocktails are prepared for sequential therapy. Another factor to consider is other treatments the subject may have received or be receiving. For example, COX2 inhibitors at high doses may promote mitochondrial swelling and compound apoptotic influences, but their possible decoupling effect, at some concentrations, may oppose apoptosis. N-acylethanolamines at high concentrations can reduce mitochondrial membrane potential thereby favoring apoptosis, but at lower concentrations has an effect of closing MPTP with an associated anti-apoptotic tendency. Any one or more of the examples mentioned in this application as well as other associated paths may be targeted as rebalancing tools to redirect opportunistic reactions in cells toward metabolic optimization.

The invention may increase its desired outcomes if multiple modes are practiced sequentially. Neoplastic cells are a class of cells known to decline and to change their metabolic characteristics during the disease process. These cell lines may adapt in response to the body's defenses successfully eliminating some cells. Survivors will have developed characteristics allowing survival in the face of the body's defenses. Similarly, treatment if not 100% successful in eliminating all declining cells will leave survivors with survival characteristics differing from the dead cells. Accordingly, a particularly robust embodiment of the present invention features multiple therapeutic interventions on a schedule that changes as the neoplastic cells are expected to mutate for their survival. Adoptive T cells, T cells cloned with a tumor specific antigen receptor, have been partially effective in fighting cancer. T cells are cultured in the presence of tumor cells and those most reactive to the tumor cell surface proteins are cloned. One or more of these clones was then re-infused into the patient to initiate a T-cell driven immune response. A variant of this method identifies the antigen receptor on the T cell and further identifies the binding portion of the receptor. A stabilized receptor (binding fragment) is engineered for insertion into a targeting moiety. The moiety may be completely synthetic, such as a liposome with receptor embedded in its bilayer or may be a modified biological derivative, such as an enucleated cell transporting antiproliferative therapeutics to cancer cells, a biologic body without a nucleus (e.g., an inside out red cell, modified platelet, etc.), a modified virus, a modified immune cell etc.

Several, but unfortunately not all, cancers feature generously hyper-expressed surface proteins or enzymes whose activity can be readily targeted using binding ligands. Biopsies and screening, e.g., protein chip, cDNA analysis, etc. may be used as tools to identify these features for targeting therapies or sometimes for simply assessing progression of cancer or the treatment. However, it should be considered that not all cells will express the same genes. The age of the cell, interaction with neighbor cells, status of mitochondria, availability of a blood supply, etc. may result in differentiation of surface characteristics. The present invention by continuously altering therapeutic approaches explicitly recognizes this likelihood.

However, one commonality of virtually all cancers is the depressed pH in their vicinity, apparently the result of the glycolytic production of lactic acid. The metabolism common to cancer cells results in extraordinary H⁺ production. PET (positive emission tomography) largely confirms this. Cancer cells thus are an extreme example of the metabolic challenged cells addressed in accordance with this invention.

The present invention, though many of its parts can be considered separate or sub-inventions, in its grandest form takes advantage of early intervention to correct metabolic imbalances. As cells age and accumulate histories, suboptimal circumstances will result in reactions, that though in their circumstance may have been optimal at the time and place are not optimal for the long term. The earlier reactions have set in place a cascade of sequelae that in effect snowball through the system, small at first but growing with time, to unbalance cells' metabolisms. The earlier these events can be rebalanced the less invasive and lower cost in money and effort the sufficient rebalancing intervention will be.

1. A method for modulating metabolism in an organism having at least one cell whose metabolism features imbalanced reliance on ATP production by mitochondria, said method comprising:
assessing metabolic balance in said at least one cell;
selecting one or more cells from those assessed, said one or more cells exhibiting metabolic imbalance or belonging to a population of assessed cells with metabolic imbalance; rebalancing said at least one cell's metabolism to favor oxygenation phosphorylation.

2. The method of claim 1 wherein said imbalance comprises an increased lactate:CO₂ ratio.

3. The method of claim 1 wherein said organism is presenting with symptoms associated with a condition selected from the group consisting of: diabetes, cancer, male infertility, Parkinson's disease, Alzheimer's disease, Huntington's disease and Lou Gehrig's disease.

4. The method of claim 1 wherein said rebalancing comprises: providing a target cell with at least two, three, four, five, six, seven, eight, nine, ten, twelve, fifteen, twenty, twenty-five, or thirty compounds selected from the group consisting of: mitochondrial electron transport chain enhancer, dichloroacetic acid, inhibitor of lactate production, compound modulating amino acid availability, compound modulating glucose availability, palmitic acid, ketogenesis inhibitor, PIP₂ pathway modulator, an Fe—S complex disruptor, dehydroascorbic acid, ascorbic acid mTORC1 modulator, B₁₂, a ubiquitination stimulant, a ubiquitination inhibitor, a deubiquitination stimulant, a deubiquitination inhibitor, oxidoreductase stimulator, glutamate dehydrogenase stimulator, aspartate transaminase inhibitor, caveolin 1 modulator, a flavone, a flavonoid, glucose-6-phosphate dehydrogenase inhibitor, 6-phosphogluconolactonase inhibitor, pyruvate dehydrogenase inhibitor, α-ketoglutarate dehydrogenase inhibitor, vitamin K, lactate dehydrogenase inhibitor, moncarboxylate transport inhibitor, staurosporine, omega 3 fat, 6-phosphogluconate dehydrogenase inhibitor, NFkB inhibitor, melatonin, α-ketoglutarate, dichloroacetate, B3, B5, D2 and analogues thereof, an inhibitor of an iron-sulphur protein, D3 and analogues thereof, leucine, isoleucine, valine, GDP, L-carnitine, acetyl-L-carnitine, vitamin B5, resveratrol, CoQ10, α-lipoic acid, selenium, nicotinamide, nicotinamide riboside, nicotinic acid adenine dinucleotide enhancing supplement, vitamin B3, GTP, alanine, tyrosine and melatonin.

5. The method of claim 4 wherein said metabolism modulation restores at least one feature associated with youth-like metabolism in said organism.

6. The method of claim 4 wherein said metabolism modulation rebalances metabolism towards increasing reliance on said organism's mitochondrial generation of ATP in relation to cytosolic generation of ATP.

7. The method of claim 4 wherein said metabolism modulation reduces lactic acid generation in cells of said organism.

8. The method of claim 7 wherein said reduced lactic acid generation occurs in oxygenated cells.

9. The method of claim 4 wherein said providing to said target cell comprises delivery with multiple copies of at least one surface functional group, said at least one surface functional group acting as a binding ligand for a receptor or transport molecule on said one or more cells exhibiting metabolic imbalance or belonging to a population of assessed cells with metabolic imbalance.

10. The method of claim 4 wherein said providing to said target cell comprises delivery with pH sensitive courier particles.

11. The method of claim 4 wherein said compound modulating glucose availability is selected from the group consisting of: dapagliflozin, empagliflozin, canagliflozin, ipragliflozin (ASP-1941), tofogliflozin, sergliflozin etabonate, remogliflozin etabonate (BHV091009), ertugliflozin (PF-04971729/MK-8835), sotagliflozin, and other compounds of the gliflozin class.

12. The method of claim 4 wherein said compound modulating amino acid availability is selected from the group consisting of: d-amino acids, d-alanine, d-cysteine, d-aspartic acid, d-glutamic acid, d-phenylalanine, d-histidine, d-isoleucine, d-lysine, d-methionine, d-asparagine, d-proline, d-glutamine, d-arginine, d-serine, d-threonine, d-valine, d-tryptophan, d-tyrosine, threo-p-hydroxyaspartate, dihydrokainate, and threo-3-benzyloxyaspartate.

13. The method of claim 4 wherein said compound modulating PIP₂ pathway is selected from the group consisting of: aminosteroid, edelfosine, prozosin, propranolol, o-phenanthroline, adrenergic inhibitors including both a and 3 blockers, trazodone, mirtazapine, ergot alkaloids including metergoline, ketanserin, ritanserin, nefazodone, clozapine, olanzapine, quetiapine, risperidone, asenapine MDL-100,907, cyproheptadine, pizotifen, LY-367,265, 2-alkyl-4-aryl-tetrahydro-pyrimido-azepines, AMDA and derivatives, hydroxyzine, 5-MeO-NBpBr, niaprazine, AC-90179, nelotanserin (APD-125) eplivanserin, pimavanserin (ACP-103), volinanserin, thioperamide, JNJ 7777120, atropine, hyoscyamine, scopolamine, diphenhydramine, dimenhydrinate, dicycloverine, thorazine, tolterodine, oxybutynin, ipratropium, mamba toxin MT7, mamba toxin MT1, mamba toxin MT2, pirenzepine, telenzepine, chlorpromazine, haloperidol, rimonabant, cannabidiol, Δ⁹-tetrahydrocannabivarin, ALW-II-41-27BGJ398, FGF401, SSR128129E, SU 54, afatinib, axitinib, cacozatinib, ceritinib, crizotinib, eriotinib, gefitinib, lapatinib, ponatinib, NVP-BHG712, regrorafenib, sunitinib, vandetanib, and JI-101.

14. The method of claim 4 wherein said compound modulating mTORC1 is selected from the group consisting of: rapamycin, everolimus and temsirolimus.

15. The method of claim 4 wherein said flavone or flavonoid is selected from the group consisting of: 3,3′,4′,5,7-pentahydroxyflavone·2H₂O and 2-phenyl-4H-1-benzopyran-4one.

16. The method of claim 4 comprising delivering to said organism a cocktail comprising compounds selected from four, five, six, seven or more of the following classes of bioactive molecules: an ETC activity enhancer, cationic helix, chimera of cationic helix and a cell plasma receptor ligand, mitochondrial fission inhibitor, pyruvate dehydrogenase inhibitor, apoptosis supporting flavonoid, SIRT1 activity enhancer, NO enhancer, mitochondrial permeability transition pore activation, peroxisome proliferation enhancer, a compound having 1,25(OH)₂D3-like activity, H₂O₂ detoxifier, cyclin A activity enhancer, cyclin D activity enhancer, cyclin E activity enhancer, p14ARF activity enhancer, tyrosine protein kinase inhibitor, protein kinase c inhibitor, cholesterol enricher and mGSH depleter.

17. The method of claim 16 wherein said cocktail comprises a compound selected from the group consisting of: CoQ10, carnitine, acetyl-L-carnitine, pantothenic acid, pantothenate, vitamin B5, pantothine:pantotheine dimer, vitamin B3, dichloroacetic acid, a stilbenoid, staurosporine, cholesterol, N-formylmethionine, and spontaneous producers thereof.

18. The method of claim 16 wherein said cocktail comprises a compound selected from the group consisting of: selenium, GSH, GSSG, α-lipoic acid, dichloroacetate, 3,3′,4′,5,7-pentahydroxyflavone, Cis-1-hydroxy-4-(1-naphthyl)-6-octylpiperidine-2-one, pterostilbene, resveratrol, oxaloacetate, 1,25(OH)₂D3, γ-glutamylcysteine, magnesium, aspartate, and spontaneous producers thereof.

19. The method of claim 16 wherein said cocktail comprises an apoptosis supporting compound selected from the group consisting of: anthocyanins, procyanidins, flavanones, flavones isoflavones, flavonols and flavon-3-ols.

20. The method of claim 4, wherein said selecting uses an algorithm based on data obtained from said assessing.

21. The method of claim 20 wherein said algorithm is developed using computer learning or artificial intelligence.

22. The method of claim 4 wherein said assessing makes use of a process selected from the group consisting of: collecting and analyzing blood DNA, collecting and analyzing z biopsy sample, electromagnetic monitoring, measuring at least one metabolic enzyme activity, collecting and analyzing a saliva sample, collecting and analyzing a sweat sample, collecting and analyzing a tar sample, collecting and analyzing a biopsied sample, monitoring impedance, and imaging the body or a portion thereof.

23. The method of claim 22 wherein said assessing comprises comparing data obtained from progressive time periods.

24. The method of claim 22, wherein said assessing comprises evaluating data from a standard appropriate for comparison to the data from the organism.

25. The method of claim 1, wherein said rebalancing comprises decreasing production of a bioactive substance selected from the group consisting of: 6-P-gluconolactone, acetoacetate, 3-hydroxybutyrate, malonyl-CoA, lactic acid, hexokinase, 5-phosphoribosyl-1-phosphate, 5-phosphoribosylamine, alanine, ribose-5-phosphate, mROS, nucleic acid, peroxynitrite, palmitic acid, myristic acid, octanoic acid, fumarate, glucose-1-phosphate, citrate lyase, GSH, Fe—S cluster, Fe—S protein, α-ketoglutarate, glyceraldehyde-3-phosphate dehydrogenase, ATP synthase, p53, p21 and melatonin.

26. The method of claim 1, wherein said rebalancing comprises decreasing activity of a bioactive component, system, event, process or pathway selected from the group consisting of: hexokinase, dihydrotesterone binding, lipid peroxidation, monocarboxylate transporter, adenylosuccinate lyase, ubiquitination, deubiquitination, adenylosuccinate lyase, amidophosphoribosyl transferase, GAR synthase, GAR transtransformylase, FGAM synthase, AIR synthase, AIR carboxylase, SAICAR synthetase, increasing 6-phosphofructo-2-kinase, AICAR transformylase, IMP cyclohydrolase, IκB kinase β, mROS, pyruvate carboxylation, pyruvate dehydrogenase kinase, protein-serine/threonine kinase, c-jun phosphorylation, malonate conversion to fatty acid, mtFASII, dihydrotestosterone binding, Fe—S cluster binding to citrate, pyridoxal phosphate binding to NFS1, aconitase, lipoyl synthase, isocitrate dehydrogenase, α-ketoglutarate dehydrogenase, succinyl-CoA synthetase, succinic dehydrogenase, fumarase, CAV1, H₂O₂ reduction, lipid peroxide reduction, the biotin pathway, the cobalamin pathway, the folate pathway, the lipoic acid pathway, the niacin pathway, the ubiquitin proteosome pathway for protein degradation, the pyridine synthetic pathway, the ubiquinone pathway, the vitamin D pathway, the vitamin E pathway, the vitamin B6 pathway, the vitamin K pathway, the thiamine pathway, the riboflavin pathway, the retinoid pathway, the pantothenic pathway, an ERK pathway, CoQ10 cycling, NAD cycling, dehydroacscorbic acid cycling, malate dehydrogenase, pyruvate carboxylase, citrate synthase, pyruvate dehydrogenase complex, dihydrolipoyl dehydrogenase, PPase2, GSH peroxidase, GSSG reductase, catalase, SLC1, SLC6, SLC7, SLC36, SLC38, SLC43, PRPP synthase, mitochondrial glutaminase, mitochondrial complex I, mitochondrial complex III, mitochondrial complex II, mitochondrial complex IV, NFS1, glutamate-cysteine ligase, glutathione synthetase, glutaredoxin, mitochondrial heat generation, thioredoxin, glutamate dehydrogenase, sirt4, adenine nucleotide translocase, glyceraldehyde-3-phosphate dehydrogenase, apoptosis, ETC, mGSH, ketogenesis, GSH elimination from a cell, levels of protein lipoylation, establishing transmembrane potential across the IMM, establishing H⁺ gradient across the IMM, maintaining H⁺ gradient across the IMM and maintaining transmembrane potential across the IMM.

27. The method of claim 1, wherein said rebalancing comprises increasing production of a bioactive substance selected from the group consisting of: 6-P-gluconolactone, acetoacetate, β-hydroxybutyrate, malonyl-CoA, lactic acid, hexokinase, 5-phosphoribosyl-1-phosphate, 5-phosphoribosylamine, alanine, ribose-5-phosphate, mROS, nucleic acid, peroxynitrite, palmitic acid, myristic acid, octanoic acid, fumarate, glucose-1-phosphate, citrate lyase, GSH, Fe—S cluster, Fe—S protein, α-ketoglutarate, glyceraldehyde-3-phosphate dehydrogenase, ATP synthase, p53, p21 and melatonin.

28. The method of claim 1, wherein said rebalancing comprises increasing activity of a bioactive component, system, event, process or pathway selected from the group consisting of: hexokinase, dihydrotesterone binding, lipid peroxidation, monocarboxylate transporter, adenylosuccinate lyase, ubiquitination, deubiquitination, adenylosuccinate lyase, amidophosphoribosyl transferase, GAR synthase, GAR transtransformylase, FGAM synthase, AIR synthase, AIR carboxylase, SAICAR synthetase, increasing 6-phosphofructo-2-kinase, AICAR transformylase, IMP cyclohydrolase, IκB kinase β, mROS, pyruvate carboxylation, pyruvate dehydrogenase kinase, protein-serine/threonine kinase, c-jun phosphorylation, malonate conversion to fatty acid, mtFASII, dihydrotestosterone binding, Fe—S cluster binding to citrate, pyridoxal phosphate binding to NFS1, aconitase, lipoyl synthase, isocitrate dehydrogenase, α-ketoglutarate dehydrogenase, succinyl-CoA synthetase, succinic dehydrogenase, fumarase, CAV1, H₂O₂ reduction, lipid peroxide reduction, the biotin pathway, the cobalamin pathway, the folate pathway, the lipoic acid pathway, the niacin pathway, the ubiquitin proteosome pathway for protein degradation, the pyridine synthetic pathway, the ubiquinone pathway, the vitamin D pathway, the vitamin E pathway, the vitamin B6 pathway, the vitamin K pathway, the thiamine pathway, the riboflavin pathway, the retinoid pathway, the pantothenic pathway, an ERK pathway, CoQ10 cycling, NAD cycling, dehydroacscorbic acid cycling, malate dehydrogenase, pyruvate carboxylase, citrate synthase, pyruvate dehydrogenase complex, dihydrolipoyl dehydrogenase, PPase2, GSH peroxidase, GSSG reductase, catalase, SLC1, SLC6, SLC7, SLC36, SLC38, SLC43, PRPP synthase, mitochondrial glutaminase, mitochondrial complex I, mitochondrial complex III, mitochondrial complex II, mitochondrial complex IV, NFS1, glutamate-cysteine ligase, glutathione synthetase, glutaredoxin, mitochondrial heat generation, thioredoxin, glutamate dehydrogenase, sirt4, adenine nucleotide translocase, glyceraldehyde-3-phosphate dehydrogenase, apoptosis, ETC, mGSH, ketogenesis, GSH elimination from a cell, levels of protein lipoylation, establishing transmembrane potential across the IMM, establishing H⁺ gradient across the IMM, maintaining H⁺ gradient across the IMM and maintaining transmembrane potential across the IMM.

29. The method of claim 1, wherein said rebalancing comprises disabling production of a bioactive substance selected from the group consisting of: 6-β-gluconolactone, acetoacetate, β-hydroxybutyrate, malonyl-CoA, lactic acid, hexokinase, 5-phosphoribosyl-1-phosphate, 5-phosphoribosylamine, alanine, ribose-5-phosphate, mROS, nucleic acid, peroxynitrite, palmitic acid, myristic acid, octanoic acid, fumarate, glucose-1-phosphate, citrate lyase, GSH, Fe—S cluster, Fe—S protein, α-ketoglutarate, glyceraldehyde-3-phosphate dehydrogenase, ATP synthase, p53, p21 and melatonin.

30. The method of claim 1, wherein said rebalancing comprises eliminating activity of a bioactive component, system, event, process or pathway selected from the group consisting of: hexokinase, dihydrotesterone binding, lipid peroxidation, monocarboxylate transporter, adenylosuccinate lyase, ubiquitination, deubiquitination, adenylosuccinate lyase, amidophosphoribosyl transferase, GAR synthase, GAR transtransformylase, FGAM synthase, AIR synthase, AIR carboxylase, SAICAR synthetase, increasing 6-phosphofructo-2-kinase, AICAR transformylase, IMP cyclohydrolase, IκB kinase β, mROS, pyruvate carboxylation, pyruvate dehydrogenase kinase, protein-serine/threonine kinase, c-jun phosphorylation, malonate conversion to fatty acid, mtFASII, dihydrotestosterone binding, Fe—S cluster binding to citrate, pyridoxal phosphate binding to NFS1, aconitase, lipoyl synthase, isocitrate dehydrogenase, α-ketoglutarate dehydrogenase, succinyl-CoA synthetase, succinic dehydrogenase, fumarase, CAV1, H₂O₂ reduction, lipid peroxide reduction, the biotin pathway, the cobalamin pathway, the folate pathway, the lipoic acid pathway, the niacin pathway, the ubiquitin proteosome pathway for protein degradation, the pyridine synthetic pathway, the ubiquinone pathway, the vitamin D pathway, the vitamin E pathway, the vitamin B6 pathway, the vitamin K pathway, the thiamine pathway, the riboflavin pathway, the retinoid pathway, the pantothenic pathway, an ERK pathway, CoQ10 cycling, NAD cycling, dehydroacscorbic acid cycling, malate dehydrogenase, pyruvate carboxylase, citrate synthase, pyruvate dehydrogenase complex, dihydrolipoyl dehydrogenase, PPase2, GSH peroxidase, GSSG reductase, catalase, SLC1, SLC6, SLC7, SLC36, SLC38, SLC43, PRPP synthase, mitochondrial glutaminase, mitochondrial complex I, mitochondrial complex III, mitochondrial complex II, mitochondrial complex IV, NFS1, glutamate-cysteine ligase, glutathione synthetase, glutaredoxin, mitochondrial heat generation, thioredoxin, glutamate dehydrogenase, sirt4, adenine nucleotide translocase, glyceraldehyde-3-phosphate dehydrogenase, apoptosis, ETC, mGSH, ketogenesis, GSH elimination from a cell, levels of protein lipoylation, establishing transmembrane potential across the IMM, establishing H⁺ gradient across the IMM, maintaining H⁺ gradient across the IMM and maintaining transmembrane potential across the IMM.
